SEQUENCE LISTING (212 sequences)

SEQ ID NO: 1 - protein, 8 aa, Artificial Sequence
  source: /note=“Description of Artificial Sequence Synthetic peptide”
GFTFSSFG

SEQ ID NO: 2 - protein, 8 aa, Artificial Sequence
  source: /note=“Description of Artificial Sequence Synthetic peptide”
ISGDSNTI

SEQ ID NO: 3 - protein, 6 aa, Artificial Sequence
  source: /note=“Description of Artificial Sequence Synthetic peptide”
ARTLAY

SEQ ID NO: 4 - protein, 6 aa, Artificial Sequence
  source: /note=“Description of Artificial Sequence Synthetic peptide”
EDIYNR

SEQ ID NO: 5 - protein, 3 aa, Artificial Sequence
  source: /note=“Description of Artificial Sequence Synthetic peptide”
GAS

SEQ ID NO: 6 - protein, 9 aa, Artificial Sequence
  source: /note=“Description of Artificial Sequence Synthetic peptide”
LQYWSIPWT

SEQ ID NO: 7 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRDNPKNTLFLQMTSLRSEDTAIYYCARTLAYWGQGTLVTVS

SEQ ID NO: 8 - protein, 108 aa, Artificial Sequence
  source: /note=“Description of Artificial Sequence Synthetic polypeptide”
DIQMTQSSSSFSVSLGDRVTITCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 9 - protein, 441 aa, Artificial Sequence
  source: /note=“Description of Artificial Sequence Synthetic polypeptide”
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQLVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 10 - protein, 40 aa, Artificial Sequence
  source: /note=“Description of Artificial Sequence Synthetic polypeptide”
PPKSGDRSGYSSPGSPGTPGSRSRTPSLPTPPTREPKKVA

SEQ ID NO: 11 - protein, 12 aa, Artificial Sequence
  source: /note=“Description of Artificial Sequence Synthetic peptide”
SRTPSLPTPPTR

SEQ ID NO: 12 - protein, 12 aa, Artificial Sequence
  source: /note=“Description of Artificial Sequence Synthetic peptide”
  MOD_RES (8)..(8): Phosphorylated Thr
SRTPSLPXPPTR

SEQ ID NO: 13 - protein, 38 aa, Artificial Sequence
  source: /note=“Description of Artificial Sequence Synthetic polypeptide”
IATPRGAAPPGQKGQANATRIPAKTPPAPKTPPSSGEP

SEQ ID NO: 14 - protein, 10 aa, Artificial Sequence
  source: /note=“Description of Artificial Sequence Synthetic peptide”
KGQANATRIP

SEQ ID NO: 15 - protein, 8 aa, Artificial Sequence
  source: /note=“Description of Artificial Sequence Synthetic peptide”
GFNIKDYY

SEQ ID NO: 16 - protein, 8 aa, Artificial Sequence
  source: /note=“Description of Artificial Sequence Synthetic peptide”
LDPENDHT

SEQ ID NO: 17 - protein, 8 aa, Artificial Sequence
  source: /note=“Description of Artificial Sequence Synthetic peptide”
SYYKYDDY

SEQ ID NO: 18 - protein, 11 aa, Artificial Sequence
  source: /note=“Description of Artificial Sequence Synthetic peptide”
QSLLDSDGKTY

SEQ ID NO: 19 - protein, 3 aa, Artificial Sequence
  source: /note=“Description of Artificial Sequence Synthetic peptide”
LVS

SEQ ID NO: 20 - protein, 9 aa, Artificial Sequence
  source: /note=“Description of Artificial Sequence Synthetic peptide”
WQGTHFPLT

SEQ ID NO: 21 - protein, 114 aa, Artificial Sequence
  source: /note=“Description of Artificial Sequence Synthetic polypeptide”
EVQLQQSGAELVRPGALVKLSCKASGFNIKDYYMHWVRQRPDQGLEWIGWLDPENDHTIYDPRFQDRANLTADTSSNTAYLQLSSLTSEDTAVYYCSYYKYDDYWGQGTTLTVS

SEQ ID NO: 22 - protein, 113 aa, Artificial Sequence
  source: /note=“Description of Artificial Sequence Synthetic polypeptide”
DVVMTQTPLTLSVTIGQPASISCKSSQSLLDSDGKTYLNWLVQRPGQSPKRLIYLVSKLDSGVPDRFTGSGSGTDFTLKINRVEAEDLGVYYCWQGTHFPLTFGAGTKLELKR

SEQ ID NO: 23 - protein, 8 aa, Artificial Sequence
  source: /note=“Description of Artificial Sequence Synthetic peptide”
GFTLSSFG

SEQ ID NO: 24 - protein, 8 aa, Artificial Sequence
  source: /note=“Description of Artificial Sequence Synthetic peptide”
ISSGSSTI

SEQ ID NO: 25 - protein, 6 aa, Artificial Sequence
  source: /note=“Description of Artificial Sequence Synthetic peptide”
SRRLDY

SEQ ID NO: 26 - protein, 11 aa, Artificial Sequence
  source: /note=“Description of Artificial Sequence Synthetic peptide”
KSLLYKDGKTY

SEQ ID NO: 27 - protein, 3 aa, Artificial Sequence
  source: /note=“Description of Artificial Sequence Synthetic peptide”
LMS

SEQ ID NO: 28 - protein, 9 aa, Artificial Sequence
  source: /note=“Description of Artificial Sequence Synthetic peptide”
QQFVEYPLT

SEQ ID NO: 29 - protein, 112 aa, Artificial Sequence
  source: /note=“Description of Artificial Sequence Synthetic polypeptide”
DVQLVESGGGLVQPGGSRKLSCAASGFTLSSFGMHWVRQAPEKGLEWVAYISSGSSTIYYADTVKGRFTISRDNPKNILFLQMTSLRSEDTAIYYCSRRLDYWGQGTSVTVS

SEQ ID NO: 30 - protein, 113 aa, Artificial Sequence
  source: /note=“Description of Artificial Sequence Synthetic polypeptide”
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQFVEYPLTFGTGTKLELQR

SEQ ID NO: 31 - protein, 12 aa, Artificial Sequence
  source: /note=“Description of Artificial Sequence Synthetic peptide”
  MOD_RES (3)..(3): Phosphorylated Thr
  MOD_RES (8)..(8): Phosphorylated Thr
SRXPSLPXPPTR

SEQ ID NO: 32 - protein, 8 aa, Artificial Sequence
  source: /note=“Description of Artificial Sequence Synthetic peptide”
GFTFSGFG

SEQ ID NO: 33 - protein, 8 aa, Artificial Sequence
  source: /note=“Description of Artificial Sequence Synthetic peptide”
GFTFGSFG

SEQ ID NO: 34 - protein, 8 aa, Artificial Sequence
  source: /note=“Description of Artificial Sequence Synthetic peptide”
VSGDSNTI

SEQ ID NO: 35 - protein, 8 aa, Artificial Sequence
  source: /note=“Description of Artificial Sequence Synthetic peptide”
ISGASSTI

SEQ ID NO: 36 - protein, 8 aa, Artificial Sequence
  source: /note=“Description of Artificial Sequence Synthetic peptide”
ISGDGNTI

SEQ ID NO: 37 - protein, 8 aa, Artificial Sequence
  source: /note=“Description of Artificial Sequence Synthetic peptide”
ISGDSNTV

SEQ ID NO: 38 - protein, 8 aa, Artificial Sequence
  source: /note=“Description of Artificial Sequence Synthetic peptide”
ISGDSSTI

SEQ ID NO: 39 - protein, 6 aa, Artificial Sequence
  source: /note=“Description of Artificial Sequence Synthetic peptide”
ENIYNR

SEQ ID NO: 40 - protein, 3 aa, Artificial Sequence
  source: /note=“Description of Artificial Sequence Synthetic peptide”
GAG

SEQ ID NO: 41 - protein, 9 aa, Artificial Sequence
  source: /note=“Description of Artificial Sequence Synthetic peptide”
LQYWSTPWT

SEQ ID NO: 42 - protein, 9 aa, Artificial Sequence
  source: /note=“Description of Artificial Sequence Synthetic peptide”
LQYRSIPWT

SEQ ID NO: 43 - protein, 112 aa, Artificial Sequence
  source: /note=“Description of Artificial Sequence Synthetic polypeptide”
DAQLVESGGGLVQPGGSRKLSCAASGFTFSSFGMRWVRQAPEKGLEWVAYISGDSNTIYYADTVKGRFTISRDNPKNTLFLQMTSLRSEDTAIYYCARTLAYWGQGTLVTVS

SEQ ID NO: 44 - protein, 108 aa, Artificial Sequence; identical to SEQ ID NO: 8
  source: /note=“Description of Artificial Sequence Synthetic polypeptide”

SEQ ID NO: 45 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVTYISGDSNTIYYADTVKGRFTISRDNPKNTMFLQMTSLRSEDTAIYYCARTLAYWGQGTLVTVS

SEQ ID NO: 46 - protein, 108 aa, Artificial Sequence
  source: /note=“Description of Artificial Sequence Synthetic polypeptide”
DIQMTQSSSSFSVSLGDRATITCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 47 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEMGLEWVAYISGDSNTIYYADTVEGRFTISRDNPKNTLSLQMTSLRSEDTAIYYCARTLAYWGQGTLVTVS

SEQ ID NO: 48 - protein, 108 aa, Artificial Sequence
  source: /note=“Description of Artificial Sequence Synthetic polypeptide”
DIQMTQSSSSFSVSLGDRVTITCKASEDIYNRLAWYQQTPGNAPSLLISGASGLEPGVPSRFSGSGSGRDYTLSITSLQTEDVATYYCLQYWSTPWTFGGGTKLEIKR

SEQ ID NO: 49 - protein, 112 aa, Artificial Sequence
  source: /note=“Description of Artificial Sequence Synthetic polypeptide”
DVQLVESGGGLAQPGGSRKLSCAASGFTFSSFGMHWVRQAPEKGLEWVAYISGDSNTIYYADTEKGRFTISRDNPKNALFLQMTSLRSEDTAIYYCARTLAYWGQGTLVTVS

SEQ ID NO: 50 - protein, 108 aa, Artificial Sequence
  source: /note=“Description of Artificial Sequence Synthetic polypeptide”
DIQMTQSSSSFSVSLGDRVTVTCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 51 - protein, 111 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRDNPKNTLFLQMTSLREDTAIYYCARTLAYWGQGALVTVS

SEQ ID NO: 52 - protein, 108 aa, Artificial Sequence
  source: /note=“Description of Artificial Sequence Synthetic polypeptide”
DIQMTQSSSSFSVSLGDRVTITCKASEDIYNRLAWYQQTPGDAPSLLISGASGLETGVPSRFSGSGSGRDYTLSITSLQTEDVATYYCLQYWSIPWTFGGGTKLEIKR

SEQ ID NO: 53 - protein, 112 aa, Artificial Sequence
  source: /note=“Description of Artificial Sequence Synthetic polypeptide”
DVQLVESGGGLVQPGGSRRLSCAASGFTFSSFGMHWVRQAPEKGLEWVAYVSGDSNTIYYADTVKDRFTISRDNPKNTLFLQMASLRSEDTAIYYCARTLAYWGQGTLVTVS

SEQ ID NO: 54 - protein, 108 aa, Artificial Sequence
  source: /note=“Description of Artificial Sequence Synthetic polypeptide”
DIQMTQSSSSFSVSLGDRVTITCKASEDIYNRLAWYQQTPGNAPSLLISGASGLETGVPSRFSGSGSGRDYTLSITSLQTEDVATYYCLQYWSIPWTFSGGTKLEIKR

SEQ ID NO: 55 - protein, 112 aa, Artificial Sequence
  source: /note=“Description of Artificial Sequence Synthetic polypeptide”
DVQLVESGGGLVQPGGSRKLSCAASGFTFSGFGMHWVRQAPEKGLEWVAYISGDSNTIYYADTVKGRFTISRDNPKNTLSLQMTSLRSEDTAIYYCARTLAYWGQGTLVTVS

SEQ ID NO: 56 - protein, 108 aa, Artificial Sequence; identical to SEQ ID NO: 8
  source: /note=“Description of Artificial Sequence Synthetic polypeptide”

SEQ ID NO: 57 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRLTISRDNPKNTLFLQMTSLRSEDTAIYYCARTLAYWGQGTLVTVS

SEQ ID NO: 58 - protein, 108 aa, Artificial Sequence
  source: /note=“Description of Artificial Sequence Synthetic polypeptide”
DIQMTQSSSSFSVSLGDRVTITCKASEDIYNRLAWYQQIPGNAPSLLISGASGLETGVPSRFSGSGSGRDYTLSITSLQTEDVATYYCLQYWSIPWTFGGGTKLEIKR

SEQ ID NO: 59 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRNNPKNTLFLQMTSLRSEDTAIYYCARTLAYWGQGTLVTVS

SEQ ID NO: 60 - protein, 108 aa, Artificial Sequence
  source: /note=“Description of Artificial Sequence Synthetic polypeptide”
DIQMTQSSSSLSVSLGDRVTITCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 61 - protein, 112 aa, Artificial Sequence
  source: /note=“Description of Artificial Sequence Synthetic polypeptide”
DVQLVESGGGLVQPGGSRKLSCAASGFTFSSFGMHWARQAPEKGLEWVAYISGDSNTIYYADTVKGRFTISRDNPKNTLFLQMTSLRSEDTAIYYCARTLAYWGQGTLVTVS

SEQ ID NO: 62 - protein, 108 aa, Artificial Sequence
  source: /note=“Description of Artificial Sequence Synthetic polypeptide”
DIQMTQSSSSFSVSLGDRVTITCKASEDIYNRLAWYQQTPGSAPSLLISGASGLETGVPSRFSGSGSGRDYTLSITSLQTEDVATYYCLQYWSIPWTFGGGTKLEIKR

SEQ ID NO: 63 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRDNPKNTLLLQMTSLRSEDTAIYYCARTLAYWGQGTLVTVS

SEQ ID NO: 64 - protein, 108 aa, Artificial Sequence
  source: /note=“Description of Artificial Sequence Synthetic polypeptide”
DIQMTQSSSSLSVSLGDRVTITCKASEDIYNRLAWYQQTPGNAPSLLISGASGLETGVPSRFSGSGSGRDYTLSITSLQTEDVATYYCLQYWSIPWTFGGGTKLGIKR

SEQ ID NO: 65 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWIAYISGDSNTIYYADTVKGRFTISRDNPKNTLFLQMTSLRSEDTAIYYCARTLAYWGQGTLVTVS

SEQ ID NO: 66 - protein, 108 aa, Artificial Sequence; identical to SEQ ID NO: 8
  source: /note=“Description of Artificial Sequence Synthetic polypeptide”

SEQ ID NO: 67 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGASSTIYYADTVKGRFTISRDNPKNTLFLQMTSLRSEDTAIYYCARTLAYWGQGTLVTVS

SEQ ID NO: 68 - protein, 108 aa, Artificial Sequence; identical to SEQ ID NO: 8
  source: /note=“Description of Artificial Sequence Synthetic polypeptide”

SEQ ID NO: 69 - protein, 112 aa, Artificial Sequence
  source: /note=“Description of Artificial Sequence Synthetic polypeptide”
DVQLVESGGGLVQPGGSRKLSCAASGFTFSSFGMHWARQAPEKGLEWVAYISGDSNTIYYADTVKGRLTISRDNPKNTLFLQTTSLRSEDTAICYCARTLAYWGQGTLVTVS

SEQ ID NO: 70 - protein, 108 aa, Artificial Sequence
  source: /note=“Description of Artificial Sequence Synthetic polypeptide”
DIQMTQSSSSFSVSLGDRVTITCKTSEDIYNRLAWYQQTPGNAPSLLIPGAGGLETGVPSRFSGSGSGRDYTLSITSLQTEDVATYYCLQYWSIPWTFGGGTKLEIKR

SEQ ID NO: 71 - protein, 112 aa, Artificial Sequence
  source: /note=“Description of Artificial Sequence Synthetic polypeptide”
DVQLVESGGGLVQPGGSRRLSCAASGFTFSSFGMHWVRQAPGKGLEWVAYISGDSNTIYYADTVKGRFTISRDNPKNTLFLQMTSLRSEDTAIYYCARTLAYWGQGTLVTVP

SEQ ID NO: 72 - protein, 108 aa, Artificial Sequence; identical to SEQ ID NO: 8
  source: /note=“Description of Artificial Sequence Synthetic polypeptide”

SEQ ID NO: 73 - protein, 112 aa, Artificial Sequence; identical to SEQ ID NO: 63
  source: /note=“Description of Artificial Sequence Synthetic polypeptide”

SEQ ID NO: 74 - protein, 108 aa, Artificial Sequence
  source: /note=“Description of Artificial Sequence Synthetic polypeptide”
DIRMTQSSSSFSVSLGVRVTITCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 75 - protein, 112 aa, Artificial Sequence
  source: /note=“Description of Artificial Sequence Synthetic polypeptide”
DVQLVESGGGLVQPGGSRRLSCAASGFTFSSFGMHWARQAPEKGLEWVAYISGDGNTIYYADTVKGRFTTSRDNPKNTLFLQMTSLRSEDTAIYYCARTLAYWGQGTLVTVS

SEQ ID NO: 76 - protein, 108 aa, Artificial Sequence
  source: /note=“Description of Artificial Sequence Synthetic polypeptide”
DIQMTQSSSSLSVSLGDRVTITCKASEDIYNRLAWYQQTPGSAPSLLISGASGLETGVPSRFSGSGSGRDYTLSITSLQTEDVATYYCLQYWSIPWTFGGGTKLEIKR

SEQ ID NO: 77 - protein, 112 aa, Artificial Sequence
  source: /note=“Description of Artificial Sequence Synthetic polypeptide”
DVQLVESGGGLVQPGGSRKLSCAASGFTFGSFGMHWVRQAPEKGLEWVAYISGDSNTVYYADTVKGRFTISRDNPKNTLFLQMTSLRSEDTAIYYCARTLAYWGQGTLVTVS

SEQ ID NO: 78 - protein, 108 aa, Artificial Sequence
  source: /note=“Description of Artificial Sequence Synthetic polypeptide”
DIQMTQPSSSFSVSLGDRVTITCKASEDIYNRLAWYQQTPGNAPSLLISGASGLETGVPSRFSGSGSGGDYTLSITSLQTEDVATYYCLQYWSIPWTFGGGTKLEIKR

SEQ ID NO: 79 - protein, 112 aa, Artificial Sequence; identical to SEQ ID NO: 7
  source: /note=“Description of Artificial Sequence Synthetic polypeptide”

SEQ ID NO: 80 - protein, 108 aa, Artificial Sequence
  source: /note=“Description of Artificial Sequence Synthetic polypeptide”
DIQMTQSSSSSSVSLGDRVTITCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 81 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTAKGRFAISRDNPKNTLFLQMTSLRSEDTAIYYCARTLAYWGQGTLVTVS

SEQ ID NO: 82 - protein, 108 aa, Artificial Sequence; identical to SEQ ID NO: 62
  source: /note=“Description of Artificial Sequence Synthetic polypeptide”

SEQ ID NO: 83 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRDNPKSTLFLQMTSLRSEDTAIYYCARTLAYWGQGTLVTVS

SEQ ID NO: 84 - protein, 108 aa, Artificial Sequence
  source: /note=“Description of Artificial Sequence Synthetic polypeptide”
DIQMTQSSSSLSVSLGDRVAITCKASENIYNRLAWYQQTPGNAPSLLISGASGLETGVPSRFSGSGSGRDYTLSITSLQTEDVATYYCLQYWSIPWTFGGGTKLEIKR

SEQ ID NO: 85 - protein, 112 aa, Artificial Sequence
  source: /note=“Description of Artificial Sequence Synthetic polypeptide”
DVQLVESGGGLVQPGGSRKLSCAASGFTFSSFGMHWARQAPEKGLEWVAYISGDSNTIYYADAVKGRFTISRDNPKNTLSLQMTSLRSEDTAIYYCARTLAYWGQGTLVTVS

SEQ ID NO: 86 - protein, 108 aa, Artificial Sequence; identical to SEQ ID NO: 60
  source: /note=“Description of Artificial Sequence Synthetic polypeptide”

SEQ ID NO: 87 - protein, 112 aa, Artificial Sequence
  source: /note=“Description of Artificial Sequence Synthetic polypeptide”
DVQLVESGRGLVQPGGSRKLSCAASGFTFSSFGMHWVRQAPEKGLEWVAYISGDSNTIYYAGTVKGRFTISRDNPKNTLFLQMTSLRSEDTAIYYCARTLAYWGQGTLVTVS

SEQ ID NO: 88 - protein, 108 aa, Artificial Sequence
  source: /note=“Description of Artificial Sequence Synthetic polypeptide”
DIQMTQSSSSSSVPLGDRVTITCKASEDIYNRLAWYQQTPGNAPSLLISGASGLETGVPSRFSGSGSGRDYTLSITSLQTEDVATYYCLQYWSIPWTFGGGTKLEIKR

SEQ ID NO: 89 - protein, 112 aa, Artificial Sequence; identical to SEQ ID NO: 61
  source: /note=“Description of Artificial Sequence Synthetic polypeptide”

SEQ ID NO: 90 - protein, 108 aa, Artificial Sequence
  source: /note=“Description of Artificial Sequence Synthetic polypeptide”
DIQMTQSSSSFSVSLGDRVTITCKASEDIYNRLAWYQQTPGNAPSLLISGASGLETGVPSRFSGSGSGRDYTLSVTSLQTEDVATYYCLQYWSIPWTFGGGTKLEIKR

SEQ ID NO: 91 - protein, 112 aa, Artificial Sequence
  source: /note=“Description of Artificial Sequence Synthetic polypeptide”
GVRLVESGGGLVQPGGSRKLSCAASGFTFSSFGMHWVRQAPEKGLEWVAYISGDSNTIYYADTVKGRFTISRDNPESTLFLQMTSLRSEDTAIYYCARTLAYWGQGTLVTVS

SEQ ID NO: 92 - protein, 108 aa, Artificial Sequence; identical to SEQ ID NO: 8
  source: /note=“Description of Artificial Sequence Synthetic polypeptide”

SEQ ID NO: 93 - protein, 112 aa, Artificial Sequence; identical to SEQ ID NO: 7
  source: /note=“Description of Artificial Sequence Synthetic polypeptide”

SEQ ID NO: 94 - protein, 108 aa, Artificial Sequence; identical to SEQ ID NO: 60
  source: /note=“Description of Artificial Sequence Synthetic polypeptide”

SEQ ID NO: 95 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSSTIYYADTVKGRFTISRDNPKNTLFLQMTSLRPEDTAIYYGARTLAYWGQGTLVTVS

SEQ ID NO: 96 - protein, 108 aa, Artificial Sequence
  source: /note=“Description of Artificial Sequence Synthetic polypeptide”
DIQMTQSSSSLSVSLGDRVTITCEASEDIYNRLAWYQQTPGDAPSLLISGASGLETGVPSRFSGSGSGRDHTLSITSLQTEDVATYYCLQYRSIPWTFGGGTKLEVKR

SEQ ID NO: 97 - protein, 112 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRDNPKNTLHLQMTSLRSEDTAIYYCARTLAYWGQGTPVTVS

SEQ ID NO: 98 - protein, 108 aa, Artificial Sequence; identical to SEQ ID NO: 8
  source: /note=“Description of Artificial Sequence Synthetic polypeptide”

SEQ ID NO: 99 - protein, 112 aa, Artificial Sequence; identical to SEQ ID NO: 29
  source: /note=“Description of Artificial Sequence Synthetic polypeptide”

SEQ ID NO: 100 - protein, 113 aa, Artificial Sequence; identical to SEQ ID NO: 30
  source: /note=“Description of Artificial Sequence Synthetic polypeptide”

SEQ ID NO: 101 - protein, 8 aa, Artificial Sequence
  source: /note=“Description of Artificial Sequence Synthetic peptide”
GFTFSNYA

SEQ ID NO: 102 - protein, 7 aa, Artificial Sequence
  source: /note=“Description of Artificial Sequence Synthetic peptide”
ISSGGTI

SEQ ID NO: 103 - protein, 13 aa, Artificial Sequence
  source: /note=“Description of Artificial Sequence Synthetic peptide”
ARVNYDYGYAMDY

SEQ ID NO: 104 - protein, 11 aa, Artificial Sequence
  source: /note=“Description of Artificial Sequence Synthetic peptide”
QSLENSNGNTY

SEQ ID NO: 105 - protein, 3 aa, Artificial Sequence
  source: /note=“Description of Artificial Sequence Synthetic peptide”
RVS

SEQ ID NO: 106 - protein, 9 aa, Artificial Sequence
  source: /note=“Description of Artificial Sequence Synthetic peptide”
LQVTHVPWT

SEQ ID NO: 107 - protein, 118 aa, Artificial Sequence
  source: /note=“Description of Artificial Sequence Synthetic polypeptide”
EVKLVESGGGLVKPGGSLKLSCAASGFTFSNYAMSWVRQNPEKRLEWVASISSGGTIYSPDSVKGRFTISRDNGRNILYLQMSSLRSEDTAMYSCARVNYDYGYAMDYWGQGTSVTVS

SEQ ID NO: 108 - protein, 113 aa, Artificial Sequence
  source: /note=“Description of Artificial Sequence Synthetic polypeptide”
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPWTFGGGTKLEIKR

SEQ ID NO: 109 - protein, 41 aa, Artificial Sequence
  source: /note=“Description of Artificial Sequence Synthetic polypeptide”
  MOD_RES (34)..(34): Phosphorylated Thr
GQKGQANATRIPAKGGGSGGGSGGGSSRTPSLPXPPTREPK

SEQ ID NO: 110 - DNA, 45 bp, Artificial Sequence
  source: /note=“Description of Artificial Sequence Synthetic primer”
tgtaaaacgacggccagtatgaggtkcyytgytsagytyctgrgg

SEQ ID NO: 111 - DNA, 45 bp, Artificial Sequence
  source: /note=“Description of Artificial Sequence Synthetic primer”
tgtaaaacgacggccagtatgaaatgcagctgggtcatsttcttc

SEQ ID NO: 112 - DNA, 38 bp, Artificial Sequence
  source: /note=“Description of Artificial Sequence Synthetic primer”
caggaaacagctatgaccactggatggtgggaagatgg

SEQ ID NO: 113 - DNA, 39 bp, Artificial Sequence
  source: /note=“Description of Artificial Sequence Synthetic primer”
caggaaacagctatgacccagtggatagacagatggggg

SEQ ID NO: 114 - DNA, 45 bp, Artificial Sequence
  source: /note=“Description of Artificial Sequence Synthetic primer”
tgtaaaacgacggccagtatgaagtttccttctcaacttctgctc

SEQ ID NO: 115 - DNA, 46 bp, Artificial Sequence
  source: /note=“Description of Artificial Sequence Synthetic primer”
tgtaaaacgacggccagtatggactccaggctcaamagttttcctt

SEQ ID NO: 116 - DNA, 39 bp, Artificial Sequence
  source: /note=“Description of Artificial Sequence Synthetic primer”
caggaaacagctatgacccagtggatagaccgatggggc

SEQ ID NO: 117 - protein, 12 aa, Artificial Sequence
  source: /note=“Description of Artificial Sequence Synthetic peptide”
GGGSGGGSGGGS

SEQ ID NO: 118 - protein, 16 aa, Artificial Sequence
  source: /note=“Description of Artificial Sequence Synthetic peptide”
  MOD_RES (4)..(4): Phosphorylated Thr
  MOD_RES (9)..(9): Phosphorylated Thr
CSRXPSLPXPPTREPK

SEQ ID NO: 119 - protein, 15 aa, Artificial Sequence
  source: /note=“Description of Artificial Sequence Synthetic peptide”
  MOD_RES (8)..(8): Phosphorylated Thr
SRTPSLPXPPTREPK

SEQ ID NO: 120 - protein, 12 aa, Artificial Sequence
  source: /note=“Description of Artificial Sequence Synthetic peptide”
KDNIKHVPGGGS

SEQ ID NO: 121 - DNA, 337 bp, Artificial Sequence
  source: /note=“Description of Artificial Sequence Synthetic polynucleotide”
gatgttgtgatgacccagactcctctcacattgtcggtaaccattggacaaccagcctccatctcctgcaagtcgagtcagagcctcttagatagtgatggaaagacatatttgaattggttggtacagaggccaggccagtctccaaagcgcctaatctatctagtgtctaaactggactctggagtccctgacaggttcactggcagtggatcagggacagatttcacactgaaaatcaacagagtggaggctgaggatttgggagtttattattgctggcaaggtacacattttccgctcacgttcggtgctgggaccaagctggagctgaaac

SEQ ID NO: 122 - protein, 112 aa, Artificial Sequence
  source: /note=“Description of Artificial Sequence Synthetic polypeptide”
DVVMTQTPLTLSVTIGQPASISCKSSQSLLDSDGKTYLNWLVQRPGQSPKRLIYLVSKLDSGVPDRFTGSGSGTDFTLKINRVEAEDLGVYYCWQGTHFPLTFGAGTKLELK

SEQ ID NO: 123 - DNA, 346 bp, Artificial Sequence
  source: /note=“Description of Artificial Sequence Synthetic polynucleotide”
gaggttcagctgcagcagtctggggctgagcttgtgaggccaggggccttagtcaagttgtcctgcaaagcttctggcttcaacattaaagactactatatgcactgggtgaggcagaggcctgaccagggcctggaatggattggatggcttgatcctgagaatgatcatactatatatgacccgaggttccaggacagggccaatttaacagcagacacttcctccaacacagcctacctgcagctcagcagcctgacatctgaggacactgccgtctattactgttcctactataagtacgatgactactggggccaaggcaccactctcacagtctcctcag

SEQ ID NO: 124 - protein, 115 aa, Artificial Sequence
  source: /note=“Description of Artificial Sequence Synthetic polypeptide”
EVQLQQSGAELVRPGALVKLSCKASGFNIKDYYMHWVRQRPDQGLEWIGWLDPENDHTIYDPRFQDRANLTADTSSNTAYLQLSSLTSEDTAVYYCSYYKYDDYWGQGTTLTVSS

SEQ ID NO: 125 - DNA, 322 bp, Artificial Sequence
  source: /note=“Description of Artificial Sequence Synthetic polynucleotide”
gacatccagatgacacaatcttcatcctccttttctgtatctctcggagacagagtcaccattacttgcaaggctagtgaggacatatataatcggttagcctggtatcagcagacaccaggaaatgctcctagtctcttaatttctggtgcaagcggtttggaaactggagttccttcaagattcagtggcagtggatctggaagggattacactctcagcattaccagtcttcagactgaagatgttgctacttattactgtctacagtattggagtattccgtggacgttcggtggaggcaccaagctggaaatcaaac

SEQ ID NO: 126 - protein, 107 aa, Artificial Sequence
  source: /note=“Description of Artificial Sequence Synthetic polypeptide”
DIQMTQSSSSFSVSLGDRVTITCKASEDIYNRLAWYQQTPGNAPSLLISGASGLETGVPSRFSGSGSGRDYTLSITSLQTEDVATYYCLQYWSIPWTFGGGTKLEIK

SEQ ID NO: 127 - DNA, 340 bp, Artificial Sequence
  source: /note=“Description of Artificial Sequence Synthetic polynucleotide”
gatgtgcagctggtggagtctgggggaggcttagtgcagcctggagggtcccggaaactctcctgtgcagcctctggattcactttcagtagttttggaatgcactgggttcgtcaggctccagagaaggggctggagtgggtcgcatacattagtggtgacagtaataccatctactatgcagacacagtgaagggccgattcaccatctccagagacaatcccaagaacaccctgttcctgcaaatgaccagtctaaggtctgaggacacggccatttattactgtgcaaggacgctcgcttactggggccaagggactctggtcactgtctctgcag

SEQ ID NO: 128 - protein, 113 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISGDSNTIYYADTVKGRFTISRDNPKNTLFLQMTSLRSEDTAIYYCARTLAYWGQGTLVTVSA

SEQ ID NO: 129 - protein, 108 aa, Artificial Sequence; identical to SEQ ID NO: 8
  source: /note=“Description of Artificial Sequence Synthetic polypeptide”

SEQ ID NO: 130 - protein, 108 aa, Artificial Sequence; identical to SEQ ID NO: 8
  source: /note=“Description of Artificial Sequence Synthetic polypeptide”

SEQ ID NO: 131 - protein, 108 aa, Artificial Sequence; identical to SEQ ID NO: 46
  source: /note=“Description of Artificial Sequence Synthetic polypeptide”

SEQ ID NO: 132 - protein, 108 aa, Artificial Sequence; identical to SEQ ID NO: 48
  source: /note=“Description of Artificial Sequence Synthetic polypeptide”

SEQ ID NO: 133 - protein, 108 aa, Artificial Sequence; identical to SEQ ID NO: 50
  source: /note=“Description of Artificial Sequence Synthetic polypeptide”

SEQ ID NO: 134 - protein, 108 aa, Artificial Sequence; identical to SEQ ID NO: 52
  source: /note=“Description of Artificial Sequence Synthetic polypeptide”

SEQ ID NO: 135 - protein, 108 aa, Artificial Sequence; identical to SEQ ID NO: 54
  source: /note=“Description of Artificial Sequence Synthetic polypeptide”

SEQ ID NO: 136 - protein, 108 aa, Artificial Sequence; identical to SEQ ID NO: 8
  source: /note=“Description of Artificial Sequence Synthetic polypeptide”

SEQ ID NO: 137 - protein, 108 aa, Artificial Sequence; identical to SEQ ID NO: 58
  source: /note=“Description of Artificial Sequence Synthetic polypeptide”

SEQ ID NO: 138 - protein, 108 aa, Artificial Sequence; identical to SEQ ID NO: 60
  source: /note=“Description of Artificial Sequence Synthetic polypeptide”

SEQ ID NO: 139 - protein, 108 aa, Artificial Sequence; identical to SEQ ID NO: 62
  source: /note=“Description of Artificial Sequence Synthetic polypeptide”

SEQ ID NO: 140 - protein, 108 aa, Artificial Sequence; identical to SEQ ID NO: 64
  source: /note=“Description of Artificial Sequence Synthetic polypeptide”

SEQ ID NO: 141 - protein, 108 aa, Artificial Sequence; identical to SEQ ID NO: 8
  source: /note=“Description of Artificial Sequence Synthetic polypeptide”

SEQ ID NO: 142 - protein, 108 aa, Artificial Sequence; identical to SEQ ID NO: 8
  source: /note=“Description of Artificial Sequence Synthetic polypeptide”

SEQ ID NO: 143 - protein, 108 aa, Artificial Sequence; identical to SEQ ID NO: 70
  source: /note=“Description of Artificial Sequence Synthetic polypeptide”

SEQ ID NO: 144 - protein, 108 aa, Artificial Sequence; identical to SEQ ID NO: 8
  source: /note=“Description of Artificial Sequence Synthetic polypeptide”

SEQ ID NO: 145 - protein, 108 aa, Artificial Sequence; identical to SEQ ID NO: 74
  source: /note=“Description of Artificial Sequence Synthetic polypeptide”

SEQ ID NO: 146 - protein, 108 aa, Artificial Sequence; identical to SEQ ID NO: 76
  source: /note=“Description of Artificial Sequence Synthetic polypeptide”

SEQ ID NO: 147 - protein, 108 aa, Artificial Sequence; identical to SEQ ID NO: 78
  source: /note=“Description of Artificial Sequence Synthetic polypeptide”

SEQ ID NO: 148 - protein, 108 aa, Artificial Sequence; identical to SEQ ID NO: 80
  source: /note=“Description of Artificial Sequence Synthetic polypeptide”

SEQ ID NO: 149 - protein, 108 aa, Artificial Sequence; identical to SEQ ID NO: 62
  source: /note=“Description of Artificial Sequence Synthetic polypeptide”

SEQ ID NO: 150 - protein, 108 aa, Artificial Sequence; identical to SEQ ID NO: 84
  source: /note=“Description of Artificial Sequence Synthetic polypeptide”

SEQ ID NO: 151 - protein, 108 aa, Artificial Sequence; identical to SEQ ID NO: 60
  source: /note=“Description of Artificial Sequence Synthetic polypeptide”

SEQ ID NO: 152 - protein, 108 aa, Artificial Sequence; identical to SEQ ID NO: 88
  source: /note=“Description of Artificial Sequence Synthetic polypeptide”

SEQ ID NO: 153 - protein, 108 aa, Artificial Sequence; identical to SEQ ID NO: 90
  source: /note=“Description of Artificial Sequence Synthetic polypeptide”

SEQ ID NO: 154 - protein, 108 aa, Artificial Sequence; identical to SEQ ID NO: 8
  source: /note=“Description of Artificial Sequence Synthetic polypeptide”

SEQ ID NO: 155 - protein, 108 aa, Artificial Sequence; identical to SEQ ID NO: 60
  source: /note=“Description of Artificial Sequence Synthetic polypeptide”

SEQ ID NO: 156 - protein, 108 aa, Artificial Sequence; identical to SEQ ID NO: 96
  source: /note=“Description of Artificial Sequence Synthetic polypeptide”

SEQ ID NO: 157 - protein, 108 aa, Artificial Sequence; identical to SEQ ID NO: 8
  source: /note=“Description of Artificial Sequence Synthetic polypeptide”

SEQ ID NO: 158 - protein, 112 aa, Artificial Sequence; identical to SEQ ID NO: 7
  source: /note=“Description of Artificial Sequence Synthetic polypeptide”

SEQ ID NO: 159 - protein, 112 aa, Artificial Sequence; identical to SEQ ID NO: 43
  source: /note=“Description of Artificial Sequence Synthetic polypeptide”

SEQ ID NO: 160 - protein, 112 aa, Artificial Sequence; identical to SEQ ID NO: 45
  source: /note=“Description of Artificial Sequence Synthetic polypeptide”

SEQ ID NO: 161 - protein, 112 aa, Artificial Sequence; identical to SEQ ID NO: 47
  source: /note=“Description of Artificial Sequence Synthetic polypeptide”

SEQ ID NO: 162 - protein, 112 aa, Artificial Sequence; identical to SEQ ID NO: 49
  source: /note=“Description of Artificial Sequence Synthetic polypeptide”

SEQ ID NO: 163 - protein, 111 aa, Artificial Sequence; identical to SEQ ID NO: 51
  source: /note=“Description of Artificial Sequence Synthetic polypeptide”

SEQ ID NO: 164 - protein, 112 aa, Artificial Sequence; identical to SEQ ID NO: 53
  source: /note=“Description of Artificial Sequence Synthetic polypeptide”

SEQ ID NO: 165 - protein, 112 aa, Artificial Sequence; identical to SEQ ID NO: 55
  source: /note=“Description of Artificial Sequence Synthetic polypeptide”

SEQ ID NO: 166 - protein, 112 aa, Artificial Sequence; identical to SEQ ID NO: 57
  source: /note=“Description of Artificial Sequence Synthetic polypeptide”

SEQ ID NO: 167 - protein, 112 aa, Artificial Sequence; identical to SEQ ID NO: 59
  source: /note=“Description of Artificial Sequence Synthetic polypeptide”

SEQ ID NO: 168 - protein, 112 aa, Artificial Sequence; identical to SEQ ID NO: 61
  source: /note=“Description of Artificial Sequence Synthetic polypeptide”

SEQ ID NO: 169 - protein, 112 aa, Artificial Sequence; identical to SEQ ID NO: 63
  source: /note=“Description of Artificial Sequence Synthetic polypeptide”

SEQ ID NO: 170 - protein, 112 aa, Artificial Sequence; identical to SEQ ID NO: 65
  source: /note=“Description of Artificial Sequence Synthetic polypeptide”

SEQ ID NO: 171 - protein, 112 aa, Artificial Sequence; identical to SEQ ID NO: 67
  source: /note=“Description of Artificial Sequence Synthetic polypeptide”

SEQ ID NO: 172 - protein, 112 aa, Artificial Sequence; identical to SEQ ID NO: 69
  source: /note=“Description of Artificial Sequence Synthetic polypeptide”

SEQ ID NO: 173 - protein, 112 aa, Artificial Sequence; identical to SEQ ID NO: 71
  source: /note=“Description of Artificial Sequence Synthetic polypeptide”

SEQ ID NO: 174 - protein, 112 aa, Artificial Sequence; identical to SEQ ID NO: 63
  source: /note=“Description of Artificial Sequence Synthetic polypeptide”

SEQ ID NO: 175 - protein, 112 aa, Artificial Sequence; identical to SEQ ID NO: 75
  source: /note=“Description of Artificial Sequence Synthetic polypeptide”

SEQ ID NO: 176 - protein, 112 aa, Artificial Sequence; identical to SEQ ID NO: 77
  source: /note=“Description of Artificial Sequence Synthetic polypeptide”

SEQ ID NO: 177 - protein, 112 aa, Artificial Sequence; identical to SEQ ID NO: 7
  source: /note=“Description of Artificial Sequence Synthetic polypeptide”

SEQ ID NO: 178 - protein, 112 aa, Artificial Sequence; identical to SEQ ID NO: 81
  source: /note=“Description of Artificial Sequence Synthetic polypeptide”

SEQ ID NO: 179 - protein, 112 aa, Artificial Sequence; identical to SEQ ID NO: 83
  source: /note=“Description of Artificial Sequence Synthetic polypeptide”

SEQ ID NO: 180 - protein, 112 aa, Artificial Sequence; identical to SEQ ID NO: 85
  source: /note=“Description of Artificial Sequence Synthetic polypeptide”

SEQ ID NO: 181 - protein, 112 aa, Artificial Sequence; identical to SEQ ID NO: 87
  source: /note=“Description of Artificial Sequence Synthetic polypeptide”

SEQ ID NO: 182 - protein, 112 aa, Artificial Sequence; identical to SEQ ID NO: 61
  source: /note=“Description of Artificial Sequence Synthetic polypeptide”

SEQ ID NO: 183 - protein, 112 aa, Artificial Sequence; identical to SEQ ID NO: 91
  source: /note=“Description of Artificial Sequence Synthetic polypeptide”

SEQ ID NO: 184 - protein, 112 aa, Artificial Sequence; identical to SEQ ID NO: 7
  source: /note=“Description of Artificial Sequence Synthetic polypeptide”

SEQ ID NO: 185 - protein, 112 aa, Artificial Sequence; identical to SEQ ID NO: 95
  source: /note=“Description of Artificial Sequence Synthetic polypeptide”

SEQ ID NO: 186 - protein, 112 aa, Artificial Sequence; identical to SEQ ID NO: 97
  source: /note=“Description of Artificial Sequence Synthetic polypeptide”

SEQ ID NO: 187 - protein, 108 aa, Artificial Sequence; identical to SEQ ID NO: 8
  source: /note=“Description of Artificial Sequence Synthetic polypeptide”

SEQ ID NO: 188 - protein, 108 aa, Artificial Sequence; identical to SEQ ID NO: 58
  source: /note=“Description of Artificial Sequence Synthetic polypeptide”

SEQ ID NO: 189 - protein, 108 aa, Artificial Sequence; identical to SEQ ID NO: 8
  source: /note=“Description of Artificial Sequence Synthetic polypeptide”

SEQ ID NO: 190 - protein, 108 aa, Artificial Sequence; identical to SEQ ID NO: 78
  source: /note=“Description of Artificial Sequence Synthetic polypeptide”

SEQ ID NO: 191 - protein, 108 aa, Artificial Sequence; identical to SEQ ID NO: 80
  source: /note=“Description of Artificial Sequence Synthetic polypeptide”

SEQ ID NO: 192 - protein, 108 aa, Artificial Sequence; identical to SEQ ID NO: 84
  source: /note=“Description of Artificial Sequence Synthetic polypeptide”

SEQ ID NO: 193 - protein, 108 aa, Artificial Sequence; identical to SEQ ID NO: 60
  source: /note=“Description of Artificial Sequence Synthetic polypeptide”

SEQ ID NO: 194 - protein, 108 aa, Artificial Sequence; identical to SEQ ID NO: 8
  source: /note=“Description of Artificial Sequence Synthetic polypeptide”

SEQ ID NO: 195 - protein, 108 aa, Artificial Sequence; identical to SEQ ID NO: 60
  source: /note=“Description of Artificial Sequence Synthetic polypeptide”

SEQ ID NO: 196 - protein, 112 aa, Artificial Sequence; identical to SEQ ID NO: 7
  source: /note=“Description of Artificial Sequence Synthetic polypeptide”

SEQ ID NO: 197 - protein, 112 aa, Artificial Sequence; identical to SEQ ID NO: 57
  source: /note=“Description of Artificial Sequence Synthetic polypeptide”

SEQ ID NO: 198 - protein, 112 aa, Artificial Sequence; identical to SEQ ID NO: 65
  source: /note=“Description of Artificial Sequence Synthetic polypeptide”

SEQ ID NO: 199 - protein, 112 aa, Artificial Sequence; identical to SEQ ID NO: 77
  source: /note=“Description of Artificial Sequence Synthetic polypeptide”

SEQ ID NO: 200 - protein, 112 aa, Artificial Sequence; identical to SEQ ID NO: 7
  source: /note=“Description of Artificial Sequence Synthetic polypeptide”

SEQ ID NO: 201 - protein, 112 aa, Artificial Sequence; identical to SEQ ID NO: 83
  source: /note=“Description of Artificial Sequence Synthetic polypeptide”

SEQ ID NO: 202 - protein, 112 aa, Artificial Sequence; identical to SEQ ID NO: 85
  source: /note=“Description of Artificial Sequence Synthetic polypeptide”

SEQ ID NO: 203 - protein, 112 aa, Artificial Sequence; identical to SEQ ID NO: 91
  source: /note=“Description of Artificial Sequence Synthetic polypeptide”

SEQ ID NO: 204 - protein, 112 aa, Artificial Sequence; identical to SEQ ID NO: 7
  source: /note=“Description of Artificial Sequence Synthetic polypeptide”

SEQ ID NO: 205 - protein, 108 aa, Artificial Sequence; identical to SEQ ID NO: 8
  source: /note=“Description of Artificial Sequence Synthetic polypeptide”

SEQ ID NO: 206 - protein, 112 aa, Artificial Sequence
  source: /note=“Description of Artificial Sequence Synthetic polypeptide”
DIVITDELSNPVTSGESVSISCRSSKSLLYKDGKTYLNWFLQRPGQSPQLLIYLMSTRASGVSDRFSGSGSGTDFTLEISRVKAEDVGVYYCQQFVEYPLTFGTGTKLELQR

SEQ ID NO: 207 - protein, 112 aa, Artificial Sequence; identical to SEQ ID NO: 7
  source: /note=“Description of Artificial Sequence Synthetic polypeptide”

SEQ ID NO: 208 - protein, 112 aa, Artificial Sequence; identical to SEQ ID NO: 29
  source: /note=“Description of Artificial Sequence Synthetic polypeptide”

SEQ ID NO: 209 - protein, 108 aa, Artificial Sequence; identical to SEQ ID NO: 8
  source: /note=“Description of Artificial Sequence Synthetic polypeptide”

SEQ ID NO: 210 - protein, 113 aa, Artificial Sequence; identical to SEQ ID NO: 108
  source: /note=“Description of Artificial Sequence Synthetic polypeptide”

SEQ ID NO: 211 - protein, 112 aa, Artificial Sequence; identical to SEQ ID NO: 7
  source: /note=“Description of Artificial Sequence Synthetic polypeptide”

SEQ ID NO: 212 - protein, 118 aa, Artificial Sequence; identical to SEQ ID NO: 107
  source: /note=“Description of Artificial Sequence Synthetic polypeptide”

(19) United States
(12) Patent Application Publication
(10) Pub. No.: US 2021/0139568 A1
(43) Pub. Date: May 13, 2021

(54) ANTIBODY-BASED METHODS OF DETECTING AND TREATING ALZHEIMER'S DISEASE

(71) Applicant: AXON NEUROSCIENCE SE, Larnaca (CY)

(72) Inventors: Jozef HANES, Bratislava (SK); Eva KONTSEKOVÁ, Senec (SK); Andrej KOVÁC, Bratislava (SK); Norbert ZILKA, Bratislava (SK)

(73) Assignee: Axon NeuroScience SE, Larnaca (CY)

(21) Appl. No.: 15/733,660

(22) PCT Filed: Mar. 27, 2019
(86) PCT No.: PCT/IB2019/000358

§ 371 Date: Sep. 25, 2020

Related U.S. Application Data
(60) Provisional application No. 62/703,299, filed on Jul. 25, 2018.
(60) Provisional application No. 62/664,662, filed on Apr. 30, 2018.
(60) Provisional application No. 62/649,206, filed on Mar. 28, 2018.

(51) Int. Cl. C07K 16/18; A61P 25/28

(52) U.S. Cl. CPC C07K 16/18; A61P 25/28; A61K 2039/505; C07K 2317/565; C07K 2317/34

(57) ABSTRACT

Disclosed herein are antibodies and antigen binding fragments that bind phosphorylated and dephosphorylated tau and methods of use in detecting and treating Alzheimer's disease and other tauopathies. Also included are methods for determining the stage of Alzheimer's disease in a human subject and monitoring the effectiveness of an anti-tau therapy.

This application is a U.S. national stage entry under 35 U.S.C. § 371 of international application number PCT/IB2019/000358, filed Mar. 27, 2019, which designates the U.S. and claims priority to U.S. Application No. 62/649,208 filed Mar. 28, 2018, U.S. Application No. 62/664,662 filed Apr. 30, 2018, and U.S. Application No. 62/703,299 filed Jul. 25, 2018, each of which is incorporated herein by reference in its entirety.

### FIELD

Disclosed herein are antibodies, compositions, kits, and methods for the detection, monitoring, prevention, and/or treatment of Alzheimer's disease and other tauopathies.

### SEQUENCE LISTING

The instant application contains a Sequence Listing, that has been submitted in ASCII format via EFS-Web and is hereby incorporated by reference in its entirety. Said Sequence Listing, created on Sep. 22, 2020, is titled 11634-0018-00_SL and is 192,800 bytes in size.

### BACKGROUND AND SUMMARY

Alzheimer's disease (AD) is a progressive neurodegenerative disorder that is associated with the destruction of higher brain structures, such as those involved in memory and cognition. The disease leads to deficits in cognitive function and declines in memory, learning, language, and in the ability to perform intentional and purposeful movements. AD is also accompanied by concomitant behavioral, emotional, interpersonal, and social deterioration. These cognitive and behavioral deficits render living difficult (Burns et al., Alzheimer's disease, The Lancet, vol. 360, Jul. 13, 2002). Late-stage AD patients are often unable to speak, comprehend language, and handle their own basic personal care, eventually requiring full-time care and supervision, and are often dependent on family members and nursing homes. AD is the leading cause of senile dementia, and is predicted to increase in prevalence as the proportion of elderly persons in the population grows. The total number of persons with AD is predicted to increase at least threefold between 2000 and 2050, rendering AD a world-wide public health problem (Sloane et al., The Public Health Impact of Alzheimer's Disease, 2000-2050: Potential Implication of Treatment Advances, Annu. Rev. Public Health, 23:213-31, 2002). Clinical detection, management, and treatment of AD remains largely inadequate. There is still an unmet need for effective methods to detect and treat AD.

AD has histologically been characterized pathologically by analyzing brain sections to identify the presence of extraneuronal plaques and intracellular and extracellular neurofibrillary tangles in the brain. Plaques are composed mainly of β amyloid (Aβ), whereas tangles comprise pathological forms of tau, such as tau conformers and their aggregates. The relationship between plaques and tangles and the disease process remains unclear, although studies suggest a link between amyloid and tau pathogenesis (Hardy et al., Genetic dissection of Alzheimer's disease and related dementias: amyloid and its relationship to tau, Nature Neuroscience, vol 1, No. 5, 1998; Oddo et al., Aβ Immunotherapy Leads to Clearance of Early, but Not Late, Hyperphosphorylated Tau Aggregates via the Proteasome, Neuron, 43: 321-332, 2004; Rapoport et al., 2002; Roberson, et al., Reducing Endogenous Tau Ameliorates Amyloid β-Induced Deficits in an Alzheimer's Disease Mouse Model, Science, 316:750, 2007; Shipton et al., Tau Protein Is Required for Amyloid β-Induced Impairment of Hippocampal Long-Term Potentiation, J. Neuroscience, 31(5):1688-1692, 2011). A central role for Aβ in AD pathology was initially proposed in a hypothesis called the “Aβ cascade,” wherein Aβ deposition is followed by tau phosphorylation and tangle formation, and then neuronal death (Hardy and Allsop, Amyloid deposition as the central event in the aetiology of Alzheimer's disease, TIPS, vol 12, 1991; Hardy and Selkoe, The Amyloid Hypothesis of Alzheimer's Disease: Progress and Problems on the Road to Therapeutics, Science, vol 297, 2002; for a review see, Walsh and Selkoe, Deciphering the Molecular Basis of Memory Failure in Alzheimer's Disease, Neuron, 44:181-193, 2004; also see Seabrook et al., Beyond Amyloid the Next Generation of Alzheimer's Disease Therapeutics, Molecular Intervention, 7(5), 2007).

Accordingly, therapeutic approaches for AD often focus on targeting Aβ and tau, and many studies on these targets continue today. The most advanced disease-targeting therapies undergoing clinical trials in AD patients include passive immunotherapies such as BAN2401, ADUCANUMAB, GANTENERUMAB and CRENEZUMAB for Aβ removal; C2N 8E12, RO 7105705 and B11B092 targeting tau protein; and active vaccine such as CAD106, Lu AF20513, ABvac 40, for Aβ therapy or ACI-35 and AADvac1 to target disease modified tau.

Recent work has led to a number of therapeutic approaches that directly or indirectly target the tau cascade in particular (for review articles, see, e.g. Dickey and Petrucelli, Pharmacologic reductions of total tau levels; implications for the role of microtubule dynamics in regulating tau expression, Molecular Neurodegeneration, 1:6, 2006; Schneider and Mandelkow, Tau-Based Treatment Strategies in Neurodegenerative Disease, Neurotherapeutics, 5:443-457, 2008; Zilka et al., Chaperone-like Antibodies Targeting Misfolded Tau Protein: New Vistas in the Immunotherapy of Neurodegenerative Foldopathies, Journal of Alzheimer's Disease, 15:169-177, 2008), including compounds that prevent or reverse tau aggregation (Wischik et al., Selective inhibition of Alzheimer's disease-like tau aggregation by phenothiazines, Proc. Natl. Acad. Sci. USA, 93:11213-11218, 1996; Necula et al., Cyanine Dye N744 Inhibits Tau Fibrillization by Blocking Filament Extension: Implications for the Treatment of Tauopathic Neurodegenerative Diseases, Biochemistry, 44:10227-10237, 2005; Pickhardt et al., Screening for Inhibitors of Tau Polymerization, Current Alzheimer Research, 2:219-226, 2005; Taniguchi et al., Inhibition of Heparin-induced Tau Filament Formation by Phenothiazines, Polyphenols, and Porphyrins, The Journal of Biological Chemistry, 280:9, 7614-7623, 2005; Larbig et al., Screening for Inhibitors of Tau Protein Aggregation into Alzheimer Paired Helical Filaments: A Ligand Based Approach Results in Successful Scaffold Hopping, Current Alzheimer Research, 4:315-323, 2007) small-molecule type drugs that inhibit tau kinases or activate tau phosphatases (Iqbal and Grundke-Iqbal, Inhibition of Neurofibrillary Degeneration: A Promising Approach to Alzheimer's Disease and Other Tauopathies, Current Drug Targets, 5:495-502, 2004; Noble et al., Inhibition of glycogen synthase kinase-3 by lithium correlates with reduced tauopathy and degeneration in vivo, PNAS, 102:19, 6990-6995, 2005; Iqbal and Grundke-Iqbal, Developing pharmacological therapies for Alzheimer disease, Cell. Mol. Life Sci., 64:2234-2244, 2007), microtubule stabilizing drugs (Zhang et al., Microtubule-binding drugs offset tau sequestration by stabilizing microtubules and reversing fast axonal transport deficits in a tauopathy model, PNAS, 102(1): 227-231, 2005), drugs that facilitate the proteolytic degradation of misfolded tau proteins (Dickey et al., Development of a High Throughput Drug Screening Assay for the Detection of Changes in Tau Levels-Proof of Concept with HSP90 inhibitors, Current Alzheimer Research, 2:231-238, 2005, Dickey et al., Pharmacologic reductions of total tau levels; implications for the role of microtubule dynamics in regulating tau expression, Molecular Neurodegeneration, 1:6, 2006), and immunosuppresive drugs (Zilka et al., Chaperon-like Antibodies Targeting Misfolded Tau Protein: New Vistas in the Immunotherapy of Neurodegenerative Foldopathies, Journal of Alzheimer's Disease, 15:169-177, 2008), as well as immunotherapeutic strategies including active and passive immunization (Schneider and Mandelkow et al., Tau-Based Treatment Strategies in Neurodegenerative Diseases, Neurotherapeutics, 5:443-457, 2008; Zilka et al., Chaperon-like Antibodies Targeting Misfolded Tau Protein: New Vistas in the Immunotherapy of Neurodegenerative Foldopathies, Journal of Alzheimer's Disease, 15:169-177, 2008, Tabira, T. Immunization Therapy for Alzheimer disease: A Comprehensive Review of Active Immunization Strategies. Tohoku J. Exp. Med., 220: 95-106 (2010)). In addition, researchers have focused on novel monoclonal antibody (mAbs) therapies to treat AD. See, e.g., Citron et al., Alzheimer's disease: strategies for disease modification, Nature Reviews, 9:397, 2010, and Asuni et al., Immunotherapy Targeting Pathological Tau Conformers In a Tangle Mouse Model Reduces Brain Pathology with Associated Functional Improvements, The Journal of Neuroscience, 27(34):9115-9129, 2007, for review. Therapeutic antibodies targeting disease forms of tau represent a promising approach for treatment and/or diagnosis of AD and other tauopathies (WO 2004/007547, US2008/0050383).

Despite this growing body of research on therapeutic strategies for treating AD and other tauopathies, there remains an unmet need for diagnostic tools that can accurately detect and distinguish AD and other tauopathies from other brain pathologies in patients presenting with dementia in order to ensure appropriately targeted treatment decisions are made, particularly at early stages of the disease processes (Blennow and Hampel, CSF markers for incipient Alzheimer's disease, Lancet Neurol., 2:605-613, 2003; Blennow, CSF markers for incipient Alzheimer's disease, Lancet Neurol., 2:605-613, 2005). Significant efforts have been made in the last two decades to identify in vivo brain indicators and sample-based biomarkers for preclinical AD, all without much success. Research has focused mainly on cerebrospinal fluid (CSF) and blood, but no definitive markers have been found that accurately detect and distinguish different types of dementia. Several CSF and blood biomarkers, for example, have been extensively studied without adequate improvements in detection accuracy. These include biomarkers of neurodegeneration (t-tau, NFL, NSE, VLP-1), APP metabolism (Aβ42, Aβ40, Aβ38, sAPPα, and sAPPβ), tangle pathology (p-tau), and glial activation (YKL-40, MCP-1, and GFAP) (Olsson et al., CSF and blood biomarkers for the diagnosis of Alzheimer's disease: a systematic review and meta-analysis, Lancet Neurol., 7:673-84, 2016). Three CSF biomarkers have been reported for use in prodromal mild cognitive impairment (MCI and AD dementia: CSF Aβ1-42 (Aβ1-42), also expressed as Aβ1-42: Aβ1-40 ratio, t-tau, and p-tau Thr181 & Thr231 proteins (Cavedo et al., The Road Ahead to Cure Alzheimer's Disease: Development of Biological Markers and Neuroimaging Methods for Prevention Trials Across all Stages and Target Populations, J. Prev. Alzheimer's Dis., 3:181-202, 2014). Nevertheless, accurate detection of early stage AD and methods to distinguish AD from other tauopathies or other causes of dementia in patients remains a challenge, particularly for CSF based assays.

Research has suggested that different phosphorylated epitopes of tau, including threonine 181, threonine 181 and 231, threonine 231, threonine 231 and 235, serine 199, serine 396 and 404, may correlate with AD, although with some contradictory reports. (Andreasen et al., Cerebrospinal fluid levels of total-tau, phospho-tau and A beta 42 predicts development of Alzheimer's disease in patients with mild cognitive impairment, Acta Neurol. Scand. Suppl., 179:47-51, 2003; Formichi et al., Cerebrospinal fluid tau, A beta, and phosphorylated tau protein for the diagnosis of Alzheimer's disease, J. Cell Physiol., 208:39-46, 2006; Blennow and Hampel, CSF markers for incipient Alzheimer's disease, Lancet Neurol., 2:605-613, 2003). For instance, it has been suggested that repeated assessment of CSF p-tau181 did not provide a useful clinical biomarker because it was insensitive to disease progression over a 2-year period (Bouwman et al., Longtitudinal changes of CSF biomarkers in memory clinic patients, Neurology, 69:1006-1011, 2007). The predictive power of CSF tau and Aβ biomarkers, and their dynamics of over time, therefore remains controversial. Limited ability to even detect these biomarkers in CSF further compounds the challenges in diagnosing AD. Several studies, for instance, described longitudinal changes of Aβ42, t-tau, and p-tau levels in CSF in AD patients (Andersson et al., Neurobiol Aging, 29:1466-1473, 2008; Blomberg et al., Neurosci. Lett., 214:163-166, 1996; Arai et al., JAGS, 45:1228-31, 1997; Kanai et al., Ann. Neurol., 44:17-26; Kanai et al., Neurosci. Lett., 267: 65-68, 1999; Hampel et al., Ann Neurol., 49:545-546, 2001; Hoglund et al., Dement. Geriatr. Cogn. Disord., 19:256-265, 2005), whereas others reported no significant changes in these CSF biomarkers over time (Nishimura et al., Methods Find. Exp. Clin. Pharmacol., 20:227-235, 1998; Andreasen et al., Arch. Neurol., 56:673-680, 1999; Sunderland et al., Biol. Psychiatry, 46:750-755, 1999; Tapiola et al., Neurosci. Lett., 280:119-122, 2000; deLeon et al., Neurobiol. Aging, 27:394-401, 2006; Mollenhauer et al., J. Neural. Transm., 112:933-948, 2005; Zetterberg et al., Alzheimers Res. Ther., 5(2):9, 2007; Mattsson et al., J. Alzheimers Dis., 30(4):767-778, 2012; Toledo et al., Acta Neuropathol. 125(5):659-70, 2013). The reason why CSF biomarkers do not seem to reflect disease progression in some patients over time is not known. One possibility is that brain derived proteins are diluted in the CSF compartment (deLeon et al., Longitudinal cerebrospinal fluid tau load increases in mild cognitive impairment, Neurosci Lett., 333:183-186, 2004). Another potential explanation is that older non-demented individuals (over 65) tend to have higher p-tau levels than younger, and so elevation of the biomarker with disease progression is masked in the older non-demented individuals (Bouwman et al., CSF biomarker levels in early and late onset Alzheimer's disease, Neurobiol Aging, 30:1895-1901, 2008). These and other factors contribute to the lack of an effective CSF-based assay.

The available assays used to detect CSF biomarkers focus almost exclusively on immunoassays involving a classical ELISA format, and among them are INNOTEST hTAU, INNOTEST phospho-Tau (recognizing phospho-Threonine 181) and INNOTEST Aβ42. Specificity and sensitivity of those assays, however, are generally not sufficient to predict Alzheimer's disease, especially in its preclinical stages (Wennström et al., The Inflammatory Marker YKL-40 Is Elevated in Cerebrospinal Fluid from Patients with Alzheimer's but Not Parkinson's Disease or Dementia with Lewy Bodies, 10(8): e0135458, PlosOne, 2015; Wang et al., Analysis of Cerebrospinal Fluid and [11C]PIB PET Biomarkers for Alzheimer's Disease with Updated Protocols, 52(4):1403-13, JAD 2016). Beyond sample-based diagnostic assays, advances in molecular imaging in recent years have led to work on potential tau-specific tracers for positron emission tomography (PET). Three families of radiotracers have been developed as tau PET tracers: the aryquinoline derivatives THK5117 and THK5351, the pyrido-indole derivative AV-1451 (also known as T807 and Flortaucipir), and the phenyl/pyridinyl-butadienyl benzothiazole/benzothiazolium derivative PBB3 (Saint-Aubert et al., Tau PET imaging: present and future directions, Mol. Neurodegener., 12:19, 2017). Since tau PET tracers displayed off target binding, mostly recognizing enzyme MAO B or neuromelanin (Barrio et al., The Irony of PET Tau Probe Specificity, J. Nucl. Med., 59(1):115-116, 2018; Lemoine et al., Comparative binding properties of the tau PET tracers THK5117, THK5351, PBB3, and T807 in postmortem Alzheimer brains, Alzheimers Res. Ther., 9(1):96, 2017; Ng et al., Monoamine oxidase B inhibitor, selegiline, reduces ¹⁸F-THK5351 uptake in the human brain, Alzheimers Res. Ther., 9(1):25, 2017), 2^{nd} generation of tau PET tracers have been developed: PI-2620, MK-6240, GTP1 and R06958948 (www.alzforum.org). However, nonspecific binding to multiple tissue targets is still present in some newly developed tracers, suggesting room for improved techniques (The 12th Human Amyloid Imaging, Miami, Fla., USA 2018).

Results from several neuropathological studies have shown that the amount of tau pathology in the brain of patients is strongly correlated with the progression of AD. The pattern of anatomical localization of tau lesions may correspond to the domains of cognition affected over the course of Alzheimer's disease, and the pattern and degree of brain atrophy (Braak and Braak, Neuropathological stageing of Alzheimer-related changes, Acta Neuropathol, 82:239-59, 1991; Murray et al., Neuropathologically defined subtypes of Alzheimer's disease with distinct clinical characteristics: a retrospective study, Lancet Neurol., (9):785-96, 2011; Nelson et al., Correlation of Alzheimer disease neuropathologic changes with cognitive status: a review of the literature, J. Neuropathol. Exp. Neurol., 71:362-81, 2012). Similarly, several independent imaging studies revealed that the distribution of tau PET signal can correlated with cognitive decline (Ossenkoppele et al., Tau PET patterns mirror clinical and neuroanatomical variability in Alzheimer's disease, Brain, 139:1551-67, 2016; Bejanin et al., Tau pathology and neurodegeneration contribute to cognitive impairment in Alzheimer's disease, Brain, 140(12):3286-3300, 2017; Mattsson et al., AV-1451 and CSF T-tau and P-tau as biomarkers in Alzheimer's disease, EMBO Mol. Med., 9:1212-1223, 2017; Mattsson et al., Comparing 18F-AV-1451 with CSF t-tau and p-tau for diagnosis of Alzheimer disease, Neurology, 5:e388-e395, 2018). Accordingly, the development of improved reagents and methods for PET imaging of tau in the brain could help enhance diagnostic accuracy and monitoring of AD patients over time. While CSF tau biomarkers are primarily useful as disease state biomarker, tau PET imaging may also be useful in monitoring AD progression, such as the transition from prodromal stage to dementia (Mattsson et al., Comparing 18F-AV-1451 with CSF t-tau and p-tau for diagnosis of Alzheimer disease, Neurology, 5:e388-e395, 2018). But given the invasive and radioactive nature of brain imaging agents, a non-invasive CSF-based assay with improved accuracy and specificity for AD would be beneficial.

The currently available biomarkers, however, display several limitations in clinical use. It has been shown that CSF levels of total tau, p-tau and Aβ42 considerably overlap between diseased and control cases (Hulstaert et al., Improved discrimination of AD patients using beta-amyloid(1-42) and tau levels in CSF, Neurology, 52:1555-1562, 1999; Hu et al., Levels of nonphosphorylated and phosphorylated tau in cerebrospinal fluid Alzheimer's disease patients: an ultrasensitive bienzyme-substrate-recycle enzyme-linked immunosorbent assay, Am. J. Pathol., 160:1269-1278, 2002). Disease heterogeneity is considered to be the major cause of the overlap in CSF biomarkers (Iqbal et al., Subgroups of Alzheimer's disease based on cerebrospinal fluid molecular markers, 58:748-757, 2005). In tau PET imaging, the critical challenge for development of newly tau-directed radiotracers is to overcome nonspecific binding (Barrio et al., The Irony of PET Tau Probe Specificity, J. Nucl. Med., 59:115-116, 2018; Lemoine et al. Comparative binding properties of the tau PET tracers THK5117, THK5351, PBB3, and T807 in postmortem Alzheimer brains, Alzheimers Res. Ther., 9(1):96, 2017; Ng et al., Monoamine oxidase B inhibitor, selegiline, reduces ¹⁸F-THK5351 uptake in the human brain, Alzheimers Res. Ther., 9(1):25. 2017). Further, recent reports showed that pathological tau conformations are diverse depending on various human tauopathies, and tau ligands have differential binding affinity to the various tau pathological entities (Choi et al., Development of tau PET Imaging Ligands and their Utility in Preclinical and Clinical Studies, 52(1):24-30, 2018). Thus, another challenge is to improve the binding potency to various tau pathological lesions in order to utilize them for diagnostics of multiple human tauopathies.

The recent development of more powerful treatments targeting AD emphasizes the need to accurately detect AD at an early stage and distinguish it from other forms of dementia in patients. Effective biomarkers, if available, would also be useful for patient stratification and longitudinal monitoring of disease progression, since the relative amount of plaques and tangles may show a marked difference between AD patients at different stages in the disease. Stratification of patients based on biomarker data may also be a way to identify subgroups of patients more prone to respond to therapy. (Hampel et al., Perspective on future role of biological markers in clinical therapy trials of Alzheimer's disease: a long-range point of view beyond 2020, Biochem. Pharmacol., 88(4):426-46, 2014).

Accordingly, disclosed herein are antibodies, compositions, kits, and methods that provide for improved detection, monitoring, prevention, and/or treatment of Alzheimer's disease and other tauopathies.

In various embodiments, the present disclosure provides an antibody or antigen binding fragment thereof capable of binding tau. In some embodiments, the antibody or antigen binding fragment thereof can bind a phosphorylated epitope in tau. In some embodiments, tau species with that phosphorylated epitope is present in a sample (e.g., blood or CSF) from a patient with AD in a greater concentration than in a patient with another tauopathy or in a healthy subject.

In some embodiments, the antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the heavy chain variable region comprises an amino acids sequence of SEQ ID NO: 7 and the light chain variable region comprises an amino acid sequence of SEQ ID NO 8.

In various embodiments, an antibody or antigen binding fragment thereof disclosed herein can bind to an epitope on tau protein 2N4R (SEQ ID NO: 9), wherein the epitope is phosphorylated. The epitope may comprise one or more of residues 188-227 of tau protein 2N4R (SEQ ID NO: 10). In some embodiments, the epitope comprises one or more of residues 210-221 of tau protein 2N4R (SEQ ID NO: 11). In certain embodiments, the epitope comprises at least one phosphorylated residue. In certain embodiments, the epitope comprises more than one phosphorylated residue. The phosphorylated residue(s) may comprise a phospho-threonine at position 217 of tau protein 2N4R (SEQ ID NO: 9). In some embodiments, the epitope also comprises a phosphorylated serine at position 210, threonine at position 212, serine at position 214, or threonine at position 220 of tau protein 2N4R, or any combination thereof. The antibody or antigen binding fragment thereof may bind to an epitope comprising or consisting of SRTPSLPpTPPTR (SEQ ID NO: 12).

In various embodiments, an antibody or antigen binding fragment thereof disclosed herein can bind to an epitope on tau comprising one or more of residues 188-227 of tau protein 2N4R (SEQ ID NO: 10). In some embodiments, the epitope comprises one or more of residues 210-221 of tau protein 2N4R (SEQ ID NO: 11). In certain embodiments, the epitope comprises at least one phosphorylated residue, wherein the at least one phosphorylated residue may be a phospho-threonine at position 217 of tau protein 2N4R (SEQ ID NO: 9). In some embodiments, the epitope also comprises a phosphorylated serine at position 210, threonine at position 212, serine at position 214, or threonine at position 220 of tau protein 2N4R, or any combination thereof. In some embodiments, the antibody or antigen binding fragment thereof can bind to an epitope comprising or consisting of SRTPSLPpTPPTR (SEQ ID NO: 12).

In various embodiments, an antibody or antigen binding fragment thereof disclosed herein can bind to an epitope on tau comprising one or more of residues 151-188 of tau protein 2N4R (SEQ ID NO: 13). In certain embodiments, the antibody or antigen binding fragment thereof may can bind to an epitope comprising one or more of residues 163-172 of tau protein 2N4R (SEQ ID NO: 14). The epitope may comprise KGQANATRIP (sequence of SEQ ID NO: 14). In another embodiment, the antibody is DC2E7 or an antigen binding fragment thereof, wherein DC2E7 is an antibody produced by a hybridoma deposited under American Type Culture Collection Patent Deposit No. PTA-124992.

In another embodiment, disclosed herein is antibody DC2E2 or an antigen binding fragment thereof, wherein DC2E2 is an antibody produced by a hybridoma deposited under American Type Culture Collection Patent Deposit No. PTA-124991.

In various embodiments, an antibody or antigen binding fragment disclosed herein is conjugated to a second agent. In some embodiments, the agent is at least one detectable label or at least one therapeutic agent for AD or another tauopathy. In certain embodiments, the agent is a radiolabel.

Also disclosed herein are methods of treating, delaying progression, or preventing the progression of AD or another tauopathy in a subject. In some embodiments, the method comprises administering to the subject an effective amount of at least one antibody according to any one of previously disclosed embodiments. In some embodiments, the method comprises detecting AD using one or more of the disclosed antibodies or antigen binding fragments before administering or recommending administration of a suitable AD treatment. In some embodiments, the method comprises administering an AD treatment to a patient who has been diagnosed as having AD using an antibody or antigen binding fragment disclosed herein.

In various embodiments, a method of detecting a tauopathy in a subject comprises: obtaining a biological sample from the subject; contacting the sample from the subject with an effective amount of a molecule that is capable of forming a complex with tau (e.g., using at least one antibody or antigen binding fragment disclosed herein that is capable of binding tau to form a tau-antibody complex); detecting the presence and/or amount of the tau-molecule complex using an antibody or antigen binding fragment described herein, wherein the presence and/or amount of tau-molecule complex indicates a tauopathy in the subject. In some embodiments, the molecule that forms a tau-molecule complex is a first antibody that can bind tau to form a tau-antibody complex, and wherein the presence of the tau-antibody complex is detected using a second anti-tau antibody or antigen binding fragment, which can bind a different epitope on tau than the first antibody. In certain embodiments, the first or second antibody or antigen binding fragment is linked (e.g., covalently or non-covalently coated on) to a solid surface or particle. In some embodiments, the first or second antibody is conjugated to a detectable label. The disclosed method may comprise a classic ELISA, digital ELISA assay, or other ELISA assay formats, e.g., using any one or more of the antibodies or fragments disclosed herein that bind to phosphorylated tau, and may detect the presence and/or amount of phosphorylated tau in the sample, wherein an increased level of phosphorylated tau in the sample indicates the subject has Alzheimer's disease rather than another tauopathy. In certain embodiments, the biological sample is cerebrospinal fluid. In certain embodiments, the biological sample is serum and/or plasma.

In various embodiments, a method of distinguishing Alzheimer's disease from another tauopathy or another cause of dementia in a subject comprises: obtaining a cerebrospinal fluid or blood sample from a subject, contacting the sample with an anti-tau antibody or antigen binding fragment thereof disclosed herein, and detecting the presence and/or amount of phosphorylated tau complexed with the antibody or antigen binding fragment in the same sample, wherein the presence and/or an elevated level of phosphorylated tau in the sample relative to the level in a sample from a healthy control subject or wherein an elevated level of phosphorylated tau above a threshold indicates the subject has Alzheimer's disease rather than another tauopathy or an alternative cause (i.e., another form of) dementia or another neurodegenerative disorder. In some embodiments, the anti-tau antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6 that is capable of binding to phosphorylated tau to form a phosphorylated tau-antibody complex. In some embodiments, the antibody or antigen binding fragment comprises a heavy chain variable region and a light chain variable region, wherein the heavy chain variable region comprises amino acid sequence of SEQ ID NO: 7 and the light chain variable region comprises an amino acid sequence of SEQ ID NO:8.

In various embodiments, a method of detecting Alzheimer's disease (AD) or mild cognitive impairment (MCI) in a subject comprises: contacting a biological sample from the subject with an effective amount of a molecule that is capable of forming a complex with tau (e.g., using at least one antibody or antigen binding fragment disclosed herein that is capable of binding tau to form a tau-antibody complex); detecting the presence and/or amount of the tau-antibody complex; and comparing the presence/amount of tau bound to the antibody in the sample to the amount in a control sample or a threshold, wherein the presence and/or an increased amount of tau complexed with the antibody relative to the control sample or threshold indicates AD or MCI in the subject. In some embodiments, MCI is a precursor of AD in a patient. In some embodiments, the method distinguishes MCI and/or AD from other neurological diseases. In some embodiments the other neurological diseases are selected from Parkinson's disease, Multiple sclerosis, amyotrophic lateral sclerosis, and/or frontotemporal dementia. In some embodiments, the biological sample comprises cerebrospinal fluid (CSF). In some embodiments, the biological sample comprises blood. In some embodiments, the biological sample comprises plasma and/or serum fractions. In some embodiments, the threshold is about 9.3 pg/ml of tau or about 5.3 pg/ml of tau. In some embodiments, the threshold is between about 100-600 pg/ml. In some embodiments, the threshold is about 300 pg/ml.

In various embodiments, a method of detecting Alzheimer's disease (AD) or mild cognitive impairment (MCI) in a subject comprises: obtaining a biological sample; detecting the presence and/or amount of tau protein 2N4R phosphorylated at least at position threonine 217 in the biological sample; and comparing the presence/amount of tau protein 2N4R phosphorylated at threonine 217 to the amount in a control sample or a threshold, wherein the presence and/or an increased amount of tau protein 2N4R phosphorylated at threonine 217 relative to the control sample or threshold indicates AD or MCI in the subject. In various embodiments, the MCI is a precursor of AD in a patient. In some embodiments, the method distinguishes MCI and/or AD from other neurological diseases. In some embodiments, the other neurological disease is selected from Parkinson's disease, Multiple sclerosis, amyotrophic lateral sclerosis, and/or frontotemporal dementia. In some embodiments, the biological sample comprises cerebrospinal fluid (CSF). In some embodiments, the biological sample comprises blood. In some embodiments, the biological sample comprises plasma and/or serum fractions. In some embodiments, the threshold is about 9.3 pg/ml of tau. In some embodiments, the threshold is about 9.3 pg/ml of tau or about 5.3 pg/ml of tau. In some embodiments, the threshold is between about 100-600 pg/ml. In some embodiments, the threshold is about 300 pg/ml.

In various embodiments, a method of distinguishing Alzheimer's disease and/or mild cognitive impairment from Parkinson's disease, multiple sclerosis, amyotrophic lateral sclerosis, and/or frontotemporal dementia in a subject comprises: contacting a biological sample from the subject with an effective amount of a molecule that is capable of forming a complex with tau (e.g., using at least one antibody or antigen binding fragment disclosed herein that is capable of binding tau to form a tau-antibody complex); and detecting the presence and/or amount of tau complexed with the antibody or antigen binding fragment in the same sample; and comparing the presence/amount of tau bound to the antibody in the sample to the amount in a control sample or a threshold, wherein the presense and/or an increased amount of tau complexed with the antibody relative to the control sample or threshold indicates Alzheimer's disease and/or mild cognitive impairment (MCI) in the subject. In some embodiments, the biological sample comprises cerebrospinal fluid (CSF). In some embodiments, the biological sample comprises blood. In some embodiments, the biological sample comprises plasma and/or serum fractions. In some embodiments, the threshold is about 9.3 pg/ml of tau. In some embodiments, the threshold is between about 100-600 pg/ml. In some embodiments, the threshold is about 300 pg/ml.

In various embodiments, a method of distinguishing Alzheimer's disease and/or mild cognitive impairment from Parkinson's disease, multiple sclerosis, amyotrophic lateral sclerosis, and/or frontotemporal dementia in a subject comprises: obtaining a biological sample from the subject; detecting the presence and/or amount of tau protein 2N4R phosphorylated at least at position threonine 217 in the biological sample; comparing the presence/amount of tau protein 2N4R phosphorylated at threonine 217 to the amount in a control sample or a threshold, wherein the presence and/or an increased amount of tau protein 2N4R phoshphorylated at threonine 217 relative to the control sample or threshold indicates Alzheimer's disease or mild cognitive impairment in the subject. In some embodiments, the biological sample comprises cerebrospinal fluid (CSF). In some embodiments, the biological sample comprises blood. In some embodiments, the biological sample comprises plasma and/or serum fractions. In some embodiments, the threshold is about 9.3 pg/ml of tau. In some embodiments, the threshold is about 9.3 pg/ml of tau or about 5.3 pg/ml. In some embodiments, the threshold is between about 100-600 pg/ml. In some embodiments, the threshold is about 300 pg/ml.

In various embodiments, a method of predicting the likelihood that a patient with mild cognitive impairment will develop Alzheimer's disease comprises: contacting a biological sample from the subject with an effective amount of a molecule that is capable of forming a complex with tau (e.g., using at least one antibody or antigen binding fragment disclosed herein that is capable of binding tau to form a tau-antibody complex); and detecting the presence and/or amount of tau complexed with the antibody or antigen binding fragment in the same sample; and comparing the presence/amount of tau bound to the antibody in the sample to the amount in a control sample or threshold, wherein the presense and/or an increased amount of tau complexed with the antibody relative to the control sample or threshold indicates an increased likelihood that the patient will develop Alzheimer's disease. In some embodiments, the biological sample comprises cerebrospinal fluid (CSF). In some embodiments, the biological sample comprises blood. In some embodiments, the biological sample comprises plasma and/or serum fractions. In some embodiments, the threshold is about 9.3 pg/ml of tau. In some embodiments, the threshold is between about 100-600 pg/ml. In some embodiments, the threshold is about 300 pg/ml.

In various embodiments, a method of predicting the likelihood that a patient with mild cognitive impairment will develop Alzheimer's disease comprises: obtaining a biological sample from the subject; detecting the presence and/or amount of tau protein 2N4R phosphorylated at least at position threonine 217 in the biological sample; comparing the presence/amount of tau protein 2N4R phosphorylated at threonine 217 to the amount in a control sample or a threshold, wherein the presence and/or an increased amount of tau protein 2N4R phoshphorylated at threonine 217 relative to the control sample or threshold indicates an increased likelihood that the patient will develop Alzheimer's disease. In some embodiments, the biological sample comprises cerebrospinal fluid (CSF). In some embodiments, the biological sample comprises blood. In some embodiments, the biological sample comprises plasma and/or serum fractions. In some embodiments, the threshold is about 9.3 pg/ml of tau. In some embodiments, the threshold is between about 100-600 pg/ml. In some embodiments, the threshold is about 300 pg/ml.

### BRIEF DESCRIPTION OF THE DRAWINGS

The accompanying drawings, which are incorporated in and constitute a part of this specification, illustrate several non-limiting embodiments of the invention and together with the description, serve to explain the principles of the disclosure.

FIG. 1. Isotype determination for monoclonal antibody DC2E2 using ELISA.

FIGS. 2A-2D. Epitope mapping of antibody DC2E2 using tau deletion mutants and tau peptides in ELISA. (FIG. 2A) Schematic illustration of tau deletion mutants and peptides used to evaluate the DC2E2 binding site on tau protein. (FIG. 2B) Immunoreactivity of DC2E2 to human six isoforms by ELISA. (FIG. 2C) Determination of DC2E2 binding site using tau deletion mutants by ELISA. (FIG. 2D) Determination of DC2E2 binding site using tau derived peptides by competitive ELISA.

FIG. 3. Immunoreactivity of DC2E2 to different tau proteins. Lane 1: sarcosyl-insoluble tau isolated from human Alzheimer's disease brain tissue; lane 2: tau151-391; lane 3: phosphorylated tau151-391, lane 4: tau 2N4R; lane 5: phosphorylated tau 2N4R.

FIG. 4. Isotype determination of monoclonal antibody DC2E7 using ELISA.

FIG. 5A-D. The nucleotide and amino-acid sequences of the variable regions in antibody DC2E2. FIG. 5A shows the nucleotide sequence encoding the light chain variable region. FIG. 5B shows the amino acid sequence of the light chain variable region, with the CDR sequences shown in bold and underlined. FIG. 5C shows the nucleotide sequence encoding the heavy chain variable region. FIG. 5D shows the amino acid sequence of the heavy chain variable region, with CDR sequences in bold and underlined. Complementarity determining regions (CDRs) were identified according to the IMGT numbering system.

FIG. 6A-D: The nucleotide and amino-acid sequences of the variable regions in antibody DC2E7. FIG. 6A shows the nucleotide sequence encoding the light chain variable region. FIG. 6B shows the amino acid sequence of the light chain variable region, with CDR sequences in bold and underlined. FIG. 6C shows the nucleotide sequence encoding the heavy chain variable region. FIG. 6D shows the amino acid sequence of the heavy chain variable region, with CDR sequences in bold and underlined. Complementarity determining regions (CDRs) were identified according to the IMGT numbering system.

FIG. 7. Immunoreactivity of DC2E7 to different tau proteins. Lane 1: tau 2N4R; lane 2: 2N4R in vitro phosphorylated; lane 3: fetal tau; lane 4: sarcosyl-insoluble tau isolated from human Alzheimer's disease brain tissue. DC25, a pan tau monoclonal antibody recognizing all forms of tau proteins, was used as control.

FIG. 8. Schematic illustration of tau deletion mutants used for evaluating the DC2E7 binding site on tau protein.

FIG. 9. Determination of DC2E7 binding site on tau protein by immunoblotting using tau deletion mutants.

FIG. 10. Schematic illustration of potential phosphorylation sites on tau in region 188-227.

FIG. 11. Determination of DC2E7 binding site on tau protein by immunobloting using tau point mutants. Mab DC25 was used as a control to measure the extent of phosphorylation of point mutations.

FIG. 12. Determination of DC2E7 epitope on tau protein by competitive ELISA using tau-derived peptides.

FIG. 13. Binding of antibodies DC2E7 and DC2E2 in Alzheimer's disease (hippocampus CA1), FTD-Pick's disease (dentate gyrus, hippocampus), CBD (nucleus caudatus) and PSP (putamen/nucleus caudatus) brain sections. Monoclonal antibody AT8 was used as a control. Tool bar 100 μm.

FIG. 14. Binding of antibodies DC2E7 and DC2E2 in Braak stage 1, Braak stage 3, and Braak stage 6 brain sections. Tool bar 100 μm.

FIG. 15. Binding of antibodies DC2E7 and DC2E2 in brain sections from Alzheimer's disease (hippocampus CA1), Pick's bodies in FTD-Pick's disease (dentate gyrus, hippocampus), and coiled bodies and astrocytic pathology in CBD (nucleus caudatus). Tool bar 20 μm.

FIG. 16. Exemplary calibration curve for DC2E7 digital ELISA assay.

FIG. 17A-B. Spike recovery experiment using three human CSF from healthy individuals. (FIG. 17A) Estimated concentrations of spiked DC2E7 calibrator in human CSF. (FIG. 17B) Recovery in % of spiked DC2E7 calibrator in human CSF.

FIG. 18. DC2E7 digital ELISA results from control and AD samples.

FIG. 19. DC2E7 digital ELISA results from AD and other tauopathy samples.

FIG. 20: DC2E7 binding to insoluble tau species in AD and other human tauopathies.

FIG. 21: A comparison of CSF samples from AD and FTD subjects using pT217 tau and pT181 tau assays.

FIG. 22: A comparison of CSF samples from AD and control subjects using pT217 tau and pT181 tau assays.

FIG. 23A-B: A comparison of CSF samples from control, MCI, and AD subjects using pT217 tau assays (FIG. 23A). A comparison of CSF samples from control, AD, PD, MS, ALS, and FTD subjects using pT217 tau assays (FIG. 23B).

FIG. 24: Absorbance of isolated scFV antibody fragments derived from DC2E7.

FIG. 25A-D: Alignment of amino acid sequences of scFV antibody fragments derived from DC2E7. FIG. 25A shows scFV antibody light chain variable domains compared to the DC2E7 sequence. FIG. 25B shows scFV antibody heavy chain variable domains compared to the DC2E7 sequence. FIG. 25C shows the variable light chain domains of the scFV antibody fragments which exhibited higher affinity as compared to DC2E7. FIG. 25D shows the variable heavy chain domains of the scFV antibody fragments which exhibited higher affinity as compared to DC2E7. Residues identical to the sequence of DC2E7 are represented by dots. CDRs were identified according to the IMGT numbering system.

FIG. 26: Comparison of the affinity of the DC2E7 and DC149 antibodies for tau derived peptide 2E7pep.

FIG. 27A-B: Alignment of amino acid sequences of DC2E7 and DC149. FIG. 27A shows the DC149 variable light chain domain in comparison to the DC2E7 light chian sequence. FIG. 27B shows the DC149 variable heavy chain domain in comparison to the DC2E7 heavy chain sequence. Residues identical to the sequence of DC2E7 are represented by dots. CDRs were identified according to the IMGT numbering system.

FIG. 28A-B: Alignment of amino acid sequences of DC2E7 and DC807. FIG. 28A shows the DC807 variable light chain domain in comparison to the DC2E7 light chain sequence. FIG. 28B shows the DC807 variable heavy chain domain in comparison to the DC2E7 heavy chain sequence. Residues identical to the sequence of DC2E7 are represented by dots. CDRs were identified according to the IMGT numbering system.

FIG. 29: The distribution of pT217 tau as measured by a pT217 tau digital ELISA assay in samples from Alzheimer's disease, other tauopathies, and control individuals using a phosphorylated tau calibrator (left panel) and 2E7 peptide calibrator (2E7pep) (right panel).

### DETAILED DESCRIPTION

### Definitions

In order to better understand the disclosure, certain definitions are provided first.

The term “affinity” refers to the strength of the sum total of noncovalent interactions between a single binding site of a molecule (e.g., an antibody) and its binding partner (e.g., an antigen). The affinity of a molecule X for its partner Y can generally be represented by the equilibrium dissociation constant (K_{D}) (or its inverse equilibrium association constant, KA). Affinity can be measured by common methods known in the art, including those described herein. See, for example, Pope M. E., Soste M. V., Eyford B. A., Anderson N. L., Pearson T. W., (2009) J. Immunol. Methods. 341(1-2):86-96 and methods described herein.

The term “amino acid” refers to naturally occurring, modified, and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and 0-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Suitable amino acids include, without limitation, both D- and L-isomers of the 20 common naturally occurring amino acids found in peptides as well as the naturally occurring and unnaturally occurring amino acids prepared by organic synthesis or other metabolic routes. Examples of such unnaturally occurring amino acids include, but are not limited to, N-acetylglucosaminyl-L-serine, N-acetylglucosaminyl-L-threonine, and 0-phosphotyrosine. Modified amino acids include, but are not limited to, hydroxyproline, pyroglutamate, gamma-carboxyglutamate, 0-phosphoserine, azetidinecarboxylic acid, 2-aminoadipic acid, 3-aminoadipic acid, beta-alanine, aminoproprionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminoproprionic acid, N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline. The term amino acid also includes naturally occurring amino acids that are metabolites in certain organisms but are not encoded by the genetic code for incorporation into proteins. Such amino acids include, but are not limited to, ornithine, D-ornithine, and D-arginine.

The term “antibody” refers to an immunoglobulin, whether genetically engineered, natural, or wholly or partially synthetically or recombinantly produced. Intact antibodies typically comprise a heavy chain and a light chain, each comprised of a variable domain forming the binding pocket for an antigen and a constant domain that contributes to effector function. The antibody, by virtue of its chosen heavy chain, can be a member of any immunoglobulin class and subclass, including any of the human classes: IgG, IgM, IgA, IgD, and IgE, or a derivative or fragment thereof. Likewise, the light chain of the antibody may derive from any species, such as a human kappa (κ) or lambda (λ) light chain, determined based on the amino acid sequences of the constant domain.

The basic antibody structural unit typically comprises a tetramer. In various embodiments, the tetramer comprises two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 or more amino acids. See generally, Fundamental Immunology Ch 7. (Paul, W., 2^{nd} ed. Raven Press, N.Y. (1989)) (incorporated by reference in its entirety for all purposes). The variable regions of each light/heavy chain pair form the antibody binding site. Thus, an intact antibody typically has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3, and FR4. The assignment of amino acids to each domain may be done in accordance with the IMGT numbering system. Alternative definitions are also known to know of ordinary skill in the art. See, e.g., Kabat Sequences of Proteins of Immunological Interest (National Institutes of Health, Bethesda, Md. (1987 and 1991)), or Clothia & Lesk J. Mol. Biol. 196:901-917 (1987); Clothia et al. Nature 342:878-883 (1989).

“Antibody fragment” or “antigen binding fragment” comprise a portion of a full length antibody, generally at least the antigen binding portion/domain or the variable region thereof. The term antibody fragment is a subset of the term antibody discussed above. Examples of antibody fragments or antigen binding fragments include: Fab, Fab′, F(ab′)2, Fd, scFv, (scFv)2, scFv-Fc, Fv fragment, diabodies, single-chain antibody molecules, immunotoxins, and multi-specific antibodies formed from antibody fragments. In addition, antibody fragments comprise single chain polypeptides having the characteristics of a VH chain binding pathological tau, namely being able to assemble together with a VL chain or of a VL chain binding to pathological tau, namely being able to assemble together with a VH chain to form a functional antigen binding pocket and thereby providing the property of binding to tau. The terms also comprise fragments that per se are not able to provide effector functions (e.g., Antibody-dependent cell-mediate cytotoxicity (“ADCC”) or complement dependent cytotoxicity (“CDC”) but provide this function after being combined with the appropriate antibody constant domain(s).

An antibody or binding fragment thereof “capable of binding tau,” as used herein, refers to an antibody or binding fragment that preferentially binds to tau over other antigen targets. The term is interchangable with an “anti-tau” antibody or an “antibody that binds tau.” In some embodiments, the antibody or binding fragment capable of binding to tau can do so with higher affinity for that antigen than others. In some embodiments, the antibody or binding fragment capable of binding tau can bind to that antigen with a K_{D} of at least about 10⁻¹, 10⁻², 10⁻³, 10⁻⁴, 10⁻⁵, 10⁻⁶, 10⁻⁷, 10⁻⁶, 10⁻⁹, 10⁻¹¹, 10⁻¹² or greater (or any value in between), e.g., as measured by surface plasmon resonance or other methods known to the skilled artisan.

The term “chimeric” antibodies refers to antibodies in which a portion of the heavy and/or light chain is identical with or homologous to corresponding sequences in antibodies derived from a particular species or belonging to a particular antibody class or subclass (e.g., chimeric humanized, class-switched antibodies), while the remainder of the chain(s) is identical with or homologous to corresponding sequences in antibodies derived from another species or belonging to another antibody class or subclass, as well as fragments of such antibodies, so long as they exhibit desired biological activity (U.S. Pat. No. 4,816,567; and Morrison et al., Proc. Natl. Acad. Sci. USA, 81:6851-6855 (1984)).

In one embodiment, the term “chimeric antibody” refers to a monoclonal antibody comprising a variable region from one source or species and at least a portion of a constant region derived from a different source or species, usually prepared by recombinant DNA techniques. In some embodiments, chimeric antibodies comprise a murine variable region and a human constant region. Such murine/human chimeric antibodies may be produced by expressing immunoglobulin genes comprising DNA segments encoding murine immunoglobulin variable regions and DNA segments encoding human immunoglobulin constant regions. Other forms of “chimeric antibodies” may be those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now known in the art. See, e.g., Morrison, S. L., et al., Proc. Natl. Acad Sci. USA 81 (1984) 6851-6855; U.S. Pat. Nos. 5,202,238 and 5,204,244.

“Competitive binding” may be determined in an assay in which the immunoglobulin/antibody/binding fragment under testing inhibits specific binding of a reference antibody to a common antigen, such as tau (e.g., tau SEQ ID No 12). Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immuno) 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1¹²⁵ label (see Morel et al., Mol. Immunol. 25(1):7 (1988): solid phase biotin-avidin EIA (Cheung et al., Virology 176:546 (1990)); and direct labeled RIA. (Moldenhauer et al., Scand. J. Immunol. 32:77 (1990)). In some embodiments, such an assay involves the use of purified antigen bound to a solid surface or cells bearing either of these, an unlabeled test immunoglobulin and a labeled reference immunoglobulin. Competitive inhibition may be measured by determining the amount of label bound to the solid surface or cells in the presence of the test immunoglobulin. In some embodiments, the test immunoglobulin is present in excess. Usually, when a competing antibody is present in excess, it will inhibit specific binding of a reference antibody to a common antigen by at least 50-55%, 55-60%, 60-65%, 65-70%, 70-75% or more.

The term “conjugated,” as used herein, refers to a bond or chemical moiety formed from a chemical reaction between a functional group of a first molecule (e.g., an antibody) with a functional group of a second molecule (e.g., a detectable signor or therapeutic agent or drug). Such bonds include, but are not limited to, covalent linkages and non-covalent linkages, while such chemical moieties include, but are not limited to, esters, carbonates, imines phosphate esters, hydrazones, acetals, orthoesters, peptide linkages, and oligonucleotide linkages.

“Delaying progression” and “preventing the progression” refers to administration of a therapeutic agent to a patient susceptible to, or otherwise at risk of, a particular disease, such as AD. Prevention encompasses prophylactic administration to a subject at risk for AD. Anyone in the general population is at risk for AD. Some individuals have an increased risk for AD. Some individuals have an increased, genetic risk for AD. Delaying progression and preventing the progression can eliminate or reduce the risk or delay the onset of disease. Delay of AD onset or progression can be measured by comparing to standard disease progression timelines in similar populations or individuals. See Ostrowitzki et al., Alzherimers Res Ther., 9(1):95, 2017. Specific illustrative and exemplary embodiments for delaying progression are described below.

The term “epitope” refers to a site on an antigen to which an immunoglobulin or antibody (or antigen binding fragment thereof) can specifically bind. Thus, an epitope on tau is the site on tau where an immunoglobulin or antibody (or antigen binding fragment thereof) can specifically bind. Specific binding refers, in some embodiments, to binding that is measurably different from a non-specific interaction. Specific binding can be measured, for example, by determining binding of a molecule compared to binding of a control molecule, which generally is a molecule of similar structure that does not have binding activity, or by competition assay with a control molecule that shares similar binding affinity but is unlabeled. In this case, specific binding is indicated if the binding of the labeled target to a probe is competitively inhibited by excess unlabeled target. The term can be exhibited, for example, by a molecule having a Kd for the target of at least about 10⁻⁴M, alternatively at least about 10⁻⁵M, alternatively at least about 10⁻⁶ M, alternatively at least about 10⁻⁷M, alternatively at least about 10⁻⁶M, alternatively at least about 10⁻⁹M, alternatively at least about 10⁻¹⁰M, alternatively at least about 10⁻¹¹M, alternatively at least about 10⁻¹² M, or greater. In one embodiment, the term “specific binding” refers to binding where a molecule binds to a particular polypeptide or epitope on a particular polypeptide without substantially binding to any other polypeptide or epitope.

An epitope, as the term is used herein, can be formed from either contiguous amino acids or noncontiguous amino acids juxtaposed by tertiary folding of a protein. An epitope may include additional stretches of amino acids around a core region bound by an antibody, e.g., 1, 2, 3, 4, 5, 10, 15, 20, or more amino acids on the N and/or C terminus of a core epitope peptide. Epitopes formed from contiguous amino acids are typically retained on exposure to denaturing solvents whereas epitopes formed by tertiary folding are typically lost on treatment with denaturing solvents. An epitope typically includes at least 3, 4, 5, 6, 7, 8, 9, 10, 11, 12, 13, 14, or 15 amino acids, often in a unique spatial conformation. Methods of determining spatial conformation of epitopes include, for example, alanine scanning, x-ray crystallography, and 2-dimensional nuclear magnetic resonance. See, e.g., Epitope Mapping Protocols in Methods in Molecular Biology, Vol. 66, G. E. Morris, Ed. (1996). Exemplary methods are discussed and used herein for the disclosed antibodies. A “conformational epitope” is an epitope to which the antibody or tau-binding fragment thereof binds in a conformational-specific manner. In the case of protein-based epitopes, the binding can depend on the epitope-carrying-protein's secondary, tertiary, or quaternary structure. In other words, the antibody binds in a structure specific manner, or a quaternary-structure-specific manner.

An epitope on tau, as discussed herein, is identified with reference to amino acid residues on tau protein 2N4R. One of skill in the art could readily identify corresponding amino acid residues on other tau isoforms and fragments, e.g, via alignment programs such as BLAST®. Unless otherwise indicated, a reference to a particular amino acid residue on tau refers to tau protein 2N4R and should be understood to encompass the corresponding positions on other tau isoforms and fragments.

“Fab” fragments can be produced by papain digestion of antibodies, each with a single antigen-binding site, and a residual “Fc” fragment, whose name reflects its ability to crystallize readily (fragment crystalizable). Pepsin treatment yields an F(ab′)2 fragment that has two antigen-binding sites and is still capable of cross-linking antigen. “Fv” is the variable region portion of the heavy chain that is included in the Fab fragment. Any of these fragments can also be produced recombinantly. The Fc portion of an antibody is associated with the antibody's effector functions, including antibody-dependent cellular cytotoxicity (ADCC) and complement-dependent cytotoxicity or phagocytosis. Alterations (e.g., mutations or glycosylation changes) in the Fc region of an antibody can be used to modulate any of its effector functions as well as increase its serum half-life and other pharmacokinetic properties.

The Fab fragment also contains the constant domain of the light chain and the first constant domain (CH1) of the heavy chain. Fab′ fragments typically differ from Fab fragments by the addition of a few residues at the carboxy terminus of the heavy chain CH1 domain, including one or more cysteines from the antibody hinge region. Fab′-SH is the designation herein for Fab′ in which the cysteine residue(s) of the constant domains bear at least on free thiol group. F(ab′)2 antibody fragments originally were produced as pairs of Fab′ fragments which have hinge cysteines between them. Other chemical couplings of antibody fragments are also known.

The term “Fc”, as used herein, includes the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains C gamma 2 and C gamma 3 (Cgamma2 and Cgamma3) and the hinge between C gamma 1(Cgamma1) and C gamma 2 (Cgamma2). Although the boundaries of the Fc region may vary, the human IgG heavy chain Fc region is usually defined to comprise residues C226 or P230 to its carboxyl-terminus, wherein the numbering is according to the EU numbering system (Edelman G. M. et al., (1969) Proc. Natl. Acad. Sci. USA, 63(1); 78-85). The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Fc may refer to this region in isolation or this region in the context of an Fc polypeptide, for example an antibody. The Fc may be a native sequence Fc or a variant Fc. Replacements of amino acid residues in the Fc portion to alter antibody effector function are known in the art (see, e.g., Winter et al., U.S. Pat. Nos. 5,648,260 and 5,624,821). One suitable Fc, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein may exhibit reduced affinity for Fc receptors.

“Fv” refers to the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) may have the ability to recognize and bind an antigen, although at a lower affinity than the entire binding site.

As used herein, a humanized antibody that comprises a heavy or light chain variable “framework region” from a particular human germline immunoglobulin sequence may contain amino acid differences as compared to the heavy or light chain variable framework region of the particular germline sequence, due to, for example, naturally-occurring somatic mutations or intentional introduction of site-directed mutation. In various embodiments, a selected humanized antibody can be at least 90% identical in amino acid sequence of the heavy or light chain variable framework region to an amino acid sequence encoded by the heavy or light chain variable framework region of a human germline immunoglobulin gene and contains amino acid residues that identify the humanized antibody as being derived from the human when compared to the germline immunoglobulin amino acid sequences of other species (e.g., murine germline sequences). In certain cases, a humanized antibody may share at least 90%, 95%, 96%, 97%, 98%, 99% or greater amino acid sequence identity with the heavy or light chain variable framework region encoded by the germline immunoglobulin gene. In some embodiments, the heavy or light chain variable framework region of a humanized antibody derived from a particular human germline sequence will display no more than 11 amino acid, preferably no more than 5, or even more preferably no more than 4, 3, 2, or 1 differences from the amino acid sequence of the heavy or light chain variable framework region encoded by the human germline immunoglobulin gene.

“Framework Region” or “FR” residues are those variable domain residues other than the hypervariable region residues as herein defined. They bracket the hypervariable regions in the varialble domain. The FR residues can be identified according to a standard numbering system, e.g., the Kabat, Chothia, or modified Chotia numbering schemes. In the IMGT unique numbering system, the conserved amino acids always have the same position, for instance cysteine 23 (1st-CYS), tryptophan 41 (CONSERVED-TRP), hydrophobic amino acid 89, cysteine 104 (2^{nd}-CYS), phenylalanine or tryptophan 118 (J-PHE or J-TRP). See, e.g., Lefranc M. P., Immunology Today 18, 509 (1997); Lefranc M. P., The Immunologist, 7, 132-136 (1999); Lefranc, M. P., Pommié, C., Ruiz, M., Guidicelli, V., Foulquier, E., Truong, L., Thouvenin-Contet, V. and Lefranc, Dev. Comp. Immunol., 27, 55-77 (2003). In another embodiment, the IMGT unique numbering provides a standardized delimitation of the framework regions (FR1-IMGT: positions 1 to 26, FR2-IMGT: 39-55, FR3-IMGT: 66-104 and FR4-IMGT: 118 to 128) and of the complementarity determining regions: CDR1-IMGT: 27 to 38, CDR2-IMGT: 56 to 65 and CDR3-IMGT: 105 to 117. The IMGT unique numbering can be used in 2D graphical representations, designated as IMGT Colliers de Peries. See, e.g., Ruiz, M. and Lefranc, M. P., Immunogenetics, 53, 857-883 (2002); Kaas, Q. and Lefranc, M. P., Current Bioinformatics, 2, 21-30 (2007). It may also used for representing 3D structures. See, e.g., IMGT/3Dstructure-DB Kaas, Q., Ruiz, M. and Lefranc, M. P., T cell receptor and MHC structural data. Nucl. Acids. Res., 32, D208-D210 (2004).

The term “hinge” or “hinge region” or “antibody hinge region” herein includes the flexible polypeptide comprising the amino acids between the first and second constant domains of an antibody or tau-binding fragment thereof. The “hinge region” as referred to herein may be a sequence of 6-62 amino acids in length, only present in IgA, IgD, and IgG, which encompasses the cysteine residues that bridge the two heavy chains.

The term “human antibody,” as used herein, is intended to include antibodies having variable and constant regions derived from human germline immunoglobulin sequences and may be, e.g., isolated from a human or produced recombinantly. The constant regions of the antibody can be, for example, the constant regions of a human IgG1 type antibody. Such regions can be allotypic and are described by, e.g., Johnson, G., and Wu, T. T., Nucleic Acids Res. 28 (2000) 214-218 and the databases referenced therein.

The term “humanized antibody” refers to antibodies in which the framework regions (FR) and/or the complementarity determining regions (CDR) have been modified to comprise amino acid residues of an immunoglobulin from a human as compared to that of the parent immunoglobulin (e.g., parent mouse immunoglobulin residues). In one embodiment, a murine CDR is grafted into the framework region of a human antibody to prepare the “humanized antibody.” In another embodiment, human frameworks are “grafted” or spliced into mouse antibodies, preserving the CDRs of the mouse antibody and replacing its frameworks with frameworks of human origin. Grafting and splicing can be done by various recombinant DNA technologies, including PCR and mutagenesis. Various humanization methods exist in the art (e.g., CDR grafting, reshaping, transgenic animals, combinatorial libraries). See, e.g., Riechmann, L., et al., Nature 332 (1988) 323-327; Neuberger, M. S., et al., Nature 314 (1985) 268-270; Sastry L, Alting-Mess M, Huse W D, Short J M, Sorge J A, Hay B N, Janda K D, Benkoviv S J, Lerner R A (1989) Cloning of the immunological repertoire for generation of monoclonal catalytic antibodies: construction of a heavy chain variable region-specific cDNA library. Proc Nati Acad Sci USA 86, 5728-5732; and Huse W D, Sastry S, Iverson S A, Kang A S, Alting-Mees M, Burton D R, Benkoviv S J, Lerner R A (1989) Generation of a large combinatorial library of the immunoglobulin repertoire in phage lambda. Science 246, 1275-1281. In some embodiments, humanized antibodies are more human-like while retaining their original antigen-binding properties. Presta, L. G. Engineering of therapeutic antibodies to minimize immunogenicity and optimize function. Advanced Drug Delivery Reviews, Volume 58, Issues 5-6: 640-656 (2006). In some embodiments, the sequence of the variable domain of a rodent antibody is screened against a library of known human variable-domain sequences or a library of human germline sequences. The human sequence that is closest to that of the rodent may then be accepted as the human framework region for the humanized antibody (Sims et al., J. Immunol. 1993; 151:2296 et seq.; Chothia et al, Chothia and Lesk, J. Mol. Biol. 1987; 196901-917). In another embodiment, humanization involves using a particular framework region derived from the consensus sequence of all human antibodies of a particular subgroup of light or heavy chains. The same framework may be used for several different humanized antibodies (Carter et al., PNAS USA, 1992; 89:4285 et seq.; Presta et al., J Immunol 1993; 151:2623 et seq.). Other methods designed to reduce the immunogenicity of the antibody molecule in a human patient include veneered antibodies (see, e.g., U.S. Pat. No. 6,797,492 and U.S. patent application publications 20020034765 and 20040253645) and antibodies that have been modified by T-cell epitope analysis and removal (see, e.g., U.S. patent application publications 20030153043 and U.S. Pat. No. 5,712,120). In various embodiments, CDRs of the humanized antibodies described herein correspond to the CDR sequences of the mouse monoclonal DC2E7 antibody, namely SEQ ID Nos. 1-6.

The term “hypervariable region” when used herein refers to the amino residues of an antibody that contribute most directly to antigen-binding. The term is synonymous with the term “complementarity determining region” (or “CDR”). In one embodiment, according to IMGT, the hypervariable region generally comprises amino acids in three stretches on each of the heavy and light chain variable domains (e.g., residues 27-32 (LCDR1), 49-51 (LCDR2), and 88-96 (LCDR3) in the light chain variable domain and 26-33 (HCDR1), 51-58 (HCDR2), and 97-102 (HCDR3) in the heavy chain variable domain.

The term “insoluble tau” refers to aggregates of tau (such as neurofibrillary tangles, neuropil threads, Pick's bodies, coiled bodies etc.), which may be identified, e.g., by its characteristic pattern in the brain or in solution. For example, tau aggregates can be separated by centrifugation of homogenized brain samples and evaluated by, e.g., Western blot or ELISA assays. See, e.g., Lasagna-Reeves et al., FASEB J. 26:1946-59 (2012). Tau aggregates may be composed of tau monomers, tau dimers, trimers, or oligomers, such as granular tau oligomers (GTO) and various types of filaments, including Paired Helical Filaments (PHF) and Straight Filaments (SF), among others. Cowan, C. M. and Mudher, A., Are tau aggregates toxic or protective in tauopathies?, Frontiers in Neurology, 4:114 (2013).

The term “isolated nucleic acid” as used herein means a polynucleotide of genomic, cDNA, or synthetic origin or some combination thereof, which by virtue of its origin either (1) is not associated with all or a portion of a polynucleotide in which the “isolated nucleic acid” is found in nature, (2) is operably linked to a polynucleotide which is not liked to in nature, or (3) does not occur in nature as part of a larger sequence.

“Linked” refers to attachment of a moiety to a peptide, antibody, compound, or solid particle. The term embraces instances where a moiety is coupled, or complexed, or covalently or non-covalently attached to a peptide, antibody, compound, or solid particle. For example, an antibody or antigen binding fragment disclosed herein can be covalently or non-covalently coated on a bead or other sold particle. The moiety can be chemically crosslinked or expressed or synthesized as a fusion with the peptide or antibody.

As used herein, a “molecule that is capable of forming a tau-molecule complex” is any agent that preferentially can bind to a tau species to form a complex. The term encompasses antibodies, e.g., the anti-tau antibodies disclosed herein, as well as antigen receptors, Fc-conjugated receptors, receptor fragments, complement factors, and any other suitable binding moieties capable of forming a complex with tau.

The term “nucleic acid” as used herein, is intended to include DNA molecules and RNA molecules. A nucleic acid molecule may be single-stranded our double stranded.

“Pathological tau” includes pathological tau conformers and structures and encompasses all of the following: disease modified tau (e.g., hyperphosphorylated tau, truncated tau, etc.), misordered tau, misdisordered tau, misdisordered soluble tau, insoluble tau, tau oligomers and filaments, extracellular and intracellular tau aggregates such as neurofibrillary tangles, neuropil threads, neuritic plaques, ghost tangles and axonal spheroids, Pick's bodies, coiled bodies, tuft-shape astrocytes, astrocytic plaques, or any other form of tau associated with AD or another tauopathy.

A “sample” or “biological sample” as used herein refers to any portion of tissue or bodily fluid (e.g., blood, CSF, etc.) obtained from a subject for testing purposes. The sample may be directly isolated from the subject or further processed prior to testing (e.g., by dilution, fixation, denaturation, division, separation, centrifugation, immune complex dissociation, and the like). Portions of the sample used for diagnostic or monitoring purpose are also encompassed by the terms. The sample may also be further processed during or after testing (e.g., by immunoprecipitation, purification, partition, etc.). The terms “sample” and “biological sample” apply equally to the tissue or bodily fluid before, during, and/or after these processing steps. The term “control sample” as used here in refers to a sample obtained from a healthy patient or a patient with another diagnosed tauopathy or a patient with a known level of a tau species in a biologic fluid, e.g., CSF.

Various types of tau (i.e., tau protein species) can exist in a subject, e.g., in brain tissue or samples such as CSF or blood. Details on these types of tau are known and have been described, e.g., in WO2004/007547 A2 and U.S. Pat. No. 9,518,101, which are incorporated herein by reference in their entirety. The term “tau,” when used without further isoform specification (e.g., tau 2N4R), encompasses all forms of tau as well as tau fragments unless otherwise specified.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

The term “phosphorylated tau” refers to any tau isoform comprising one or more phosphorylated amino acids. The longest form of adult human brain tau has 80 serine or threonine residues and 5 tyrosine residues; therefore, almost 80% of the molecule has the potential to be phosphorylated. See Goedert, M. et al, Multiple isoforms of human microtubule-associated protein tau: sequences and localization in neurofibrillary tangles of Alzheimer's disease. Neuron 3, 519-526 (1989). The term phosphorylated tau also refers to any combination of phosphorylated tau amino acids. Tau plays a key role in regulating microtubule dynamics, axonal transport and neurite outgrowth. All of these functions of tau may be modulated by site-specific phosphorylation and alteration of normal phosphorylation events (e.g., hyperphosphorylation, abnormal phosphorylation) may contribute to neurodegenerative diseases, such as AD. Johnson G V et al., Tau phosphorylation in neuronal cell function and dysfunction. J. Cell Sci. 2004 Nov. 15,117(Pt 24): 5721-9.

In some embodiments, phosphorylated tau refers to a tau protein having any combination of one or more phosphorylated residues between amino acids 188 and 227 (as numbered in tau protein 2N4R or the equivalent residues in another tau fragment or isoform). In another embodiment, phosphorylated tau refers to tau phosphorylated at least at threonine 217 (as numbered in tau protein 2N4R or the equivalent residues in another tau fragment or isoform). In some embodiments, the tau protein is phosphorylated as in the phosphorylated tau proteins detected in the Examples.

“Physiological tau” refers to a native, unfolded tau protein found in the brain of health individuals, which can be of varying length. Such tau proteins are typically highly soluble and generally show less tendency for aggregation. See Wang et al., Tau in physiology and pathology, Nature Reviews, 17:22-35, 2016.

The term “recombinant host cell” (or simply “host cell”), as used herein, is intended to refer to a cell into which a recombinant expression vector has been introduced. It should be understood that such terms are intended to refer to not only the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein “specifically binds” in reference to an antibody means that the antibody binds to its target antigen or epitope with greater affinity than it does to a structurally different antigen(s) or epitope.

“Single-chain Fv” or “scFv” antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “treatment” as used herein, is defined as the application or administration of a therapeutic agent to a subject, who has a disease, a symptom of disease or a predisposition toward a disease, with the purpose to cure, heal, alleviate, relieve, alter, remedy, ameliorate, improve, or affect the disease, one or more symptoms of the disease, or the predisposition toward the disease. Moreover, as long as the compositions of the disclosure either alone or in combination with another therapeutic agent cure, heal, alleviate, relive, alter, remedy, ameliorate, improve or affect at least one symptom of Alzheimer's Disease or another tauopathy being treated, as compared to that symptom in the absence of treatment, the result should be considered a treatment of the underlying disorder regardless of whether all the symptoms of the disorder are cured, healed, alleviated, relieved, altered, remedied, ameliorated, improved or affected or not. Treatment may be achieved using an “effective amount” of a therapeutic agent, which shall be understood to embrace partial and complete treatment, e.g., partial or complete curing, healing, alleviating, relieving, altering, remedying, ameliorating, improving, or affecting the disease, one or more symptoms of the disease, or the predisposition toward the disease. An “effective amount” of may be determined empirically. Likewise, a “therapeutically effective amount” is a concentration or which is effective for achieving a stated therapeutic effect.

“Tauopathy” refers to a disease associated with the formation of pathological tau. Tauopathy encompasses all neurological diseases that are accompanied by the appearance of abnormal forms of microtubule associated protein tau in the brains of patients. The term includes, but is not limited to, the following diseases: Alzheimer's disease, Gerstmann-Sträussler-Scheinker disease, British dementia, Danish dementia, Pick's disease, Progressive supranuclear palsy, Corticobasal degeneration, Argyophilic grain disease, Guam Parkinsonism-dementia complex, Tangle-only dementia, White matter tauopathy with globular glial inclusions, Frontotemporal dementia (e.g., FTDP-17), Parkinsonism linked to chromosome 17 chronic traumatic encephalopathy, and nodding disease. See, e.g., Goedert M, Clavaguera F and Tolnay M. The propagation of prion-like protein inclusions in neurodegenerative diseases. Trends Neurol. Sci. In some embodiments, a subject with Frontotemporal dementia (FTD) may have Nonfluent/Agrammatic Primary Progressive Aphasia (nfPPA), Semantic Variant Primary Progressive Aphasia (svPPA), Behavioral Variant Frontotemporal Dementia (bvFTD), or Amyotrophic Lateral Sclerosis/Frontotemporal Dementia (ALS/FTD). In one embodiment, one or more of those abnormal forms of tau is recognized by one of the antibodies or binding fragments described herein in at least one assay. In some embodiments, the assay is IHC. In other embodiments, the assay is ELISA. The term “another tauopathy” encompasses all neurological diseases (e.g., tau-based causes of dementia) other than AD that are accompanied by the appearance of pathologic tau, e.g., any of the other taupathies listed above. In contrast, dementia may also arise from non-tau based etiologies, and these would fall outside of the term tauopathy.

The term “variable domain” refers to the fact that certain portions of an immunoglobulin differ extensively in sequence among antibodies and are used in the binding and specificity of each particular antibody for its particular antigen. However, the variability is often not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, which often adopt a beta-sheet configuration, connected by three hypervariable regions, which often form loops connecting, and in some cases forming part of, the beta-sheet structure. The hypervariable regions (HVR) in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md. (1991)). The “constant domains” of an antibody, in contrast, are not typically involved directly in binding an antibody to an antigen, but contribute to antibody dependent cellular cytotoxicity (ADCC).

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is most commonly used form of vector. However, the invention is intended to include other forms of expression vectors, such as viral vectors (e.g., replication defective retroviruses, adenoviruses and adeno-associated viruses), which serve equivalent functions.

As used herein, the singular forms “a” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” As used herein, the terms “about” and “approximately” mean within an acceptable error range for the particular value as determined by one of ordinary skill in the art, which will depend in part on how the value is measured or determined, i.e., the limitations of the measurement system. For example, “about” can mean from 1 to 1.5 standard deviation(s) or from 1 to 2 standard deviations, per the practice in the art. Alternatively, “about” can mean a range of up to and including 20%, 10%, 5%, or 1% of a given value. Alternatively, particularly with respect to biological systems or processes, the term can mean up to and including an order of magnitude, up to and including 5-fold, and up to and including 2-fold, of a value. Where particular values are described in the application and claims, unless otherwise stated the term “about” meaning within an acceptable error range for the particular value should be assumed. Also, all ranges described herein include the endpoints as well as all points in between. The term “or” will be understood to mean “and/or” unless the context clearly indicates otherwise. All references cited herein are incorporated by reference in their entirety. To the extent anything in an incorporated reference contradicts or is inconsistent with material provided herein, the present disclosure shall control.

### Antibodies

Described herein are novel anti-tau antibodies useful for detecting, monitoring, treating, and preventing AD. In some embodiments, the antibodies are capable of binding to a phosphorylated tau. In some embodiments, the antibodies are capable of binding to phosphorylated tau in a biological sample (e.g., CSF or blood), making them useful for non-invasively distinguishing AD from other tauopathies. In some embodiments, the antibodies can be conjugated to second agents (e.g., agents other than antibodies or antigen binding fragments disclosed herein, such as radiotracers or therapeutic agents). In some embodiments, one or more of the antibodies or antigen binding fragments disclosed herein are conjugated to each other. In some embodiments, the antibodies and/or conjugated antibodies cross the blood-brain barrier to bind to tau in the brain, making them useful for in vivo detection or treatment of AD and other tauopathies in the brain.

Sequences of antibody CDRs and variable domains, as well as full length tau protein 2N4R and portions thereof, are shown in Table 1 below. In various embodiments, antibodies and binding fragments disclosed herein comprise one or more of the CDR and/or variable domain sequences disclosed in the table, and/or bind to one or more portions of tau protein 2N4R disclosed in the table.

TABLE 1

| SEQ | | |
|---|---|---|
| ID | | |
| NO | Polypeptide | Sequence |
| 1 | HCDR1 | GFTFSSFG |
| | (E7) | |
| 2 | HCDR2 | ISGDSNTI |
| | (E7) | |
| 3 | HCDR3 | ARTLAY |
| | (E7) | |
| 4 | LCDR1 (E7) | EDIYNR |
| 5 | LCDR2 (E7) | GAS |
| 6 | LCDR3 (E7) | LQYWSIPWT |
| 7 | H variable | DVQLVESGGGLVQPGGSRKLSCAASGFTFSSFGMHWVRQAPEK |
| | (E7) | GLEWVAYISGDSNTIYYADTVKGRFTISRDNPKNTLFLQMTSLRS |
| | | EDTAIYYCARTLAYWGQGTLVTVS |
| 8 | L variable | DIQMTQSSSSFSVSLGDRVTITCKASEDIYNRLAWYQQTPGNAPS |
| | (E7) | LLISGASGLETGVPSRFSGSGSGRDYTLSITSLQTEDVATYYCLQ |
| | | YWSIPWTFGGGTKLEIKR |
| 9 | tau 2N4R | MAEPRQEFEV MEDHAGTYGL GDRKDQGGYT |
| | | MHQDQEGDTD AGLKESPLQT PTEDGSEEPG |
| | | SETSDAKSTP TAEDVTAPLV DEGAPGKQAA |
| | | AQPHTEIPEG TTAEEAGIGD TPSLEDEAAG |
| | | HVTQARMVSK SKDGTGSDDK KAKGADGKTK |
| | | IATPRGAAPP GQKGQANATR IPAKTPPAPK |
| | | TPPSSGEPPK SGDRSGYSSP GSPGTPGSRS |
| | | RTPSLPTPPT REPKKVAVVR TPPKSPSSAK |
| | | SRLQTAPVPM PDLKNVKSKI GSTENLKHQP GGGKVQIINK |
| | | KLDLSNVQSK CGSKDNIKHV PGGGSVQIVY |
| | | KPVDLSKVTS KCGSLGNIHH KPGGGQVEVK |
| | | SEKLDFKDRV QSKIGSLDNI THVPGGGNKK IETHKLTFRE |
| | | NAKAKTDHGA EIVYKSPVVS GDTSPRHLSN |
| | | VSSTGSIDMV DSPQLATLAD EVSASLAKQG L |
| 10 | tau 188-227 | PPKSGDRSGYSSPGSPGTPGSRSRTPSLPTPPTREPKKVA |
| 11 | tau 210-221 | SRTPSLPTPPTR |
| 12 | tau 210-221 | SRTPSLPpTPPTR |
| | (pT217) | |
| 13 | tau 151-188 | IATPRGAAPP GQKGQANATR IPAKTPPAPK TPPSSGEP |
| 14 | tau 163-172 | KGQANATRIP |
| 15 | HCDR1 | GFNIKDYY |
| | (E2) | |
| 16 | HCDR2 | LDPENDHT |
| | (E2) | |
| 17 | HCDR3 | SYYKYDDY |
| | (E2) | |
| 18 | LCDR1 (E2) | QSLLDSDGKTY |
| 19 | LCDR2 (E2) | LVS |
| 20 | LCDR3 (E2) | WQGTHFPLT |
| 21 | H variable | EVQLQQSGAELVRPGALVKLSCKASGFNIKDYYMHWVRQRPDQ |
| | (E2) | GLEWIGWLDPENDHTIYDPRFQDRANLTADTSSNTAYLQLSSLTS |
| | | EDTAVYYCSYYKYDDYWGQGTTLTVS |
| 22 | L variable | DVVMTQTPLTLSVTIGQPASISCKSSQSLLDSDGKTYLNWLVQRP |
| | (E2) | GQSPKRLIYLVSKLDSGVPDRFTGSGSGTDFTLKINRVEAEDLGV |
| | | YYCWQGTHFPLTFGAGTKLELKR |
| 23 | HCDR1 | GFTLSSFG |
| | (149) | |
| 24 | HCDR2 | ISSGSSTI |
| | (149) | |
| 25 | HCDR3 | SRRLDY |
| | (149) | |
| 26 | LCDR1 | KSLLYKDGKTY |
| | (149) | |
| 27 | LCDR2 | LMS |
| | (149) | |
| 28 | LCDR3 | QQFVEYPLT |
| | (149) | |
| 29 | H variable | DVQLVESGGGLVQPGGSRKLSCAASGFTLSSFGMHWVRQAPEK |
| | (149) | GLEWVAYISSGSSTIYYADTVKGRFTISRDNPKNILFLQMTSLRSE |
| | | DTAIYYCSRRLDYWGQGTSVTVS |
| 30 | L variable | DIVITQDELSNPVTSGESVSISCRSSKSLLYKDGKTYLNWFLQRPG |
| | (149) | QSPQLLIYLMSTRASGVSDRFSGSGSGTDFTLEISRVKAEDVGVY |
| | | YCQQFVEYPLTFGTGTKLELQR |
| 31 | tau 210-221 | SRpTPSLPpTPPTR |
| | (pT212, | |
| | pT217) | |
| 32 | HCDR1 | GFTFSGFG |
| | (E7cl07) | |
| 33 | HCDR1 | GFTFGSFG |
| | (E7cl18) | |
| 34 | HCDR2 | VSGDSNTI |
| | (E7cl06) | |
| 35 | HCDR2 | ISGASSTI |
| | (E7cl13) | |
| 36 | HCDR2 | ISGDGNTI |
| | (E7cl17) | |
| 37 | HCDR2 | ISGDSNTV |
| | (E7cl18) | |
| 38 | HCDR2 | ISGDSSTI |
| | (E7cl29) | |
| 39 | LCDR1 | ENIYNR |
| | (E7cl21PD) | |
| 40 | LCDR2 | GAG |
| | (E7cl14) | |
| 41 | LCDR3 | LQYWSTPWT |
| | (E7cl03) | |
| 42 | LCDR3 | LQYRSIPWT |
| | (E7cl29) | |
| 43 | H variable | DAQLVESGGGLVQPGGSRKLSCAASGFTFSSFGMRWVRQAPEK |
| | (E7cl01) | GLEWVAYISGDSNTIYYADTVKGRFTISRDNPKNTLFLQMTSLRS |
| | | EDTAIYYCARTLAYWGQGTLVTVS |
| 44 | L variable | DIQMTQSSSSFSVSLGDRVTITCKASEDIYNRLAWYQQTPGNAPS |
| | (E7cl01) | LLISGASGLETGVPSRFSGSGSGRDYTLSITSLQTEDVATYYCLQ |
| | | YWSIPWTFGGGTKLEIKR |
| 45 | H variable | DVQLVESGGGLVQPGGSRKLSCAASGFTFSSFGMHWVRQAPEK |
| | (E7cl02) | GLEWVTYISGDSNTIYYADTVKGRFTISRDNPKNTMFLQMTSLRS |
| | | EDTAIYYCARTLAYWGQGTLVTVS |
| 46 | L variable | DIQMTQSSSSFSVSLGDRATITCKASEDIYNRLAWYQQTPGNAPS |
| | (E7cl02) | LLISGASGLETGVPSRFSGSGSGRDYTLSITSLQTEDVATYYCLQ |
| | | YWSIPWTFGGGTKLEIKR |
| 47 | H variable | DVQLVESGGGLVQPGGSRKLSCAASGFTFSSFGMHWVRQAPE |
| | (E7cl03) | MGLEWVAYISGDSNTIYYADTVEGRFTISRDNPKNTLSLQMTSLR |
| | | SEDTAIYYCARTLAYWGQGTLVTVS |
| 48 | L variable | DIQMTQSSSSFSVSLGDRVTITCKASEDIYNRLAWYQQTPGNAPS |
| | (E7cl03) | LLISGASGLEPGVPSRFSGSGSGRDYTLSITSLQTEDVATYYCLQ |
| | | YWSTPWTFGGGTKLEIKR |
| 49 | H variable | DVQLVESGGGLAQPGGSRKLSCAASGFTFSSFGMHWVRQAPEK |
| | (E7cl04) | GLEWVAYISGDSNTIYYADTEKGRFTISRDNPKNALFLQMTSLRS |
| | | EDTAIYYCARTLAYWGQGTLVTVS |
| 50 | L variable | DIQMTQSSSSFSVSLGDRVTVTCKASEDIYNRLAWYQQTPGNAP |
| | (E7cl04) | SLLISGASGLETGVPSRFSGSGSGRDYTLSITSLQTEDVATYYCL |
| | | QYWSIPWTFGGGTKLEIKR |
| 51 | H variable | DVQLVESGGGLVQPGGSRKLSCAASGFTFSSFGMHWVRQAPEK |
| | (E7cl05) | GLEWVAYISGDSNTIYYADTVKGRFTISRDNPKNTLFLQMTSLRE |
| | | DTAIYYCARTLAYWGQGALVTVS |
| 52 | L variable | DIQMTQSSSSFSVSLGDRVTITCKASEDIYNRLAWYQQTPGDAPS |
| | (E7cl05) | LLISGASGLETGVPSRFSGSGSGRDYTLSITSLQTEDVATYYCLQ |
| | | YWSIPWTFGGGTKLEIKR |
| 53 | H variable | DVQLVESGGGLVQPGGSRRLSCAASGFTFSSFGMHWVRQAPEK |
| | (E7cl06) | GLEWVAYVSGDSNTIYYADTVKDRFTISRDNPKNTLFLQMASLRS |
| | | EDTAIYYCARTLAYWGQGTLVTVS |
| 54 | L variable | DIQMTQSSSSFSVSLGDRVTITCKASEDIYNRLAWYQQTPGNAPS |
| | (E7cl06) | LLISGASGLETGVPSRFSGSGSGRDYTLSITSLQTEDVATYYCLQ |
| | | YWSIPWTFSGGTKLEIKR |
| 55 | H variable | DVQLVESGGGLVQPGGSRKLSCAASGFTFSGFGMHWVRQAPE |
| | (E7cl07) | KGLEWVAYISGDSNTIYYADTVKGRFTISRDNPKNTLSLQMTSLR |
| | | SEDTAIYYCARTLAYWGQGTLVTVS |
| 56 | L variable | DIQMTQSSSSFSVSLGDRVTITCKASEDIYNRLAWYQQTPGNAPS |
| | (E7cl07) | LLISGASGLETGVPSRFSGSGSGRDYTLSITSLQTEDVATYYCLQ |
| | | YWSIPWTFGGGTKLEIKR |
| 57 | H variable | DVQLVESGGGLVQPGGSRKLSCAASGFTFSSFGMHWVRQAPEK |
| | (E7cl07PD) | GLEWVAYISGDSNTIYYADTVKGRLTISRDNPKNTLFLQMTSLRSE |
| | | DTAIYYCARTLAYWGQGTLVTVS |
| 58 | L variable | DIQMTQSSSSFSVSLGDRVTITCKASEDIYNRLAWYQQIPGNAPS |
| | (E7cl07PD) | LLISGASGLETGVPSRFSGSGSGRDYTLSITSLQTEDVATYYCLQ |
| | | YWSIPWTFGGGTKLEIKR |
| 59 | H variable | DVQLVESGGGLVQPGGSRKLSCAASGFTFSSFGMHWVRQAPEK |
| | (E7cl08) | GLEWVAYISGDSNTIYYADTVKGRFTISRNNPKNTLFLQMTSLRS |
| | | EDTAIYYCARTLAYWGQGTLVTVS |
| 60 | L variable | DIQMTQSSSSLSVSLGDRVTITCKASEDIYNRLAWYQQTPGNAPS |
| | (E7cl08) | LLISGASGLETGVPSRFSGSGSGRDYTLSITSLQTEDVATYYCLQ |
| | | YWSIPWTFGGGTKLEIKR |
| 61 | H variable | DVQLVESGGGLVQPGGSRKLSCAASGFTFSSFGMHWARQAPEK |
| | (E7cl10) | GLEWVAYISGDSNTIYYADTVKGRFTISRDNPKNTLFLQMTSLRS |
| | | EDTAIYYCARTLAYWGQGTLVTVS |
| 62 | L variable | DIQMTQSSSSFSVSLGDRVTITCKASEDIYNRLAWYQQTPGSAPS |
| | (E7cl10) | LLISGASGLETGVPSRFSGSGSGRDYTLSITSLQTEDVATYYCLQ |
| | | YWSIPWTFGGGTKLEIKR |
| 63 | H variable | DVQLVESGGGLVQPGGSRKLSCAASGFTFSSFGMHWVRQAPEK |
| | (E7cl11) | GLEWVAYISGDSNTIYYADTVKGRFTISRDNPKNTLLLQMTSLRSE |
| | | DTAIYYCARTLAYWGQGTLVTVS |
| 64 | L variable | DIQMTQSSSSLSVSLGDRVTITCKASEDIYNRLAWYQQTPGNAPS |
| | (E7cl11) | LLISGASGLETGVPSRFSGSGSGRDYTLSITSLQTEDVATYYCLQ |
| | | YWSIPWTFGGGTKLGIKR |
| 65 | H variable | DVQLVESGGGLVQPGGSRKLSCAASGFTFSSFGMHWVRQAPEK |
| | (E7cl12) | GLEWIAYISGDSNTIYYADTVKGRFTISRDNPKNTLFLQMTSLRSE |
| | | DTAIYYCARTLAYWGQGTLVTVS |
| 66 | L variable | DIQMTQSSSSFSVSLGDRVTITCKASEDIYNRLAWYQQTPGNAPS |
| | (E7cl12) | LLISGASGLETGVPSRFSGSGSGRDYTLSITSLQTEDVATYYCLQ |
| | | YWSIPWTFGGGTKLEIKR |
| 67 | H variable | DVQLVESGGGLVQPGGSRKLSCAASGFTFSSFGMHWVRQAPEK |
| | (E7cl13) | GLEWVAYISGASSTIYYADTVKGRFTISRDNPKNTLFLQMTSLRSE |
| | | DTAIYYCARTLAYWGQGTLVTVS |
| 68 | L variable | DIQMTQSSSSFSVSLGDRVTITCKASEDIYNRLAWYQQTPGNAPS |
| | (E7cl13) | LLISGASGLETGVPSRFSGSGSGRDYTLSITSLQTEDVATYYCLQ |
| | | YWSIPWTFGGGTKLEIKR |
| 69 | H variable | DVQLVESGGGLVQPGGSRKLSCAASGFTFSSFGMHWARQAPEK |
| | (E7cl14) | GLEWVAYISGDSNTIYYADTVKGRLTISRDNPKNTLFLQTTSLRSE |
| | | DTAICYCARTLAYWGQGTLVTVS |
| 70 | L variable | DIQMTQSSSSFSVSLGDRVTITCKTSEDIYNRLAWYQQTPGNAPS |
| | (E7cl14) | LLIPGAGGLETGVPSRFSGSGSGRDYTLSITSLQTEDVATYYCLQ |
| | | YWSIPWTFGGGTKLEIKR |
| 71 | H variable | DVQLVESGGGLVQPGGSRRLSCAASGFTFSSFGMHWVRQAPG |
| | (E7cl15) | KGLEWVAYISGDSNTIYYADTVKGRFTISRDNPKNTLFLQMTSLR |
| | | SEDTAIYYCARTLAYWGQGTLVTVP |
| 72 | L variable | DIQMTQSSSSFSVSLGDRVTITCKASEDIYNRLAWYQQTPGNAPS |
| | (E7cl15) | LLISGASGLETGVPSRFSGSGSGRDYTLSITSLQTEDVATYYCLQ |
| | | YWSIPWTFGGGTKLEIKR |
| 73 | H variable | DVQLVESGGGLVQPGGSRKLSCAASGFTFSSFGMHWVRQAPEK |
| | (E7cl16) | GLEWVAYISGDSNTIYYADTVKGRFTISRDNPKNTLLLQMTSLRSE |
| | | DTAIYYCARTLAYWGQGTLVTVS |
| 74 | L variable | DIRMTQSSSSFSVSLGVRVTITCKASEDIYNRLAWYQQTPGNAPS |
| | (E7cl16) | LLISGASGLETGVPSRFSGSGSGRDYTLSITSLQTEDVATYYCLQ |
| | | YWSIPWTFGGGTKLEIKR |
| 75 | H variable | DVQLVESGGGLVQPGGSRRLSCAASGFTFSSFGMHWARQAPEK |
| | (E7cl17) | GLEWVAYISGDGNTIYYADTVKGRFTTSRDNPKNTLFLQMTSLRS |
| | | EDTAIYYCARTLAYWGQGTLVTVS |
| 76 | L variable | DIQMTQSSSSLSVSLGDRVTITCKASEDIYNRLAWYQQTPGSAPS |
| | (E7cl17) | LLISGASGLETGVPSRFSGSGSGRDYTLSITSLQTEDVATYYCLQ |
| | | YWSIPWTFGGGTKLEIKR |
| 77 | H variable | DVQLVESGGGLVQPGGSRKLSCAASGFTFGSFGMHWVRQAPE |
| | (E7cl18) | KGLEWVAYISGDSNTVYYADTVKGRFTISRDNPKNTLFLQMTSLR |
| | | SEDTAIYYCARTLAYWGQGTLVTVS |
| 78 | L variable | DIQMTQPSSSFSVSLGDRVTITCKASEDIYNRLAWYQQTPGNAPS |
| | (E7cl18) | LLISGASGLETGVPSRFSGSGSGGDYTLSITSLQTEDVATYYCLQ |
| | | YWSIPWTFGGGTKLEIKR |
| 79 | H variable | DVQLVESGGGLVQPGGSRKLSCAASGFTFSSFGMHWVRQAPEK |
| | (E7cl19) | GLEWVAYISGDSNTIYYADTVKGRFTISRDNPKNTLFLQMTSLRS |
| | | EDTAIYYCARTLAYWGQGTLVTVS |
| 80 | L variable | DIQMTQSSSSSSVSLGDRVTITCKASEDIYNRLAWYQQTPGNAPS |
| | (E7cl19) | LLISGASGLETGVPSRFSGSGSGRDYTLSITSLQTEDVATYYCLQ |
| | | YWSIPWTFGGGTKLEIKR |
| 81 | H variable | DVQLVESGGGLVQPGGSRKLSCAASGFTFSSFGMHWVRQAPEK |
| | (E7cl20) | GLEWVAYISGDSNTIYYADTAKGRFAISRDNPKNTLFLQMTSLRS |
| | | EDTAIYYCARTLAYWGQGTLVTVS |
| 82 | L variable | DIQMTQSSSSFSVSLGDRVTITCKASEDIYNRLAWYQQTPGSAPS |
| | (E7cl20) | LLISGASGLETGVPSRFSGSGSGRDYTLSITSLQTEDVATYYCLQ |
| | | YWSIPWTFGGGTKLEIKR |
| 83 | H variable | DVQLVESGGGLVQPGGSRKLSCAASGFTFSSFGMHWVRQAPEK |
| | (E7cl21PD) | GLEWVAYISGDSNTIYYADTVKGRFTISRDNPKSTLFLQMTSLRSE |
| | | DTAIYYCARTLAYWGQGTLVTVS |
| 84 | L variable | DIQMTQSSSSLSVSLGDRVAITCKASENIYNRLAWYQQTPGNAPS |
| | (E7cl21PD) | LLISGASGLETGVPSRFSGSGSGRDYTLSITSLQTEDVATYYCLQ |
| | | YWSIPWTFGGGTKLEIKR |
| 85 | H variable | DVQLVESGGGLVQPGGSRKLSCAASGFTFSSFGMHWARQAPEK |
| | (E7cl22) | GLEWVAYISGDSNTIYYADAVKGRFTISRDNPKNTLSLQMTSLRS |
| | | EDTAIYYCARTLAYWGQGTLVTVS |
| 86 | L variable | DIQMTQSSSSLSVSLGDRVTITCKASEDIYNRLAWYQQTPGNAPS |
| | (E7cl22) | LLISGASGLETGVPSRFSGSGSGRDYTLSITSLQTEDVATYYCLQ |
| | | YWSIPWTFGGGTKLEIKR |
| 87 | H variable | DVQLVESGRGLVQPGGSRKLSCAASGFTFSSFGMHWVRQAPEK |
| | (E7cl23) | GLEWVAYISGDSNTIYYAGTVKGRFTISRDNPKNTLFLQMTSLRS |
| | | EDTAIYYCARTLAYWGQGTLVTVS |
| 88 | L variable | DIQMTQSSSSSSVPLGDRVTITCKASEDIYNRLAWYQQTPGNAPS |
| | (E7cl23) | LLISGASGLETGVPSRFSGSGSGRDYTLSITSLQTEDVATYYCLQ |
| | | YWSIPWTFGGGTKLEIKR |
| 89 | H variable | DVQLVESGGGLVQPGGSRKLSCAASGFTFSSFGMHWARQAPEK |
| | (E7cl26) | GLEWVAYISGDSNTIYYADTVKGRFTISRDNPKNTLFLQMTSLRS |
| | | EDTAIYYCARTLAYWGQGTLVTVS |
| 90 | L variable | DIQMTQSSSSFSVSLGDRVTITCKASEDIYNRLAWYQQTPGNAPS |
| | (E7cl26) | LLISGASGLETGVPSRFSGSGSGRDYTLSVTSLQTEDVATYYCLQ |
| | | YWSIPWTFGGGTKLEIKR |
| 91 | H variable | GVRLVESGGGLVQPGGSRKLSCAASGFTFSSFGMHWVRQAPEK |
| | (E7cl27) | GLEWVAYISGDSNTIYYADTVKGRFTISRDNPESTLFLQMTSLRSE |
| | | DTAIYYCARTLAYWGQGTLVTVS |
| 92 | L variable | DIQMTQSSSSFSVSLGDRVTITCKASEDIYNRLAWYQQTPGNAPS |
| | (E7cl27) | LLISGASGLETGVPSRFSGSGSGRDYTLSITSLQTEDVATYYCLQ |
| | | YWSIPWTFGGGTKLEIKR |
| 93 | H variable | DVQLVESGGGLVQPGGSRKLSCAASGFTFSSFGMHVVVRQAPEK |
| | (E7cl28) | GLEWVAYISGDSNTIYYADTVKGRFTISRDNPKNTLFLQMTSLRS |
| | | EDTAIYYCARTLAYWGQGTLVTVS |
| 94 | L variable | DIQMTQSSSSLSVSLGDRVTITCKASEDIYNRLAWYQQTPGNAPS |
| | (E7cl28) | LLISGASGLETGVPSRFSGSGSGRDYTLSITSLQTEDVATYYCLQ |
| | | YWSIPWTFGGGTKLEIKR |
| 95 | H variable | DVQLVESGGGLVQPGGSRKLSCAASGFTFSSFGMHWVRQAPEK |
| | (E7cl29) | GLEWVAYISGDSSTIYYADTVKGRFTISRDNPKNTLFLQMTSLRPE |
| | | DTAIYYGARTLAYWGQGTLVTVS |
| 96 | L variable | DIQMTQSSSSLSVSLGDRVTITCEASEDIYNRLAWYQQTPGDAPS |
| | (E7cl29) | LLISGASGLETGVPSRFSGSGSGRDHTLSITSLQTEDVATYYCLQ |
| | | YRSIPWTFGGGTKLEVKR |
| 97 | H variable | DVQLVESGGGLVQPGGSRKLSCAASGFTFSSFGMHWVRQAPEK |
| | (E7cl30) | GLEWVAYISGDSNTIYYADTVKGRFTISRDNPKNTLHLQMTSLRS |
| | | EDTAIYYCARTLAYWGQGTPVTVS |
| 98 | L variable | DIQMTQSSSSFSVSLGDRVTITCKASEDIYNRLAWYQQTPGNAPS |
| | (E7cl30) | LLISGASGLETGVPSRFSGSGSGRDYTLSITSLQTEDVATYYCLQ |
| | | YWSIPWTFGGGTKLEIKR |
| 99 | H variable | DVQLVESGGGLVQPGGSRKLSCAASGFTLSSFGMHWVRQAPEK |
| | (DC149) | GLEWVAYISSGSSTIYYADTVKGRFTISRDNPKNILFLQMTSLRSE |
| | | DTAIYYCSRRLDYWGQGTSVTVS |
| 100 | L variable | DIVITQDELSNPVTSGESVSISCRSSKSLLYKDGKTYLNWFLQRPG |
| | (DC149) | QSPQLLIYLMSTRASGVSDRFSGSGSGTDFTLEISRVKAEDVGVY |
| | | YCQQFVEYPLTFGTGTKLELQR |
| 101 | HCDR1 | GFTFSNYA |
| | (DC807) | |
| 102 | HCDR2 | ISSGGTI |
| | (DC807) | |
| 103 | HCDR3 | ARVNYDYGYAMDY |
| | (DC807) | |
| 104 | LCDR1 | QSLENSNGNTY |
| | (DC807) | |
| 105 | LCDR2 | RVS |
| | (DC807) | |
| 106 | LCDR3 | LQVTHVPWT |
| | (DC807) | |
| 107 | H variable | EVKLVESGGGLVKPGGSLKLSCAASGFTFSNYAMSWVRQNPEK |
| | (DC807) | RLEWVASISSGGTIYSPDSVKGRFTISRDNGRNILYLQMSSLRSE |
| | | DTAMYSCARVNYDYGYAMDYWGQGTSVTVS |
| 108 | L variable | DAVMTQTPLSLPVSLGDQASISCRSSQSLENSNGNTYLNWYLQK |
| | (DC807) | PGQSPQLLIYRVSNRFSGVLDRFSGSGSGTDFTLKISRVEAEDLG |
| | | VYFCLQVTHVPWTFGGGTKLEIKR |

In various embodiments, antibodies or antigen binding fragments thereof capable of binding tau disclosed herein comprise one or more of the amino acid sequences disclosed in Table 1 above. In some embodiments, antibodies disclosed herein comprise variants of one or more sequences disclosed in Table 1 while retaining the ability to bind tau, e.g., tau phosphorylated at position 217.

For example, in some embodiments an antibody or antigen binding fragment thereof capable of binding tau comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, or SEQ ID NO: 1 with a substitution at one or more of position 5 and 6, HCDR2 comprises the amino acid sequence of SEQ ID NO: 2, or SEQ ID NO: 2 with a substitution at one or more of position 1, 4, 5, 6, and 8, HCDR3 comprises the amino acid sequence of SEQ ID NO: 3, and/or wherein LCDR1 comprises the amino acid sequence of SEQ ID NO: 4 or SEQ ID NO: 4 with a substitution at position 2, LCDR2 comprises the amino acid sequence of SEQ ID NO: 5, or SEQ ID NO: 5 with a substitution at position 3 and LCDR3 comprises the amino acid sequence of SEQ ID NO: 6, or SEQ ID NO: 6 with a substitution at one or more of position 4 and 6.

A substitution at a particular position may be determined by counting from left to right (amino to carboxy terminal) in an amino acid sequence, e.g., a sequence listed in Table 1 beginning at the amino terminal end of the peptide sequence (or the left end in the table).

In various embodiments, the substitution at position 5 in HCDR1 is glycine, the substitution at position 6 in HCDR1 is glycine. In some embodiments, the substitution at position 1 in HCDR2 is valine, the substitution at position 4 in HCDR2 is alanine, the substitution at position 5 in HCDR2 is glycine, the substitution at position 6 in HCDR2 is serine, and/or the substitution at position 8 in HCDR2 is valine. In some embodiments, the substitution at position 2 in LCDR1 is asparagine. In some embodiments, the substitution at position 3 in LCDR2 is glycine. In some embodiments, the substitution at position 4 of LCDR3 is arginine, and/or the substitution at position 6 in LCDR3 is threonine.

In various embodiments, HCDR1 comprises the amino acid sequence of SEQ ID NO: 1 with a substitution at position 5, HCDR2 comprises the amino acid sequence of SEQ ID NO: 2 with a substitution at 8 position 8, and/or HCDR3 comprises the amino acid sequence of SEQ ID NO: 3, and/or wherein LCDR1 comprises the amino acid sequence of SEQ ID NO: 4 with a substitution at position 2, LCDR2 comprises the amino acid sequence of SEQ ID NO: 5, and/or LCDR3 comprises the amino acid sequence of SEQ ID NO: 6.

In various embodiments, the substitution at position 5 in HCDR1 is glycine, and the substitution at position 8 in HCDR2 is valine, and the substitution at position 2 in LCDR1 is asparagine.

In various embodiments, the antibody or antigen binding fragment that comprises variant sequences of those disclosed in Table 1 comprises any of the antibody CDRs (e.g., all six CDRs) and/or full heavy and/or light chain variable domain sequences (e.g., paired sets of heavy and light chain variable domain sequences of a particular antibody clone) selected from those shown in FIGS. 24-27B.

In various embodiments, an antibody or antigen binding fragment thereof capable of binding tau is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In varios embodiments, the CDRs and/or variable domain sequences are selected from those listed in Table 1. In some embodiments, the HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6.

In various embodiments, an antibody or antigen binding fragment thereof capable of binding tau is disclosed, wherein the the heavy chain variable region comprises the amino acid sequence of SEQ ID NO: 7 or SEQ ID NO: 7 with a substitution at one or more of position 1, 2, 3, 9, 12, 19, 30, 31, 35, 37, 42, 43, 48, 49, 51, 54, 55, 56, 58, 62, 63, 64, 65, 66, 68, 69, 70, 73, 76, 77, 78, 79, 80, 83, 84, 88, 94, 96, 107, 108, and 112, and/or the light chain variable region comprises the amino acid sequence of SEQ ID NO: 8 or SEQ ID NO: 8 with a substitution at one or more of position 3, 7, 11, 14, 17, 19, 20, 21, 24, 25, 28, 39, 42, 49, 52, 56, 69, 71, 75, 92, 94, 99, 105, and 106.

In some embodiments, the heavy chain variable region comprises the amino acid sequence of SEQ ID NO: 7 with a substitution at one or more of position 1, 2, 3, 9, 12, 19, 30, 31, 35, 37, 42, 43, 48, 49, 51, 54, 55, 56, 58, 62, 63, 64, 65, 66, 68, 69, 70, 73, 76, 77, 78, 79, 80, 83, 84, 88, 94, 96, 107, 108, and 112, wherein the substitution in the heavy chain variable region at position 1 is glycine, at position 2 is alanine, at position 3 is arginine, at position 9 is arginine, at position 12 is alanine, at position 19 is arginine, at position 30 is glycine, at position 31 is glycine, at position 35 is arginine, at position 37 is alanine, at position 42 is glycine, at position 43 is methionine, at position 48 is isoleucine, at position 49 is threonine, at position 51 is valine, at position 54 alanine, at position 55 is glycine, at position 56 is serine, at position 58 is valine, at position 62 is glycine, at position 63 is alanine, at position 64 is selected from alanine and glutamic acid, at position 65 is glutamic acid, at position 66 is aspartic acid, at position 68 is leucine, at position 69 is alanine, at position 70 is threonine, at position 73 is asparagine, at position 76 is glutamic acid, at position 77 is serine, at position 78 is alanine, at position 79 is methionine, at position 80 is selected from serine, leucine, and histidine, at position 83 is threonine, at position 84 is alanine, at position 88 is proline, at position 94 is cysteine, at position 95 is glycine, at position 107 is alanine, at position 108 is proline, and/or at position 112 is proline.

In some embodiments, the light chain variable region comprises the amino acid sequence of SEQ ID NO: 8 with a substitution at one or more of position 3, 7, 11, 14, 17, 19, 20, 21, 24, 25, 28, 39, 42, 49, 52, 56, 69, 71, 75, 92, 94, 99, 105, and 106, wherein the substitution in the light chain variable region at position 3 is arginine, at position 7 is proline, at position 11 is selected from serine or leucine, at position 14 is proline, at position 17 is valine, at position 19 is alanine, at position 20 is alanine, at position 21 is valine, at position 24 is glutamic acid, at position 25 is threonine, at position 28 asparagine, at position 39 isoleucine, at position 42 is selected from serine and aspartic acid, at position 49 is proline, at position 52 is glycine, at position 56 is proline, at position 69 is glycine, at position 71 is histidine, at position 75 is valine, at position 92 is arginine, at position 94 is threonine, at position 99 is serine, at position 105 is glycine, and/or at position 106 is valine.

In various embodiments, the heavy chain variable region comprises the amino acid sequence of SEQ ID NO: 7 with a substitution at one or more of position 1, 3, 30, 37, 48, 58, 63, 68, 76, 77, and 80, and/or the light chain variable region comprises the amino acid sequence of SEQ ID SEQ ID NO: 8 with a substitution at one or more of position 7, 11, 20, 28, 39, and 69.

In some embodiments, the substitution in the heavy chain variable region at position 2 is alanine and/or the substitution at position 35 is arginine.

In some embodiments, the substitution in the heavy chain variable region at position 49 is threonine and/or at position 79 methionine, and/or the substitution in the light chain variable region at position 19 is alanine.

In some embodiments, the substitution in the heavy chain variable region at position 43 is methionine, at position 65 is glutamic acid, and/or at position 80 is serine, and/or the substitution in the light chain variable region at position 56 is proline and at position 94 is threonine.

In some embodiments, the substitution in the heavy chain variable region at position 12 is alanine, at position 64 is glutamic acid, and at position 78 is alanine, and/or the substitution in the light chain variable region at position 21 is valine.

In some embodiments, the substitution in the heavy chain variable region at position 107 is alanine, and/or the substitution in the light chain variable region at position 42 is aspartic acid.

In some embodiments, the substitution in the heavy chain variable region at position 19 is arginine, at position 51 is valine, at position 66 is aspartic acid, and at position 84 is alanine, and/or the substitution in the light chain variable region at position 99 is serine.

In some embodiments, the substitution in the heavy chain variable region at position 31 is glycine and/or at position 80 is serine.

In some embodiments, the substitution in the heavy chain variable region at position 73 is asparagine, and/or the substitution in the light chain variable region at position 11 is leucine.

In some embodiments, the substitution in the heavy chain variable region at position 37 is alanine, and/or the substitution in the light chain variable region at position 42 is serine.

In some embodiments, the substitution in the heavy chain variable region at position 80 is leucine, and/or the substitution in the light chain variable region at position 11 is leucine and at position 105 is glycine.

In some embodiments, the substitution in the heavy chain variable region at position 54 is alanine and/or at position 56 is serine.

In some embodiments, the substitution in the heavy chain variable region at position 37 is alanine, at position 68 is leucine, at position 83 is threonine, at position 99 is cysteine, the substation in the light chain variable region at position 25 is threonine, at position 49 is proline, and/or at position 52 is glycine.

In some embodiments, the substitution at position in the heavy chain variable region at position 19 is arginine, at position 42 is glycine, and/or at position 112 is proline.

In some embodiments, the substitution in heavy chain variable region at position 80 is leucine, and the substitution in the light chain variable region at position 3 is arginine and/or at position 17 is valine.

In some embodiments, the substitution in the heavy chain variable region at position 19 is arginine, at position 37 is alanine, at position 55 is glycine, at position 70 is threonine, the substitution in the light chain variable region at position 11 is leucine and/or at position 42 is serine.

In some embodiments, the substitution in the heavy chain variable region at position 64 is alanine, the substitution at position 69 is alanine, and/or the substitution in the light chain variable region at position 42 is serine.

In some embodiments, the substitution in the heavy chain variable region at position 9 is arginine and/or at position 62 is glycine, and/or the substitution in the light chain variable region at position 11 is serine and/or at position 14 is proline.

In some embodiments, the substitution in the heavy chain variable region at position 37 is alanine, and/or the substitution in the light chain variable region at position 75 is valine.

In some embodiments, the substitution at position in the heavy chain variable region at position 56 is serine, at position 88 is proline, and/or at position 96 is glycine, and/or the substitution in the light chain variable region at position 11 is leucine, at position 24 is glutamic acid, at position 42 is aspartic acid, at position 71 is histidine, at position 92 is arginine, and/or at position 106 is valine.

In some embodiments, the the substitution in the heavy chain variable region at position 80 is histidine and/or at position 108 is proline.

In some embodiments, the antibody or antigen binding fragment thereof capable of binding tau, comprises a heavy chain variable region comprising the amino acid sequence of SEQ ID NO: 7 with a substitution at one or more of position 1, 3, 30, 37, 48, 58, 63, 68, 76, 77, and 80, and a light chain variable region comprises the amino acid sequence of SEQ ID SEQ ID NO: 8 with a substitution at one or more of position 7, 11, 20, 28, 39, and 69. In various embodiments, the substitution in the heavy chain variable region at position 1 is glycine, at position 3 is arginine, at position 30 is glycine, at position 37 is alanine, at position 48 is isoleucine, at position 58 is valine, at position 63 is alanine, at position 68 is leucine, at position 76 is glutamic acid, at position 77 is serine, and/or at position 80 is serine, and the substitution in the light chain variable region at position 7 is proline, at position 11 is selected from leucine and serine, at position 20 is alanine, at position 28 is asparagine, at position 39 is isoleucine, and/or at position 69 is glycine.

In various embodiments, the substitution in the heavy chain variable region at position 68 is leucine, and/or the substitution in the light chain variable region at position 39 is isoleucine.

In various embodiments, the substitution in the heavy chain variable region at position 48 is isoleucine.

In various embodiments, the substitution in the heavy chain variable region at position 30 is glycine and/or at position 58 is valine, and the substitution in the light chain variable region at position 7 is proline and/or at position 69 is glycine.

In various embodiments, the substitution in the light chain variable region at position 11 is serine.

In various embodiments, the substitution in the heavy chain variable region at position 77 is serine, and/or the substitution in the light chain variable region at position 11 is leucine, at position 20 is alanine, and/or at position 28 is asparagine.

In various embodiments, the substitution in the heavy chain variable region at position 37 is alanine, at position 63 is alanine, and/or position 80 is serine, and/or the substitution in the light chain variable region at position 11 is leucine.

In various embodiments, the substitution in the heavy chain variable region at position 1 is glycine, at position 3 is arginine, at position 76 is glutamic acid, and/or at position 77 is serine.

In various embodiments, the substitution in the light chain variable region at position 11 is leucine.

In some embodiments, the heavy chain variable region comprises an amino acid sequence of SEQ ID NO: 7 and the light chain variable region comprises an amino acid sequence of SEQ ID NO: 8.

In some embodiments, an antibody or antigen binding fragment thereof capable of binding tau, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 23, HCDR2 comprises the amino acid sequence of SEQ ID NO: 24, and HCDR3 comprises the amino acid sequence of SEQ ID NO: 25; and wherein LCDR1 comprises the amino acid sequence of SEQ ID NO: 26, LCDR2 comprises the amino acid sequence of SEQ ID NO: 27, and LCDR3 comprises the amino acid sequence of SEQ ID NO: 28. In some embodiments, the heavy chain variable region comprises an amino acid sequence of SEQ ID NO: 29 and the light chain variable region comprises an amino acid sequence of SEQ ID NO: 30.

In various embodiments, an antibody or antigen binding fragment thereof capable of binding tau is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, or SEQ ID NO: 1 with a substitution at one or more of position 5 and 6, HCDR2 comprises the amino acid sequence of SEQ ID NO: 2, or SEQ ID NO: 2 with a substitution at one or more of position 1, 4, 5, 6, and 8, HCDR3 comprises the amino acid sequence of SEQ ID NO: 3, LCDR1 comprises the amino acid sequence of SEQ ID NO: 4, or SEQ ID NO: 4 with a substitution at position 2, LCDR2 comprises the amino acid sequence of SEQ ID NO: 5, or SEQ ID NO: 5 with a substitution at position 3, and LCDR3 comprises the amino acid sequence of SEQ ID NO: 6, or SEQ ID NO: 6 with a substitution at one or more of position 4 and 6. In various embodiments, the substitution at position 5 in HCDR1 is glycine, the substitution at position 6 in HCDR1 is glycine, the substitution at position 1 in HCDR2 is valine, the substitution at position 4 in HCDR2 is alanine, the substitution at position 5 in HCDR2 is glycine, the substitution at position 6 in HCDR2 is serine, the substitution at position 8 in HCDR2 is valine, the substitution at position 2 in LCDR1 is asparagine, the substitution at position 3 in LCDR2 is glycine, and/or the substitution at position 4 of LCDR3 is arginine, and/or the substitution at position 6 in LCDR3 is threonine.

In various embodiments, an antibody or antigen binding fragment thereof capable of binding tau wherein HCDR1 comprises the amino acid sequence of SEQ ID NO: 1 with a substitution at position 5, HCDR2 comprises the amino acid sequence of SEQ ID NO: 2 with a substitution at position 8, HCDR3 comprises the amino acid sequence of SEQ ID NO: 3, LCDR1 comprises the amino acid sequence of SEQ ID NO: 4 with a substitution at position 2, LCDR2 comprises the amino acid sequence of SEQ ID NO: 5, and/or LCDR3 comprises the amino acid sequence of SEQ ID NO: 6.

In various embodiments, an antibody or antigen binding fragment thereof capable of binding tau wherein the substitution at position 5 in HCDR1 is glycine, the substitution at position 8 in HCDR2 is valine, and/or the substitution at position 2 in LCDR1 is asparagine.

In various embodiment, the antibody or antigen binding fragment thereof capable of binding tau comprises an HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 41; or

an HCDR1 comprises the amino acid sequence of SEQ ID NO: 1, HCDR2 comprises the amino acid sequence of SEQ ID NO 34, and HCDR3 comprises the amino acid sequence of SEQ ID NO: 3; and wherein LCDR1 comprises the amino acid sequence of SEQ ID NO: 4, LCDR2 comprise the amino acid sequence of SEQ ID NO: 5, and LCDR3 comprise the amino acid sequence of SEQ ID NO 6; or

an HCDR1 comprises the amino acid sequence of SEQ ID NO: 32, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or

an HCDR1 comprises the amino acid sequence of SEQ ID NO: 1, HCDR2 comprises the amino acid sequence of SEQ ID NO: 35, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or an HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 40, and LCDR3 comprises the amino acid sequence of SEQ ID NO: 6; or

an HCDR1 comprises the amino acid sequence of SEQ ID NO: 1, HCDR2 comprises the amino acid sequence of SEQ ID NO: 36, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or

an HCDR1 comprises the amino acid sequence of SEQ ID NO: 33, HCDR2 comprises the amino acid sequence of SEQ ID NO: 37, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or

an HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 39, LCDR2 comprises the amino acid sequence of SEQ ID NO: 5, and LCDR3 comprises the amino acid sequence of SEQ ID NO: 6; or

an HCDR1 comprises the amino acid sequence of SEQ ID NO: 1, HCDR2 comprises the amino acid sequence of SEQ ID NO: 38, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 42; or

an HCDR1 comprises the amino acid sequence of SEQ ID NO: 33, HCDR2 comprises the amino acid sequence of SEQ ID NO: 37, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or

an HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 39, LCDR2 comprises the amino acid sequence of SEQ ID NO: 5, and LCDR3 comprises the amino acid sequence of SEQ ID NO: 6.

In various embodiments, an antibody or antigen binding fragment thereof capable of binding tau comprising a heavy chain variable region and a light chain variable region, wherein the heavy chain variable region comprises the amino acid sequence of SEQ ID NO: 43 and the light chain variable region comprises the amino acid sequence of SEQ ID NO: 44; or the heavy chain variable region comprises the amino acid sequence of SEQ ID NO: 45 and the light chain variable region comprises the amino acid sequence of SEQ ID NO: 46; or the heavy chain variable region comprises the amino acid sequence of SEQ ID NO: 47 and the light chain variable region comprises the amino acid sequence of SEQ ID NO: 48; or the heavy chain variable region comprises the amino acid sequence of SEQ ID NO: 49 and the light chain variable region comprises the amino acid sequence of SEQ ID NO: 50; or the heavy chain variable region comprises the amino acid sequence of SEQ ID NO: 51 and the light chain variable region comprises the amino acid sequence of SEQ ID NO: 52; or

the heavy chain variable region comprises the amino acid sequence of SEQ ID NO: 53 and the light chain variable region comprises the amino acid sequence of SEQ ID NO: 54; or the heavy chain variable region comprises the amino acid sequence of SEQ ID NO: 55 and the light chain variable region comprises the amino acid sequence of SEQ ID NO: 56; or the heavy chain variable region comprises the amino acid sequence of SEQ ID NO: 57 and the light chain variable region comprises the amino acid sequence of SEQ ID NO: 58; or the heavy chain variable region comprises the amino acid sequence of SEQ ID NO: 59 and the light chain variable region comprises the amino acid sequence of SEQ ID NO: 60; or the heavy chain variable region comprises the amino acid sequence of SEQ ID NO: 61 and the light chain variable region comprises the amino acid sequence of SEQ ID NO: 62; or

the heavy chain variable region comprises the amino acid sequence of SEQ ID NO: 63 and the light chain variable region comprises the amino acid sequence of SEQ ID NO: 64; or the heavy chain variable region comprises the amino acid sequence of SEQ ID NO: 65 and the light chain variable region comprises the amino acid sequence of SEQ ID NO: 66; or the heavy chain variable region comprises the amino acid sequence of SEQ ID NO: 67 and the light chain variable region comprises the amino acid sequence of SEQ ID NO: 68; or the heavy chain variable region comprises the amino acid sequence of SEQ ID NO: 69 and the light chain variable region comprises the amino acid sequence of SEQ ID NO: 70; or the heavy chain variable region comprises the amino acid sequence of SEQ ID NO: 71 and the light chain variable region comprises the amino acid sequence of SEQ ID NO: 72; or the heavy chain variable region comprises the amino acid sequence of SEQ ID NO: 73 and the light chain variable region comprises the amino acid sequence of SEQ ID NO: 74; or the heavy chain variable region comprises the amino acid sequence of SEQ ID NO: 75 and the light chain variable region comprises the amino acid sequence of SEQ ID NO: 76; or the heavy chain variable region comprises the amino acid sequence of SEQ ID NO: 77 and the light chain variable region comprises the amino acid sequence of SEQ ID NO: 78; or the heavy chain variable region comprises the amino acid sequence of SEQ ID NO: 79 and the light chain variable region comprises the amino acid sequence of SEQ ID NO: 80; or the heavy chain variable region comprises the amino acid sequence of SEQ ID NO: 81 and the light chain variable region comprises the amino acid sequence of SEQ ID NO: 82; or the heavy chain variable region comprises the amino acid sequence of SEQ ID NO: 83 and the light chain variable region comprises the amino acid sequence of SEQ ID NO: 84; or the heavy chain variable region comprises the amino acid sequence of SEQ ID NO: 85 and the light chain variable region comprises the amino acid sequence of SEQ ID NO: 86; or the heavy chain variable region comprises the amino acid sequence of SEQ ID NO: 87 and the light chain variable region comprises the amino acid sequence of SEQ ID NO: 88; or the heavy chain variable region comprises the amino acid sequence of SEQ ID NO: 89 and the light chain variable region comprises the amino acid sequence of SEQ ID NO: 90; or the heavy chain variable region comprises the amino acid sequence of SEQ ID NO: 91 and the light chain variable region comprises the amino acid sequence of SEQ ID NO: 92; or the heavy chain variable region comprises the amino acid sequence of SEQ ID NO: 93 and the light chain variable region comprises the amino acid sequence of SEQ ID NO: 94; or the heavy chain variable region comprises the amino acid sequence of SEQ ID NO: 95 and the light chain variable region comprises the amino acid sequence of SEQ ID NO: 96; or the heavy chain variable region comprises the amino acid sequence of SEQ ID NO: 97 and the light chain variable region comprises the amino acid sequence of SEQ ID NO: 98; or the heavy chain variable region comprises the amino acid sequence of SEQ ID NO: 57 and the light chain variable region comprises the amino acid sequence of SEQ ID NO: 58; or the heavy chain variable region comprises the amino acid sequence of SEQ ID NO: 65 and the light chain variable region comprises the amino acid sequence of SEQ ID NO: 66; or the heavy chain variable region comprises the amino acid sequence of SEQ ID NO: 77 and the light chain variable region comprises the amino acid sequence of SEQ ID NO: 78; or the heavy chain variable region comprises the amino acid sequence of SEQ ID NO: 79 and the light chain variable region comprises the amino acid sequence of SEQ ID NO: 80; or the heavy chain variable region comprises the amino acid sequence of SEQ ID NO: 83 and the light chain variable region comprises the amino acid sequence of SEQ ID NO: 84; or the heavy chain variable region comprises the amino acid sequence of SEQ ID NO: 85 and the light chain variable region comprises the amino acid sequence of SEQ ID NO: 86; or the heavy chain variable region comprises the amino acid sequence of SEQ ID NO: 91 and the light chain variable region comprises the amino acid sequence of SEQ ID NO: 92; or the heavy chain variable region comprises the amino acid sequence of SEQ ID NO: 93 and the light chain variable region comprises the amino acid sequence of SEQ ID NO: 94.

Also disclosed herein, in various embodiments, are antibody or antigen binding fragments that can compete for binding with an antibody or antigen binding fragment thereof defined by sequence, as disclosed in this paragraph. In another embodiment, an antibody or antigen binding fragment thereof disclosed herein can bind to the same epitope on tau protein 2N4R (SEQ ID NO: 9) as that of an antibody or antigen binding fragment thereof defined by sequence, as disclosed in this paragraph.

In various embodiments, an antibody or antigen binding fragment thereof that is disclosed herein may bind to an epitope on tau comprising one or more of residues 188-227 of tau protein 2N4R (SEQ ID NO: 10). In some embodiments, the epitope comprises one or more of residues 210-221 of tau protein 2N4R (SEQ ID NO: 11). In various embodiments, an antibody or antigen binding fragment thereof that is disclosed herein may bind to an epitope on tau comprising residues 188-227 of tau protein 2N4R (SEQ ID NO: 10). In some embodiments, the epitope comprises residues 210-221 of tau protein 2N4R (SEQ ID NO: 11). The epitope may contain at least one, or more than one, phosphorylated residue, which may include a phospho-threonine at position 217 of tau protein 2N4R (SEQ ID NO: 9). In some embodiments, the epitope also comprises a phosphorylated serine at position 210, threonine at position 212, serine at position 214, or threonine at position 220 of tau protein 2N4R, or any combination thereof. In certain embodiments, the epitope comprises or consists of SRTPSLPpTPPTR (sequence of SEQ ID NO: 12). In certain embodiments, the epitope comprises or consists of a region comprising more than one phosphorylated residue, e.g., SRpTPSLPpTPPTR (sequence of SEQ ID NO: 31). In some embodiments, the epitope is determined by any method known to the skilled artisan. In some embodiments, alanine scanning or deletion/mutagenesis studies are used to determine the epitope. In some embodiments, mass spectrometry is used to determine the epitope. In certain embodiments, the epitope is determined by a crystal structure of the antibody-tau complex, where the epitope is defined as any contact residue on tau within 5 angstroms of the antibody binding pocket. In some embodiments, the antibody or antigen binding fragment comprises a heavy chain variable region amino acid sequence of SEQ ID NO: 7 and a light chain variable region amino acid sequence of SEQ ID NO: 8. In some embodiments, the antibody or antigen binding fragment comprises a heavy chain variable region amino acid sequence of SEQ ID NO: 29 and a light chain variable region amino acid sequence of SEQ ID NO: 30. In some embodiments, the antibody or antigen binding fragment comprises a heavy chain variable region comprises the amino acid sequence of SEQ ID NO: 7 with a substitution at one or more of position 1, 3, 30, 37, 48, 58, 63, 68, 76, 77, and 80, and/or light chain variable region comprises the amino acid sequence of SEQ ID SEQ ID NO: 8 with a substitution at one or more of position 7, 11, 20, 28, 39, and 69. In some embodiments, the substitution in the heavy chain variable region at position 68 is leucine, and the substitution in the light chain variable region at position 39 is isoleucine. In various embodiments, the substitution in the heavy chain variable region at position 48 is isoleucine. In various embodiments, the substitution in the heavy chain variable region at position 30 is glycine and at position 58 is valine, and the substitution in the light chain variable region at position 7 is proline and at position 69 is glycine. In various embodiments, the substitution in the light chain variable region at position 11 is serine. In various embodiments, the substitution in the heavy chain variable region at position 77 is serine, and the substitution in the light chain variable region at position 11 is leucine, at position 20 is alanine, and at position 28 is asparagine. In various embodiments, the substitution in the heavy chain variable region at position 37 is alanine, at position 63 is alanine, and position 80 is serine, and the substitution in the light chain variable region at position 11 is leucine. In various embodiments, the substitution in the heavy chain variable region at position 1 is glycine, at position 3 is arginine, at position 76 is glutamic acid, and at position 77 is serine. In various embodiments, the substitution in the light chain variable region at position 11 is leucine.

In other aspects, disclosed herein are antibodies that can compete for binding to tau protein 2N4R (SEQ ID NO: 9) with an antibody comprising a heavy chain variable region and a light chain variable region, wherein the heavy chain variable region comprises an amino acid sequence of SEQ ID NO: 7 and the light chain variable region comprises an amino acid sequence of SEQ ID NO: 8. In some embodiments, the antibody or antigen binding fragment comprises a heavy chain variable region amino acid sequence of SEQ ID NO: 29 and a light chain variable region amino acid sequence of SEQ ID NO: 30. In some embodiments, the antibody or antigen binding fragment comprises a heavy chain variable region comprises the amino acid sequence of SEQ ID NO: 7 with a substitution at one or more of position 1, 3, 30, 37, 48, 58, 63, 68, 76, 77, and 80, and/or light chain variable region comprises the amino acid sequence of SEQ ID SEQ ID NO: 8 with a substitution at one or more of position 7, 11, 20, 28, 39, and 69. In some embodiments, the substitution in the heavy chain variable region at position 68 is leucine, and the substitution in the light chain variable region at position 39 is isoleucine. In various embodiments, the substitution in the heavy chain variable region at position 48 is isoleucine. In various embodiments, the substitution in the heavy chain variable region at position 30 is glycine and at position 58 is valine, and the substitution in the light chain variable region at position 7 is proline and at position 69 is glycine. In various embodiments, the substitution in the light chain variable region at position 11 is serine. In various embodiments, the substitution in the heavy chain variable region at position 77 is serine, and the substitution in the light chain variable region at position 11 is leucine, at position 20 is alanine, and at position 28 is asparagine. In various embodiments, the substitution in the heavy chain variable region at position 37 is alanine, at position 63 is alanine, and position 80 is serine, and the substitution in the light chain variable region at position 11 is leucine. In various embodiments, the substitution in the heavy chain variable region at position 1 is glycine, at position 3 is arginine, at position 76 is glutamic acid, and at position 77 is serine. In various embodiments, the substitution in the light chain variable region at position 11 is leucine.

In various embodiments, competition is measured by flow cytometry or fluorescence microscopy, ELISA, HTRF and/or SPR. In one embodiment, competition ELISA is used to measure competitive binding. In another embodiment, a BIACORE assay is used. In some embodiments, the antibody or antigen binding fragment with which the disclosed antibody competes comprises a heavy chain variable region amino acid sequence of SEQ ID NO: 7 and a light chain variable region amino acid sequence of SEQ ID NO: 8. In some embodiments, the antibody or antigen binding fragment with which the disclosed antibody competes comprises a heavy chain variable region amino acid sequence of SEQ ID NO: 29 and a light chain variable region amino acid sequence of SEQ ID NO: 30. In some embodiments, the antibody or antigen binding fragment with which the disclosed antibody competes comprises a heavy chain variable region comprises the amino acid sequence of SEQ ID NO: 7 with a substitution at one or more of position 1, 3, 30, 37, 48, 58, 63, 68, 76, 77, and 80, and/or light chain variable region comprises the amino acid sequence of SEQ ID SEQ ID NO: 8 with a substitution at one or more of position 7, 11, 20, 28, 39, and 69. In some embodiments, the antibody or antibody fragment with which the disclosed antibody competes, comprises a substitution in the heavy chain variable region at position 68 is leucine, and the substitution in the light chain variable region at position 39 is isoleucine. In various embodiments, the substitution in the heavy chain variable region at position 48 is isoleucine. In various embodiments, the substitution in the heavy chain variable region at position 30 is glycine and at position 58 is valine, and the substitution in the light chain variable region at position 7 is proline and at position 69 is glycine. In various embodiments, the substitution in the light chain variable region at position 11 is serine. In various embodiments, the substitution in the heavy chain variable region at position 77 is serine, and the substitution in the light chain variable region at position 11 is leucine, at position 20 is alanine, and at position 28 is asparagine. In various embodiments, the substitution in the heavy chain variable region at position 37 is alanine, at position 63 is alanine, and position 80 is serine, and the substitution in the light chain variable region at position 11 is leucine. In various embodiments, the substitution in the heavy chain variable region at position 1 is glycine, at position 3 is arginine, at position 76 is glutamic acid, and at position 77 is serine. In various embodiments, the substitution in the light chain variable region at position 11 is leucine. In some embodiments, the antibody or antigen binding fragment can bind to the same one or more amino acids on tau protein 2N4R (SEQ ID NO: 9) as an antibody comprising a heavy chain variable region and a light chain variable region, wherein the heavy chain variable region comprises an amino acid sequence of SEQ ID NO: 7 and the light chain variable region comprises an amino acid sequence of SEQ ID NO: 8.

In various embodiments, the antibody or antigen binding fragment with which the disclosed antibody competes can bind to an epitope on tau comprising one or more of residues 188-227 of tau protein 2N4R (SEQ ID NO: 10). In certain embodiments, the epitope comprises one or more of residues 210-221 of tau protein 2N4R (SEQ ID NO: 11). In other embodiments, the epitope comprises at least one phosphorylated residue. In another embodiment, the at least one phosphorylated residue comprises a phospho-threonine at position 217 of tau protein 2N4R (SEQ ID NO: 9), and optionally also comprise a phosphorylated serine at position 210, threonine at position 212, serine at position 214, or threonine at position 220 of tau protein 2N4R, or any combination thereof. In another embodiment, the epitope comprises or consists of SRTPSLPpTPPTR (sequence of SEQ ID NO: 12). In certain embodiments, the epitope comprises or consists of SRpTPSLPpTPPTR (sequence of SEQ ID NO: 31).

In various embodiments, an antibody or antigen binding fragment thereof that can bind to a region or fragment of tau protein 2N4R comprising or consisting of residues 188-227 of tau protein 2N4R (SEQ ID NO: 10), wherein the region or fragment is phosphorylated. In various embodiments, the region or fragment comprises or consists of residues 210-221 of tau protein 2N4R (SEQ ID NO: 11). In various embodiments, the region or fragment is phosphorylated at a position corresponding to residue 217 of tau protein 2N4R, and optionally also at one or more of positions corresponding to serine 210, threonine 212, serine 214, and threonine 220 of tau protein 2N4R (SEQ ID NO: 9). In various embodiments, the region or fragment of tau protein 2N4R comprises or consists of SRTPSLPpTPPTR (sequence of SEQ ID NO. 12). In certain embodiments, the epitope comprises or consists of SRpTPSLPpTPPTR (sequence of SEQ ID NO: 31).

In various embodiments, an antibody or antigen binding fragment disclosed herein can bind a phosphorylated epitope on tau, e.g., as measured by flow cytometry or fluorescence microscopy, ELISA, HTRF and/or SPR. In one embodiment, ELISA as used to measure or check for binding to phosphorylated and dephosphorylated tau. In another embodiment, a BIACORE assay is used.

In various embodiments, disclosed herein is an antibody or antigen binding fragment thereof that can bind to an epitope on tau comprising one or more of residues 151-188 of tau protein 2N4R (SEQ ID NO: 13). In some embodiments, the epitope on tau comprises one or more of residues 163-172 of tau protein 2N4R (SEQ ID NO: 14). In various embodiments, disclosed herein is an antibody or antigen binding fragment thereof that can bind to an epitope on tau comprising residues 151-188 of tau protein 2N4R (SEQ ID NO: 13). In some embodiments, the epitope on tau comprises residues 163-172 of tau protein 2N4R (SEQ ID NO: 14). In certain embodiments, the epitope comprises or consist of KGQANATRIP (sequence of SEQ ID NO. 14). In certain embodiments, one or more of the residues on the epitope are phosphorylated, e.g., a phosphorylated threonine at position 169 of tau protein 2N4R.

In various embodiments, disclosed herein is an antibody or antigen binding fragment thereof that can bind to a portion or fragment of tau comprising residues 151-188 of tau protein 2N4R (SEQ ID NO: 13). In some embodiments, the antibody or antigen binding fragment thereof that can bind to a portion or fragment of tau comprising residues 163-172 of tau protein 2N4R (SEQ ID NO: 14).

In some embodiments, the epitope of an antibody disclosed herein is determined by any method known to the skilled artisan. In some embodiments, alanine scanning or deletion/mutagenesis studies are used to determine the epitope. In some embodiments, mass spectrometry is used to determine the epitope. In certain embodiments, the epitope is determined by a crystal structure of the antibody-tau complex, where the epitope is defined as any contact residue on tau within 5 angstroms of the antibody binding pocket.

In various embodiments, an antibody or antigen binding fragment thereof capable of binding tau is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20. In certain embodiments, the heavy chain variable region comprises an amino acid sequence of SEQ ID NO: 21 and the light chain variable region comprises an amino acid sequence of SEQ ID NO: 22. In various embodiments, an antibody or antigen binding fragments thereof can compete for binding to one or more amino acids on tau protein 2N4R (SEQ ID NO: 9) with the previously described antibody or antigen binding fragment thereof. In another embodiment, an antibody or antigen binding fragment thereof can bind the same epitope on tau protein 2N4R (SEQ ID NO: 9) bound by the antibody or antigen binding fragments previously disclosed in this paragraph. In an embodiment, an antibody or antigen binding fragment thereof can bind the fragment or region of tau protein 2N4R (SEQ ID NO: 9) bound by the antibody or antigen binding fragments previously disclosed in this paragraph.

In various embodiments, any of the antibodies or antigen binding fragments described herein can comprise a heavy chain constant region and a light chain constant region. The heavy chain constant region may be an IgG, IgM, IgA, IgD, and IgE isotype, or a derivative or fragment thereof that retains at least one effector function of the intact heavy chain. The heavy chain constant region may be a human IgG isotype. The heavy chain constant region may be a human IgG1 or human IgG4 isotypes. The heavy chain constant region may be a human IgG1 isotype. The light chain constant region may be a human kappa light chain or lambda light chain or a derivative or fragment thereof that retains at least one effector function of the intact light chain. The light chain constant region may be a human kappa light chain.

In various embodiments, any of the disclosed antibodies or antigen binding fragments may be a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In another embodiment, any of the disclosed antibodies or antigen binding fragments comprises human or human-derived heavy and light chain variable regions, including human frameworks or human frameworks with one or more backmutations. See, e.g., the backmutation strategies outlined in U.S. Pat. No. 7,566,771, the disclosure of which is incorporated herein by reference. In various embodiments, any of the disclosed antibodies or antigen binding fragments may be a Fab, Fab′, F(ab′)2, Fd, scFv, (scFv)2, scFv-Fc, or Fv fragment.

In various embodiments, antibody DC2E7 or a functional antigen binding fragment thereof is disclosed, wherein DC2E7 is an antibody produced by a hybridoma deposited under American Type Culture Collection Patent Deposit No. PTA-124992. In various embodiments, antibody DC2E2 or a functional antigen binding fragment thereof is disclosed, wherein DC2E2 is an antibody produced by a hybridoma deposited under American Type Culture Collection Patent Deposit No. PTA-124991. Also disclosed are antibodies and antigen binding fragments that can bind to the same epitope on tau protein 2N4R (SEQ ID NO: 9) or can compete for binding to tau protein 2N4R (SEQ ID NO: 9) with either antibody DC2E7 or antibody DC2E2.

In various embodiments, any of the disclosed antibodies or antigen binding fragments thereof may be conjugated to a second agent. The second agent may be, e.g., at least one detectable agent, including but not limited to, an enzyme, a radioisotope, a fluorophore, a nuclear magnetic resonance marker, or a heavy metal. In one embodiment, the at least one detectable label is a radioisotope. In some embodiments, the antibody or antigen binding fragment conjugated to a detectable label (e.g., a radiolabel) comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In some embodiments, the heavy chain variable region comprises an amino acid sequence of SEQ ID NO: 7 and the light chain variable region comprises an amino acid sequence of SEQ ID NO: 8.

In some embodiments, the antibody or antigen binding fragment conjugated to a detectable label (e.g., a radiolabel) comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises amino acid sequence of SEQ ID NO: 23, HCDR2 comprises the amino acid sequence of SEQ ID NO: 24, and HCDR3 comprises the amino acid sequence of SEQ ID NO: 25; and wherein LCDR1 comprises the amino acid sequence of SEQ ID NO: 26, LCDR2 comprises the amino acid sequence of SEQ ID NO: 27, and LCDR3 comprises the amino acid sequence of SEQ ID NO: 28. In some embodiments, In some embodiments, the heavy chain variable region comprises an amino acid sequence of SEQ ID NO: 29 and the light chain variable region comprises an amino acid sequence of SEQ ID NO: 30. In some embodiments, the antibody or antigen binding fragment thereof conjugated to a detectable label comprises a heavy chain variable region and a light chain variable region, wherein the heavy chain variable region comprises three heavy chain complementarity determining regions (HCDR1, HCDR2, and HCDR3), In some embodiments, the antibody or antigen binding fragment conjugated to a detectable label (e.g., a radiolabel) comprises a heavy chain variable region and a light chain variable region, wherein the heavy chain variable region comprises the amino acid sequence of SEQ ID NO: 43 and the light chain variable region comprises the amino acid sequence of SEQ ID NO: 44; or the heavy chain variable region comprises the amino acid sequence of SEQ ID NO: 45 and the light chain variable region comprises the amino acid sequence of SEQ ID NO: 46; or the heavy chain variable region comprises the amino acid sequence of SEQ ID NO: 47 and the light chain variable region comprises the amino acid sequence of SEQ ID NO: 48; or the heavy chain variable region comprises the amino acid sequence of SEQ ID NO: 49 and the light chain variable region comprises the amino acid sequence of SEQ ID NO: 50; or the heavy chain variable region comprises the amino acid sequence of SEQ ID NO: 51 and the light chain variable region comprises the amino acid sequence of SEQ ID NO: 52; or the heavy chain variable region comprises the amino acid sequence of SEQ ID NO: 53 and the light chain variable region comprises the amino acid sequence of SEQ ID NO: 54; or the heavy chain variable region comprises the amino acid sequence of SEQ ID NO: 55 and the light chain variable region comprises the amino acid sequence of SEQ ID NO: 56; or the heavy chain variable region comprises the amino acid sequence of SEQ ID NO: 57 and the light chain variable region comprises the amino acid sequence of SEQ ID NO: 58; or the heavy chain variable region comprises the amino acid sequence of SEQ ID NO: 59 and the light chain variable region comprises the amino acid sequence of SEQ ID NO: 60; or the heavy chain variable region comprises the amino acid sequence of SEQ ID NO: 61 and the light chain variable region comprises the amino acid sequence of SEQ ID NO: 62; or the heavy chain variable region comprises the amino acid sequence of SEQ ID NO: 63 and the light chain variable region comprises the amino acid sequence of SEQ ID NO: 64; or the heavy chain variable region comprises the amino acid sequence of SEQ ID NO: 65 and the light chain variable region comprises the amino acid sequence of SEQ ID NO: 66; or the heavy chain variable region comprises the amino acid sequence of SEQ ID NO: 67 and the light chain variable region comprises the amino acid sequence of SEQ ID NO: 68; or the heavy chain variable region comprises the amino acid sequence of SEQ ID NO: 69 and the light chain variable region comprises the amino acid sequence of SEQ ID NO: 70; or the heavy chain variable region comprises the amino acid sequence of SEQ ID NO: 71 and the light chain variable region comprises the amino acid sequence of SEQ ID NO: 72; or the heavy chain variable region comprises the amino acid sequence of SEQ ID NO: 73 and the light chain variable region comprises the amino acid sequence of SEQ ID NO: 74; or the heavy chain variable region comprises the amino acid sequence of SEQ ID NO: 75 and the light chain variable region comprises the amino acid sequence of SEQ ID NO: 76; or the heavy chain variable region comprises the amino acid sequence of SEQ ID NO: 77 and the light chain variable region comprises the amino acid sequence of SEQ ID NO: 78; or the heavy chain variable region comprises the amino acid sequence of SEQ ID NO: 79 and the light chain variable region comprises the amino acid sequence of SEQ ID NO: 80; or the heavy chain variable region comprises the amino acid sequence of SEQ ID NO: 81 and the light chain variable region comprises the amino acid sequence of SEQ ID NO: 82; or the heavy chain variable region comprises the amino acid sequence of SEQ ID NO: 83 and the light chain variable region comprises the amino acid sequence of SEQ ID NO: 84; or the heavy chain variable region comprises the amino acid sequence of SEQ ID NO: 85 and the light chain variable region comprises the amino acid sequence of SEQ ID NO: 86; or the heavy chain variable region comprises the amino acid sequence of SEQ ID NO: 87 and the light chain variable region comprises the amino acid sequence of SEQ ID NO: 88; or the heavy chain variable region comprises the amino acid sequence of SEQ ID NO: 89 and the light chain variable region comprises the amino acid sequence of SEQ ID NO: 90; or the heavy chain variable region comprises the amino acid sequence of SEQ ID NO: 91 and the light chain variable region comprises the amino acid sequence of SEQ ID NO: 92; or the heavy chain variable region comprises the amino acid sequence of SEQ ID NO: 93 and the light chain variable region comprises the amino acid sequence of SEQ ID NO: 94; or the heavy chain variable region comprises the amino acid sequence of SEQ ID NO: 95 and the light chain variable region comprises the amino acid sequence of SEQ ID NO: 96; or the heavy chain variable region comprises the amino acid sequence of SEQ ID NO: 97 and the light chain variable region comprises the amino acid sequence of SEQ ID NO: 98; or the heavy chain variable region comprises the amino acid sequence of SEQ ID NO: 57 and the light chain variable region comprises the amino acid sequence of SEQ ID NO: 58; or the heavy chain variable region comprises the amino acid sequence of SEQ ID NO: 65 and the light chain variable region comprises the amino acid sequence of SEQ ID NO: 66; or the heavy chain variable region comprises the amino acid sequence of SEQ ID NO: 77 and the light chain variable region comprises the amino acid sequence of SEQ ID NO: 78; or the heavy chain variable region comprises the amino acid sequence of SEQ ID NO: 79 and the light chain variable region comprises the amino acid sequence of SEQ ID NO: 80; or the heavy chain variable region comprises the amino acid sequence of SEQ ID NO: 83 and the light chain variable region comprises the amino acid sequence of SEQ ID NO: 84; or the heavy chain variable region comprises the amino acid sequence of SEQ ID NO: 85 and the light chain variable region comprises the amino acid sequence of SEQ ID NO: 86; or the heavy chain variable region comprises the amino acid sequence of SEQ ID NO: 91 and the light chain variable region comprises the amino acid sequence of SEQ ID NO: 92; or the heavy chain variable region comprises the amino acid sequence of SEQ ID NO: 93 and the light chain variable region comprises the amino acid sequence of SEQ ID NO: 94.

In some embodiments, the antibody or antigen binding fragment conjugated to a detectable label (e.g., a radiolabel) may compete for binding or bind the same epitope as this antibody defined by sequence.

Examples of suitable types of detectable labels consistent with the present disclosure include, but are not limited to, enzymes, radioisotopes and radionuclides, colloidal metals, fluorescent compounds, chemiluminescent compounds, biotinyl groups, predetermined polypeptide epitopes recognized by a secondary reporter (e.g., leucine zipper pair sequences, binding sites for secondary antibodies, metal binding domains, epitope tags), and chemi/electrochemi/bioluminescent compounds. The enzymes include, but are not limited to, peroxidase (e.g., horse radish peroxidase), luciferase, alkaline phosphatase, β-galactosidase, glucose oxidase, glucose amylase, carbonic anhydrase, acetylcholinesterase, lysozyme, malate dehydrogenase, or glucose-6 phosphate dehydrogenase. Alternatively, the label may be biotin, digoxin-genin, or 5-bromo-desoxyuridine. Fluorescent labels can be also combined with the antibodies and antigen binding fragments thereof, including rhodamine, lanthanide phosphors, fluorescein and its derivatives, fluorochromes, rhodamine and its derivatives, green fluorescent protein (GFP), red fluorescent protein (RFP), dansyl, umbelliferone, and others. Such conjugates may be prepared by methods known to a person skilled in the art. They can be bound with labels directly; via a spacer group or linkage group such as polyaldehyde, glutaraldehyde, ethylenediaminetetraacetic acid (EDTA) or diethylenetriaminepentaacetic acid (DPTA); or in the presence of other binding agents such as those routinely known in the art.

Other detectable labels may include radioactive labels such as iodine-123, iodine-125, iodine-126, iodine-133, iodine-131, bromine-77, technetium-99m, indium-113m, gallium-67, gallium-68, ruthenium-95, ruthenium-97, ruthenium-103, ruthenium-106, mercury-203, scandium-47, tellurium-121m, tellurium-128, thulium-165, thulium-167, thulium-168, fluorine-18, yttrium-99, and zirconium-89. Existing methods known to a person skilled in the art for labelling antibodies with radioisotypes, either directly or indirectly, e.g., via a chelating agent such as EDTA or DTPA, can be used.

In various embodiments, any of the disclosed antibodies or antigen binding fragments can be conjugated to a second agent, where the second agent may be at least one therapeutic agent for Alzheimer's disease or another tauopathy. In some embodiments, the antibody or antigen binding fragment conjugated to a therapeutic agent for Alzheimer's disease comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In some embodiments, the heavy chain variable region comprises an amino acid sequence of SEQ ID NO: 7 and the light chain variable region comprises an amino acid sequence of SEQ ID NO: 8. In some embodiments, the antibody or antigen binding fragment comprises any one or more (e.g., all six CDRs and/or a heavy and/or light chain variable domain) sequence selected fro those shown in Table 1. In some embodiments, the antibody or antigen binding fragment comprises a heavy chain variable region and a light chain variable region, wherein the heavy chain variable region comprises the amino acid sequence of SEQ ID NO: 43 and the light chain variable region comprises the amino acid sequence of SEQ ID NO: 44; or the heavy chain variable region comprises the amino acid sequence of SEQ ID NO: 45 and the light chain variable region comprises the amino acid sequence of SEQ ID NO: 46; or the heavy chain variable region comprises the amino acid sequence of SEQ ID NO: 47 and the light chain variable region comprises the amino acid sequence of SEQ ID NO: 48; or the heavy chain variable region comprises the amino acid sequence of SEQ ID NO: 49 and the light chain variable region comprises the amino acid sequence of SEQ ID NO: 50; or the heavy chain variable region comprises the amino acid sequence of SEQ ID NO: 51 and the light chain variable region comprises the amino acid sequence of SEQ ID NO: 52; or the heavy chain variable region comprises the amino acid sequence of SEQ ID NO: 53 and the light chain variable region comprises the amino acid sequence of SEQ ID NO: 54; or the heavy chain variable region comprises the amino acid sequence of SEQ ID NO: 55 and the light chain variable region comprises the amino acid sequence of SEQ ID NO: 56; or the heavy chain variable region comprises the amino acid sequence of SEQ ID NO: 57 and the light chain variable region comprises the amino acid sequence of SEQ ID NO: 58; or the heavy chain variable region comprises the amino acid sequence of SEQ ID NO: 59 and the light chain variable region comprises the amino acid sequence of SEQ ID NO: 60; or the heavy chain variable region comprises the amino acid sequence of SEQ ID NO: 61 and the light chain variable region comprises the amino acid sequence of SEQ ID NO: 62; or the heavy chain variable region comprises the amino acid sequence of SEQ ID NO: 63 and the light chain variable region comprises the amino acid sequence of SEQ ID NO: 64; or the heavy chain variable region comprises the amino acid sequence of SEQ ID NO: 65 and the light chain variable region comprises the amino acid sequence of SEQ ID NO: 66; or the heavy chain variable region comprises the amino acid sequence of SEQ ID NO: 67 and the light chain variable region comprises the amino acid sequence of SEQ ID NO: 68; or the heavy chain variable region comprises the amino acid sequence of SEQ ID NO: 69 and the light chain variable region comprises the amino acid sequence of SEQ ID NO: 70; or the heavy chain variable region comprises the amino acid sequence of SEQ ID NO: 71 and the light chain variable region comprises the amino acid sequence of SEQ ID NO: 72; or the heavy chain variable region comprises the amino acid sequence of SEQ ID NO: 73 and the light chain variable region comprises the amino acid sequence of SEQ ID NO: 74; or the heavy chain variable region comprises the amino acid sequence of SEQ ID NO: 75 and the light chain variable region comprises the amino acid sequence of SEQ ID NO: 76; or the heavy chain variable region comprises the amino acid sequence of SEQ ID NO: 77 and the light chain variable region comprises the amino acid sequence of SEQ ID NO: 78; or the heavy chain variable region comprises the amino acid sequence of SEQ ID NO: 79 and the light chain variable region comprises the amino acid sequence of SEQ ID NO: 80; or the heavy chain variable region comprises the amino acid sequence of SEQ ID NO: 81 and the light chain variable region comprises the amino acid sequence of SEQ ID NO: 82; or the heavy chain variable region comprises the amino acid sequence of SEQ ID NO: 83 and the light chain variable region comprises the amino acid sequence of SEQ ID NO: 84; or the heavy chain variable region comprises the amino acid sequence of SEQ ID NO: 85 and the light chain variable region comprises the amino acid sequence of SEQ ID NO: 86; or the heavy chain variable region comprises the amino acid sequence of SEQ ID NO: 87 and the light chain variable region comprises the amino acid sequence of SEQ ID NO: 88; or the heavy chain variable region comprises the amino acid sequence of SEQ ID NO: 89 and the light chain variable region comprises the amino acid sequence of SEQ ID NO: 90; or the heavy chain variable region comprises the amino acid sequence of SEQ ID NO: 91 and the light chain variable region comprises the amino acid sequence of SEQ ID NO: 92; or the heavy chain variable region comprises the amino acid sequence of SEQ ID NO: 93 and the light chain variable region comprises the amino acid sequence of SEQ ID NO: 94; or the heavy chain variable region comprises the amino acid sequence of SEQ ID NO: 95 and the light chain variable region comprises the amino acid sequence of SEQ ID NO: 96; or the heavy chain variable region comprises the amino acid sequence of SEQ ID NO: 97 and the light chain variable region comprises the amino acid sequence of SEQ ID NO: 98; or the heavy chain variable region comprises the amino acid sequence of SEQ ID NO: 57 and the light chain variable region comprises the amino acid sequence of SEQ ID NO: 58; or the heavy chain variable region comprises the amino acid sequence of SEQ ID NO: 65 and the light chain variable region comprises the amino acid sequence of SEQ ID NO: 66; or the heavy chain variable region comprises the amino acid sequence of SEQ ID NO: 77 and the light chain variable region comprises the amino acid sequence of SEQ ID NO: 78; or the heavy chain variable region comprises the amino acid sequence of SEQ ID NO: 79 and the light chain variable region comprises the amino acid sequence of SEQ ID NO: 80; or the heavy chain variable region comprises the amino acid sequence of SEQ ID NO: 83 and the light chain variable region comprises the amino acid sequence of SEQ ID NO: 84; or the heavy chain variable region comprises the amino acid sequence of SEQ ID NO: 85 and the light chain variable region comprises the amino acid sequence of SEQ ID NO: 86; or the heavy chain variable region comprises the amino acid sequence of SEQ ID NO: 91 and the light chain variable region comprises the amino acid sequence of SEQ ID NO: 92; or the heavy chain variable region comprises the amino acid sequence of SEQ ID NO: 93 and the light chain variable region comprises the amino acid sequence of SEQ ID NO: 94.

In some embodiments, the antibody or antigen binding fragment comprises SEQ ID NOS: 29 and 30. In some embodiments, the antibody or antigen binding fragment conjugated to a therapeutic agent may compete for binding or bind the same epitope as this antibody defined by sequence.

In some embodiments, the therapeutic agent may a passive immunotherapy against amyloid beta or tau, or an inhibitor of acetylcholinestarase. In some embodiments, the therapeutic agent may be selected from one or more of: CAD106, Gantenerumab, Crenezumab, IVIG, AADvac1, ACI-35, NIC5-15, CHF-5074, MK-8931, AZD 3293, LY33 14814, Elenbecestat, Tideglusib, Intranasal Humulin R, Intensal glulisine, SB742457 with donepezil, Azeliragon, Nivaldipine. See Godyn et al., Pharmacological Reports, 68:127-138, 2016. In some embodiments, the therapeutic agent may be selected from: Aducanumab, ALZT-OP1a+ALZT-OP1b, Aripiprazole, AVP-786, AZD3293 (LY3314814), Brexprprazole (OPC-34712), CAD106, CNP520, Elenbecestat, Insulin (humulin), Lumateperone, JNJ-54861911, Methylphenidate, MK-4305 (suvorexant), Nabilone, Nilvadipine, Pioglitazone, RVT-101 (intepirdine), Sodium Oligo-mannurarate (GV-971), TRx0237, TTp488 (azeliragon), AADvac1, ABBV-8E12, AD-SVF cells, ANAVEX 2-73, Atomoxetine, AVP-786, AZD0530 (saracatinib), BAC, BAN2401, Benfotiamine, B1409306, Bryostatin 1, Candesartan, CB-AC-02, Cilostazol, CPC-201, CT1812, DAOIB, Dronabinol, E2609, Formoterol, hUCB-MSCs, JNJ-54861991, Levetiracetam, Liraglutide, LY3202626, NewGam 10% IVIG, Nilotinib, ORM-12741, Pimavanserin, Piromelatine, Posiphen, PQ912, Probucol, Rasagiline, Riluzole, RVT-101, S47445, Sargramostim, Simvastatin+L-Arginine+Tetrahydrobiopterin (SLAT), STA-1, SUVN-502, T-817 MA, Temisartan, UB-311, Valacyclovir, VX-745, Xanamema. See Cummings et al., Alzheimer's disease drug development pipeline: 2017, Alzheimer's & Dementia, 3:367-384, 2017. In some embodiments, the anti-tau therapy comprises a small molecule or peptide vaccine therapy, or an anti-tau antibody therapy. See U.S. Pat. No. 9,518,101, which is incorporated by reference in its entirety.

In further embodiments, an immunoconjugate is provided having the formula (A)-(L)-(C), wherein: (A) is an antibody or antigen binding fragment thereof disclosed herein; (L) is an optional linker; and (C) is a second agent (e.g., a detectable label or a therapeutic agent for treating AD or another tauopathy); wherein the linker (L) joins (A) to (C). In some embodiments, (C) is a therapeutic agent, an imaging agent, a detectable agent, or a diagnostic agent. In some embodiments, these conjugates are referred to herein as antibody-drug-conjugates (ADCs).

Optional linker (L), as used herein, may be present or absent. When present, (L) is a molecule that is used to join the (A) to (C). In some embodiments, the linker is capable of forming covalent bonds to both the antibody and to the second agent. Suitable linkers are well known to those of skill in the art and include, but are not limited to, straight or branched-chain carbon linkers, heterocyclic carbon linkers, or peptide linkers. Where the antibody and second agent are polypeptides, the linkers may be joined to the constituent amino acids through their side groups (e.g., through a disulfide linkage to cysteine). However, in another embodiment, the linkers may be joined to the alpha carbon amino and carboxyl groups of the terminal amino acids.

In some circumstances, it is desirable to free the second agent from the antibody when the immunoconjugate has reached its target site. Therefore, in these circumstances, immunoconjugates may comprise linkages which are cleavable in the vicinity of the target site. Cleavage of the linker to release the second agent from the antibody may be prompted by enzymatic activity or conditions to which the immunoconjugate is subjected either inside the target cell or in the vicinity of the target site. In yet other embodiments, the linker unit is not cleavable and the drug is not released or is released, for example, by antibody degradation.

A number of different reactions are available for covalent attachment of drugs and/or linkers to antibodies or antigen binding fragments thereof. This is often accomplished by reaction of the amino acid residues of the antibody molecule, including the amine groups of lysine, the free carboxylic acid groups of glutamic and aspartic acid, the sulfhydryl groups of cysteine and various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule. Also available for attachment of drugs to antibodies is the Schiff base reaction. This method involves the periodate oxidation of a drug that contains glycol or hydroxyl groups, thus forming an aldehyde which is then reacted with the binding agent. Attachment occurs via formation of a Schiff base with amino groups of the binding agent.

Isothiocyanates can also be used as coupling agents for covalently attaching drugs to binding agents.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. See, e.g., Dubowchik and Walker, 1999, Pharm. Therapeutics 83:67-123. Other examples of such linkers are described, e.g., in U.S. Pat. No. 6,214,345. In other embodiments, the cleavable linker is pH-sensitive, i.e., sensitive to hydrolysis at certain pH values. Typically, the pH sensitive linker is hydrolysable under acidic conditions. For example, an acid-labile linker that is hydrolysable in the lysosome (e.g., a hydrozone, semicarbazone, thiosemicarbazone, cis-aconitic aminde, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha(2 pyridyl-dithio)toluene)-, SPDB and SMPT. (See, e.g., Thorpe et al., 1987, Cancer Res. 47:5924-5931; Wawrzynczak et al., In Immunoconjugates: Antibody Conjugates in Radiolmagery and Therapy of Cancer (C. W. Vogel ed., Oxford U. Press, 1987. See also U.S. Pat. No. 4,880,935.) In another embodiment, the linker is a malonate linker (Johnson et al., 1995, Anticancer Res. 15:1387-93), a maleimidobenzoyl linker (Lau et al., 1995, Bioorg-Med-Chem. 3(10): 1299-1304), or a 3′-N-amide analog (Laur et al., 1995, Bioorg-Med-Chem. 3(10): 1305-12).

In another embodiment, the linker unit is not cleavable and the drug is released by antibody degradation. (See U.S. Publication No. 2005/0238649). In one embodiment, the linker is not substantially sensitive to the extracellular environment. As used herein, “not substantially sensitive to the extracellular environment,” in the context of a linker, means that no more than about 20%, about 15%, about 10%, about 5%, about 3%, or no more than 1% of the linkers, in a sample of antibody-drug conjugate compound, are cleaved when the antibody-drug conjugate compound presents in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and quantifying the amount of free drug present in the plasma.

In some embodiments, (L) can also comprise a spacer group ora linkage group such as polyaldehyde, glutaraldehyde, ethylenediaminetetraacetic acid (EDTA) or diethylenetriaminepentaacetic acid (DPTA).

In some embodiments, more than one second agent may be attached, either directly or by a linker, to an antibody or antigen binding fragment disclosed herein. In certain embodiments, the ratio of second agent to antibody can range on average from about 1:1 to about 1:8. See U.S. Pat. No. 7,498,298. The loading (drug/antibody ratio) of an ADC may be controlled in different ways. See WO2006/034488.

### Nucleic Acids, Vectors, Host Cells

Also disclosed are isolated nucleic acid(s) encoding at least one variable region of an immunoglobulin chain of any of the antibodies or antigen binding fragments described herein. In certain embodiments, an isolated nucleic acid(s) encodes the antibody or antigen binding fragment of any one of the previously described antibodies or antigen binding fragments. Other embodiments provide an isolated vector comprising the nucleic acids. Another embodiment includes an isolated host cell comprising any of the nucleic acids or vectors.

The polynucleotides or nucleic acids encoding the antibodies or antigen binding fragments described herein can be, e.g., DNA, cDNA, RNA or synthetically produced DNA or RNA or a recombinantly produced chimeric nucleic acid molecule comprising any of those polynucleotides either alone or in combination. In some embodiments, the polynucleotide is part of a vector. Such vectors can comprise further genes such as marker genes and/or control elements, allowing for the selection and/or expression of the vector in a suitable host cell and under suitable conditions.

In some embodiments, the polynucleotide is operatively linked to one or more expression control sequences, allowing expression in prokaryotic or eukaryotic cells. Expression of the polynucleotide may comprise transcription of the polynucleotide into translatable mRNA. Regulatory elements ensuring expression in eukaryotic cells, for example mammalian cells, are known to those skilled in the art. They usually comprise regulatory sequences ensuring initiation of transcription and optionally poly-A signals ensuring termination of transcription and stabilization of the transcript. Additional regulatory elements can include transcriptional as well as translational enhancers, and/or naturally associated or heterologous promoter regions.

In this respect, the person skilled in the art will appreciate that the polynucleotides encoding at least the CDRs and/or variable domain of the light and/or heavy chain can encode the variable domains of both immunoglobulin chains or only one. Likewise, the polynucleotides can be under the control of the same promoter or can be separately controlled for expression. Possible regulatory elements permitting expression in prokaryotic host cells comprise, e.g., the PL, lac, trp or tac promoter in E. coli, and examples for regulatory elements permitting expression in eukaryotic host cells are the AOXI of GAL1 promoter in yeast or the CMV-, SV40-, RSV-promoter, CMV-enhancer, SV40-enhancer or a globin intron in mammalian and other animal cells.

Beside elements that are responsible for the initiation of transcription, such regulatory elements can also comprise transcription termination signals, such as the SV40-poly-A site or the tk-poly-A site, downstream of the polynucleotide. Furthermore, depending on the expression system used, leader sequences capable of directing the polypeptide to a cellular compartment or secreting it into the medium can be added to the coding sequence of the polynucleotides and are known in the art. When used, the leader sequence(s) may be assembled in appropriate phase with translation, initiation and termination sequences, and optionally, a leader sequence capable of directing secretion of translated protein, or a portion thereof, into the periplasmic space or extracellular medium. In some embodiments, the heterologous sequence can encode a fusion protein including a C- or N-terminal identification peptide imparting desired characteristics, e.g., stabilization or simplified purification of expressed recombinant product. In this context, suitable expression vectors are known in the art, and include, without limitation, the Okayama-Berg cDNA expression vector pcDV1 (Pharmacia), pCDM8, pRc/CMV, PcDNA1, PcDNA3 (Invitrogen), and pSPORT1 (GIBCO BRL).

In some embodiments, the expression control sequences can be eukaryotic promoter systems in vectors capable of transforming or transfecting eukaryotic host cells, but control sequences for prokaryotic hosts can also be used. Once the vector has been incorporated into the appropriate host, the host is maintained under conditions suitable for high level expression of nucleotide sequences, and, as desired, the collection and purification of the immunoglobulin light chains, heavy chains, light/heavy dimers or intact antibodies, binding fragments or other immunoglobulin forms can follow. See, e.g., Beychok, Cells of Immunoglobulin Synthesis, Academic Press, N.Y., (1979).

In various embodiments, vectors, particularly plasmids, cosmids, viruses, and bacteriophages can be used that comprise a polynucleotide encoding a variable domain of an immunoglobulin chain of an antibody of the present disclosure; optionally in combination with another polynucleotide that encodes the variable domain of the other immunoglobulin chain of an antibody of the present disclosure. In some embodiments, the vector is an expression vector and/or gene transfer or targeting vector. Expression vectors derived from viruses such as retrovirus, vaccina virus, adeno-associated virus, herpes virus, or bovine papilloma virus, can be used for delivery of the polynucleotides or vector of the present disclosure into targeted cell populations. Any methods that are known to those skilled in the art can be used to construct recombinant viral vectors. Alternatively, the polynucleotides and vectors provided by the invention can be reconstituted into liposomes for deliver to target cells. The vector(s) containing the polynucleotides of the present disclosure (e.g., the heavy and/or light variable domain(s) of the immunoglobulin chains encoding sequences) can be transferred into a host cell by known methods, which vary depending on the type of cellular host. For example, calcium chloride transfection is commonly utilized for prokaryotic cells, whereas calcium phosphate treatment or electroporation can be used for other cellular hosts.

In some embodiments, a host cell is transformed with a polynucleotide or vector described herein. The host cell can be a prokaryotic or eukaryotic cell. The poly nucleotide or vector that is present in the host cell can either be integrated into the genome of the host cell or it can be maintained extrachromosomally. The host cell can be any prokaryotic or eukaryotic cell, such as a bacterial, insect, fungal, plant, animal or human cell. Preferred fungal cells are, for example, those of the genus Saccharomyces, such as S. cerevisiae. Depending upon the host employed in a recombinant production procedure, the antibodies or antigen binging fragments thereof encoded by the polynucleotide can be glycosylated. Certain antibodies or antigen binding fragments thereof consistent with the present disclosure can also include an initial methionine amino acid residue. A polynucleotide disclosed herein can be used to transform or transfect the host using any of the techniques commonly known to those of ordinary skill in the art. Furthermore, methods for preparing fused, operably linked genes and expressing them in, e.g., mammalian cells and bacteria are well-known in the art. In general, expression vectors containing promoter sequences which facilitate the efficient transcription of the inserted polynucleotide are use in connection with a host. The expression vector typically contains an origin of replication, a promoter, and a terminator, as well as specific genes which are capable of providing phenotypic selection of the transformed cells. Suitable source cells for DNA sequences and host cells for immunoglobulin expression and secretion can be obtained from a number of sources, such as the American Type Culture Collection (“Catalogue of Cell Lines and Hybridomas,” Fifth edition (1985) Manassas, Va., U.S.A., and other available version, incorporated herein by reference). Furthermore, transgenic animals, for example mammals, comprising cells of the invention can be used for large scale production of the antibodies or antigen binding fragments disclosed herein.

In another embodiment, a hybridoma producing antibody DC2E7 is disclosed. The hybridoma has been deposited at the American Type Culture Collection at Patent Deposit No. PTA-124992.

In another embodiment, a hybridoma producing antibody DC2E2 is disclosed. The hybridoma has been deposited at the American Type Culture Collection at Patent Deposit No. PTA-124991.

### Compositions, Formulations and Combinations

In some exemplary embodiments, an antibody or antigen binding fragment thereof described herein may be co-formulated and/or co-administered with one or more additional compounds that are also useful in the detection, prevention, and/or treatment of AD or another tauopathy.

In some embodiments, an antibody or antigen binding fragment thereof described herein is formulated for use in an assay to detect phosphorylated tau in a biologic sample from a human subject (e.g., CSF or blood). In certain embodiments, the antibody or antigen binding fragment thereof is conjugated to a detectable label, e.g., an enzyme, a radioisotope, a fluorophore, a nuclear magnetic resonance marker, or a heavy metal. In some embodiments, two or more (e.g., 3, 4, 5, etc.) antibodies or antigen binding fragments described herein are formulated suitable for use in the assay. In certain embodiments, the antibodies or antigen binding fragments comprise antibody clones DC2E7 or DC2E2 and/or antibodies or fragments comprising the CDRs or variable domains from those clones, alone or conjugated to suitable detectable labels. In some embodiments, the formulations further comprise additional elements and reagents (e.g., solid supports or particles such as magnetic beads) suitable for use in diagnostic assays such as the classic and digital ELISA assays described below.

In some embodiments, the antibodies and antigen binding fragments described herein are prepared for use in methods of detecting phosphorylated tau in a biologic sample. For instance, the antibodies can be prepared and formulated as discussed in Example 3 below. In some embodiments, the antibodies or antigen binding fragments, e.g., DC2E7, DC149, DC807, and/or DC2E2 or antigen binding fragments or variants thereof, may be purified from serum-free hybridoma supernatant, for example using a Protein G affinity column. The purified supernatant can then be eluted, e.g, with 0.1 M Glycine-HCl, pH 2.7 and neutralized with 1M Tris-HCl pH 9.0. Pooled fractions can be diazlied in a buffer solution such as phosphate buffered saline (PBS). In some embodiments, the buffered antibody solution can be concentrated, e.g., by ultrafiltration. In some embodiments, antibody concentration is determined by measuring absorbance at 280 nm, using the formula c (mg/ml)=A280 nm/1.43.

DC2E7 or an antigen binding fragment, when used as the capture antibody in the digital ELISA, may be prepared in a suitable buffer solution. In some embodiments, DC2E7 or an antigen binding fragment is immobilized on a solid support (e.g., a solid surface or ELISA capture bead). In some embodiments, DC2E7 or an antigen binding fragment is joined to a solid surface, e.g., as described according to Example 10 below. Briefly, DC2E7 may be coupled to a bead, e.g, a magnetic bead (Quanterix). In some embodiments, DC2E7 is coupled to the bead at a concentration of about 0.1-5.0 mg/mL (e.g., about 1.0 mg/mL). In some embodiments, DC2E2 or an antigen binding fragment is used as a detector antibody and may be conjugated to a detectable label. For instance, the antibody or antigen binding fragment may be biotinylated for detection purposes, e.g., by exposure to 1-200 fold (e.g., about 120 fold) excess of biotin relative to the antibody concentration.

In some embodiments, an antibody or antigen binding fragment thereof described herein may be co-formulated and/or co-administered with a detectable label. In some embodiments, the detectable label is an enzyme, a radioisotope, a fluorophore, a nuclear magnetic resonance marker, or a heavy metal. In some embodiments, the detectable label is in association (e.g., covalent or non-covalent) association with the antibody or antigen binding fragment. Suitable additives and formulation conditions for antibody administration may be used, e.g., those known in the art for formulating antibodies for parenteral administration (e.g., intravenous, subcutaneous, intraperitoneal, intramuscular), or intravenous, intramuscular or subcutaneous injection.

In some embodiments, an antibody or antigen binding fragment thereof is formulated in combination with additional compounds that are also useful in the prevention and/or treatment of AD or another tauopathy. These include, without limitation, compounds that are useful in active and passive immunotherapies for AD, such as beta-amyloid peptides (e.g., N-terminal amyloid beta peptides) and tau peptides which might or might not be conjugated to other compounds, such as mutated diphtheria toxin, KLH or other carriers. Other options include antibodies against beta-amyloid, such as bapineuzumab, solaneuzumab, gantenerumab, crenezumab, ponezumab, and IVIG immunoglobulin, other immunization therapies targeting Abeta oligomers, other tau antibodies, compounds preventing the hyperphosphorylation of tau, and other active and passive immunization therapies targeting tau aggregates.

Other drugs that may be helpful in combination therapy with the antibodies and tau-binding fragments described herein include amyloid-beta aggregation inhibitors (e.g., Tramiprosate), gamma-secretase inhibitors (e.g., semagacestat), and gamma-secretase modulators (tarenflurbil). Furthermore, one or more of the novel antibodies disclosed herein may be used or formulated in combination with two or more of the foregoing therapeutic agents. At early stages of the disease, combination therapies can be advantageous. Combination therapies are also advantageous at later stages of the disease, such as combination of hAb and growth factors and other biologically active molecules inducing neuronal plasticity and regeneration. Such combination therapies may advantageously utilize lower dosages of the administered therapeutic agents, thus avoiding possible toxicities or complications associated with the various monotherapies. According to a related embodiment, an antibody or antigen binding fragment thereof described herein can be used in combination with at least one combination agent chosen from acetylcholinesterase inhibitors (e.g., donepezil, rivastigmine, galantamine, tacrine, nutritive supplements), N-Methyl-D-aspartate (NMDA) receptor antagonists (e.g., memantine), inhibitors of DNA repair (e.g., pirenzepine or a metabolite thereof), transition metal chelators, growth factors, hormones, non-steroidal anti-inflammatory drugs (NSAID), antioxidants, lipid lowering agents, selective phosphodiesterase inhibitors, inhibitors of tau aggregation, inhibitors of protein kinases, inhibitors of anti-mitochondrial dysfunction drugs, neurotrophins, inhibitors of heat shock proteins, inhibitors of Lipoprotein-associated phospholipase A₂, and any pharmaceutically acceptable salts thereof. In one embodiment, a disclosed antibody and/or tau-binding fragment thereof is combined with a cholinesterase inhibitor (ChEI) and/or memantine. In one embodiment, the combination agent is selected from the group consisting of an anti-apoptotic compound, a metal chelator, an inhibitor of DNA repair, 3-amino-1-propanesulfonic acid (3APS), 1,3-propanedisulfonate (1,3PDS), a secretase activator, a beta-secretase inhibitor, a gamma-secretase inhibitor, a beta-amyloid peptide, a beta-amyloid antibody, a neurotransmitter, a beta-sheet breaker, an anti-inflammatory molecule, and a cholinesterase inhibitor. In one embodiment, the cholinesterase inhibitor is, rivastigmine, donepezil, galantamine, or a nutritive supplement. In another embodiment, the additional agent is selected from BACE inhibitors; muscarinic antagonists; cholinesterase inhibitors; gamma secretase inhibitors; gamma secretase modulators; HMG-CoA reductase inhibitors; nonsteroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABAA inverse agonists; inhibitors of amyloid aggregation; glycogen synthase kinase beta inhibitors; promotors of alpha secretase activity; PDE-10 inhibitors and cholesterol absorption inhibitors.

Other compounds that may be used in combination with the antibody and antigen binding fragment described herein include the therapeutic antibodies and peptides described in U.S. Pat. No. 9,518,101, which is incorporated herein by reference in its entirety. Also useful are the compounds described in WO 2004/058258, which is incorporated herein by reference in its entirety (see especially pages 16 and 17) including therapeutic drug targets (page 36-39), alkanesulfonic acids and alkanolsulfuric acids (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferators-activated receptor (PPAR) agonists (pages 63-67), cholesterol-lowering agents (pages 68-75); amyloid inhibitors (pages 75-77), amyloid formation inhibitors (pages 77-78), metal chelators (pages 78-79), antipsychotics and anti-depressants (pages 80-82), nutritional supplements (pages 83-89) and compounds increasing the availability of biologically active substances in the brain (see pages 89-93) and prodrugs (pages 93 and 94). Other compounds that may be used in combinations include those described in Cummings et al., Alzheimer's disease drug development pipeline: 2017, Alzheimer's & Dementia: Translational Research & Clinical Interventions 3 (2017) 367-384.

Also provided herein are compositions comprising an antibody or an antigen binding fragment thereof, as described herein, and another component, such as a carrier. Also provided are compositions/formulations comprising a humanized antibody or antigen binding fragment thereof, as described herein, and a carrier, e.g., a carrier suitable for diagnostic or therapeutic uses.

In one embodiment, formulations and compositions of the antibodies used in accordance with the present disclosure are prepared for storage and/or administration by mixing an antibody or antigen binding fragment thereof having the desired degree of purity with optional carriers, e.g., pharmaceutically acceptable carriers, diluents, excipients or stabilizers (Remington's Pharmaceutical Sciences 21^{st} edition, Mohr, M. Ed. (2006)), in the form of lyophilized formulations or aqueous solutions. In one embodiment, acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbezyl ammonium chloride; hexamethonium chloride; benzalkonium chloride, benzethonium chloride, phenol, butyl or benyl alcholol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium, metal complexes (e.g., Zn-protein complexes); and/or non-ionic surfactants such as TWEEN, PLURONICS or polyethylene glycol (PEG). Examples of lyophilized antibody formulations are described in WO 97/04801, expressly incorporated herein by reference.

In one embodiment, the antibodies and antigen binding fragments thereof can be incorporated into pharmaceutical compositions suitable for administration to a subject. In certain embodiments, the pharmaceutical composition comprises an antibody or antigen binding fragment thereof as disclosed herein and a pharmaceutically acceptable carrier. In one embodiment, “pharmaceutically acceptable carrier” includes any and all solvents, dispersion media, coatings, antibacterial and antifungal agents, isotonic and absorption delaying agents, and the like that are physiologically compatible. Additional examples of pharmaceutically acceptable carriers include one or more of water, saline, phosphate buffered saline, dextrose, glycerol, ethanol and the like, as well as combinations thereof. In many cases, the composition will include isotonic agents, for example, sugars, polyalcohols such as mannitol, sorbitol, or sodium chloride. Pharmaceutically acceptable carriers may further comprise minor amounts of auxiliary substances such as wetting or emulsifying agents, preservatives or buffers, which enhance the shelf life or effectiveness of the antibody or antigen binding fragments thereof.

The compositions described herein, comprising the disclosed antibodies or antigen binding fragments, may be in a variety of forms. These include, for example, liquid, semi-liquid, semi-solid and solid dosage forms, such as liquid solutions (e.g., injectable and infusible solutions), dispersions or suspensions, tablets, pills, powders, liposomes and suppositories. In some embodiments, such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Antibodies and antigen binding fragments thereof may also be encapsulated within liposomes.

Dosage forms suitable for use in diagnostic assays or for internal administration generally contain from about 0.1 milligram to about 500 milligrams of antibody or antigen binding fragment thereof per unit or container. In these compositions, the active ingredient will ordinarily be present in an amount of about 0.5-99.999% by weight based on total weight of the composition. The preferred dosage form depends on the intended use and/or mode of administration.

In certain embodiments, the antibodies or antigen binding fragments disclosed herein can traverse the blood-brain barrier or are formulated to traverse the blood-brain barrier. Certain neurodegenerative diseases, including AD and related tauopathies, are associated with an increase in permeability of the blood-brain barrier, such that the antibody or antigen binding fragment thereof can be readily introduced into the brain. When the blood-brain barrier remains intact, several art-known approaches exist for transporting molecules across it, including, but not limited to, physical methods, lipid-based methods, and receptor and channel-based methods. Methods for circumventing the blood-brain barrier include, but are not limited to, direct injection into the brain (see, e.g., Papanastassiou et al., Gene Therapy 9: 398-406 (2002)) and implanting a delivery device in the brain (see, e.g., Gill et al., Nature Med. 9: 589-595 (2003): and Gliadel Wafers, T. M., Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the antibody or antigen binding fragment thereof (see, e.g., U.S. Patent Publication No. 2003/0083299). Lipid-based methods of transporting the antibody or antigen binding fragment thereof across the blood-brain barrier include, but are not limited to, encapsulating the antibody or antigen binding fragment thereof in liposomes that are coupled to active fragments thereof that bind to receptors on the vascular endothelium of the blood-brain barrier (see, e.g., U.S. Patent Application Publication No. 20020025313), and coating the antibody or antigen binding fragment thereof in low-density lipoprotein particles (see, e.g., U.S. Patent Application Publication No. 20040204354) or apolipoprotein E (see, e.g., U.S. Patent Application Publication No. 20040131692).

In various embodiments, a composition can comprise any one or more antibodies or antigen binding fragments described herein and a carrier and/or diluent. In some embodiments, the antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In some embodiments, the antibody or antigen binding fragment comprises any one or more (e.g., all six CDRs and/or a heavy and/or light chain variable domain) sequence selected fro those shown in Table 1. In some embodiments, the antibody or antigen binding fragment comprises any of the sequences shown in table 1, e.g., a set of six CDRs and or paired heavy and light chain variable domain sequences listed in Table 1. In some embodiments, the antibody or antigen binding fragment comprises SEQ ID NOS: 29 and 30. In some embodiments, the composition is suitable for use in a diagnostic assay, e.g., classic or digital ELISA. In some embodiments, the composition is a pharmaceutical composition and comprises a pharmaceutically-acceptable carrier.

In certain embodiments, a composition comprises any two antibodies or antigen binding fragments as previously described. In certain embodiments, a composition comprises at least one further antibody or antigen binding fragment, and/or at least one additional agent. In an exemplary embodiment, one antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; and the other antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20.

In some embodiments, the first antibody or antigen binding fragment comprises any one or more (e.g., all six CDRs and/or a heavy and/or light chain variable domain) sequence selected fro those shown in Table 1. In some embodiments, the antibody or antigen binding fragment comprises any of the sequences shown in table 1, e.g., a set of six CDRs and or paired heavy and light chain variable domain sequences listed in Table 1. In some embodiments, the antibody or antigen binding fragment comprises SEQ ID NOS: 29 and 30. In some embodiments, the second antibody or antigen binding fragment comprises any one or more (e.g., all six CDRs and/or a heavy and/or light chain variable domain) sequence selected fro those shown in Table 1. In some embodiments, the antibody or antigen binding fragment comprises any of the sequences shown in table 1, e.g., a set of six CDRs and or paired heavy and light chain variable domain sequences listed in Table 1. In some embodiments, the antibody or antigen binding fragment comprises SEQ ID NOS: 29 and 30.

In some embodiments, the composition is suitable for use in a diagnostic assay, e.g., classic or digital ELISA. In some embodiments, the composition is a pharmaceutical composition and comprises a pharmaceutically-acceptable carrier. The pharmaceutical composition may further comprise at least one additional therapeutic agent for treating Alzheimer's disease or another tauopathy.

### Methods of Treatment

The antibodies and compositions described herein can be used for various methods of treatment and prevention of AD and other tauopathies. In some embodiments, the antibodies and compositions can be used to detect patients suitable for anti-tau based treatments, to inform treatment selection, or to monitor the progress of treatment over time. In other embodiments, the antibodies and compositions disclosed herein can be used as direct treatments or preventions for AD and other tauopathies by administering one or more antibodies or compositions to a patient in need thereof.

In preventative (i.e., prophylactic) applications, the antibodies and pharmaceutical compositions disclosed herein (e.g., antibody DC2E7 or an antibody or antigen binding fragment that can bind the same epitope as, or comprises the CDRs or variable domains from, antibody DC2E7) may be administered to a patient susceptible to, or otherwise at risk of, Alzheimer's disease or another tauopathy. In some embodiments, the antibody or pharmaceutical composition is administered in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions are administered to a patient suspected of, diagnosed with, and/or already suffering from such a disease in an amount sufficient to cure, or at least partially reduce, arrest, or reverse the symptoms of the disease (biochemical, histologic, and/or behavioral), including its complications and intermediate pathological phenotypes in development of the disease.

In some embodiments, treating Alzheimer's disease refers to decreasing or preventing behavioral, functional, and cognitive deterioration over time. In some embodiments, behavioral, functional, and cognitive aspects of Alzheimer's Disease can be evaluated by any one or more of a series of standardized tests known to persons of ordinary skill in the art including, but not limited to, neuropsychological testing, the Mini-Mental State Exam, Mini-cog exam, Neuropsychiatric Inventory, Blessed Roth Dementia Rating Scale, Spanish and English Neuropsychoiogical Assessment Scales (SENAS), Psychiatric Behavioral Assessment, Functional Assessment, Clock Drawing Test, Boston Naming Test. California Verbal Learning Test, Cognitive Symptoms Checklist, Continuous Performance Test, Controlled Oral Word Association Test, Cognistat, d2 Test of Attention, Delis-Kaplan Executive Function System, Dementia Rating Scale, Digit Vigilance Test, Figural Fluency Test, Finger Tapping Test, Halstead Category Test, Halstead-Reitan Neuropsychological Battery, Hooper Visual Organization Test, Kaplan Baycrest Neurocognitive Assessment, Kaufman Short Neuropsychological Assessment, Luria-Nebraska Neuropsychological Battery, Memory Assessment Scales, Quick Neurological Screening Test, Repeatable Battery for the Assessment of Neuropsychological Status, Stroop Test, Symbol Digit Modalities Test, Tactual Performance Test, Thematic Apperception Test, Tower of London, Trail Making Tests A and B, Verbal (Word) Fluency Tests, and Wisconsin Card Sort Test. Additional tests for depression, anxiety, aphasia, agitation, and behavioral parameters known to persons of ordinary skill in the art are also used. In addition, in some embodiments, patients may be diagnosed or monitored for treatment efficacy using any of the detection methods disclosed herein involving one or more of the disclosed antibodies or antigen binding fragments thereof.

In some embodiments, the antibodies and pharmaceutical compositions disclosed herein (e.g., antibody DC2E7 or an antibody or antigen binding fragment that can bind the same epitope as, or comprises the CDRs or variable domains from, antibody DC2E7) may be administered to treat AD or another tauopathy in a patient. In some embodiments, the efficacy of an Alzheimer's disease treatment is determined by the improvement, or lack of deterioration, or a reduction in the rate of deterioration in at least one assessment selected from the group consisting of Alzheimer's Disease Assessment Scale-cognitive subscale (ADAS-cog), the Clinical Dementia Rating Sum of Boxes (CDR-sb), the Alzheimer's Disease Cooperative Study Activities of Daily Living Scale (ADCS-ADL), the Neuropsychiatric inventory (NPI), the Progressive Deterioration Scale (PDS), Amsterdam Instrumental Activities of Daily Living (IADL), the Clinical Dementia Rating Scale (CDR), the Disability Assessment for Dementia Scale (DAD), and the Mini-Mental State Evaluation (MMSE). In some embodiments, the treatment results in a reduction in the rate of deterioration in ADAS-cog scores. In other embodiments, the treatment results in a median reduction in the rate of deterioration of ADAS-cog scores of two to five points.

In some embodiments, a method of delaying the progression of Alzheimer's disease is provided, comprising administering one or more of the disclosed antibodies, e.g., antibody DC2E7 or an antibody or antigen binding fragment that binds the same epitope as, or comprises the CDRs or variable domains from, antibody DC2E7. In one embodiment, “delaying” progression of AD means to defer, hinder, slow, retard, stabilize, and/or postpone development of the disease. This delay can be for a varying length of time, depending on the history of the disease and/or individual being treated. As is evident to one skilled in the art, a sufficient or significant delay can, in effect, encompass prevention, in that the individual does not develop the disease. In one embodiment, a method that “delays” progression of AD or another tauopathy is a method that reduces the probability of disease development in a given time frame and/or reduces extent of the disease in a given time frame, when compared to not using the method. Such comparisons are typically based on clinical studies, using a statistically significant number of subjects.

In certain embodiments, AD or another tauopathy may be delayed by days, months, or years. For example, the method may delay progression of AD or another tauopathy by one or more weeks, months, or years.

Patients, subjects, or individuals include mammals, such as human, bovine, equine, canine, feline, porcine, and ovine animals. The subject may be a human, and may or may not be afflicted with disease or presently show symptoms. In the case of AD, virtually anyone is at risk of suffering from AD if he or she lives long enough. Therefore, the present methods may be administered prophylactically to the general population without the need for any assessment of the risk of the subject patient. In other embodiments, the subject is assessed for AD using a detection method disclosed herein.

In one embodiment, the patient herein is optionally subjected to a diagnostic test prior to therapy, which may include the diagnostic methods disclosed herein. In one embodiment, the disclosed methods are useful for individuals who have a known genetic risk of AD. Such individuals include those having relatives who have experienced this disease and those whose risk is determined by analysis of genetic or biochemical markers. Genetic markers of risk towards AD include mutations in the APP gene, particularly mutations at position 717 and positions 670 and 671 referred to as the Hardy and Swedish mutations, respectively. See Hardy (1997) Trends Neurosci. 20:154-9). Other markers of risk are mutations in the presenilin genes, PS1 PS2, and ApoE4, family history of AD, hypercholesterolemia or atherosclerosis. Individuals presently suffering from AD may also be identified from behavioral characteristics. In asymptomatic patients, treatment can begin at any age (e.g., 10, 20, 30). In some patients, it may not be necessary to begin treatment and/or monitoring until a patient reaches an older age, e.g., 40, 50, 60, or 70, or later, or any time period in between. Treatment may entail multiple dosages over a period of time. Treatment can be monitored in various ways, including by using the AD detection methods disclosed herein.

Periodic use of one or more of these tests can advise a physician or other medical professional as to the progression, or regression of Alzheimer's disease and related tauopathies and the need for further treatment. The choice of test and the determination of success of treatment are within the expertise of medical professionals in the Alzheimer's disease field. An improved score in one or more tests is an indication of decrease in severity of AD in that subject.

In various embodiments, a method of treating, delaying progression, or preventing the progression of Alzheimer's disease of another tauopathy in a subject is disclosed, comprising administering to the subject an effective amount of at least one antibody or antigen binding fragment described herein. This method may result in reducing motor impairment, improving motor function, reducing cognitive impairment, improving cognitive function, or a combination thereof. The use of any antibody described herein may be used in treating, delaying progression, or preventing Alzheimer's disease by administering the antibody or antigen binding fragment to a subject in need thereof.

### Methods of Detecting and Monitoring Progression of AD and Other Tauopathies

The disclosure herein focuses, in part, on the discovery of certain epitope residues on tau, particularly those containing one or more phosphorylated residues on tau, that can be detected in certain biological samples (e.g., blood or CSF) and used to identify and distinguish AD, other tauopathies, and other forms of dementia. For example, the useful epitope residues may comprise one or more of residues 188-227 of tau protein 2N4R (SEQ ID NO: 10). In certain embodiments, the epitope comprises one or more of residues 210-221 of tau protein 2N4R (SEQ ID NO: 11). In certain embodiments, the epitope comprises at least one phosphorylated residue, e.g., phospho-threonine at position 217 of tau protein 2N4R (SEQ ID NO: 9), and optionally also comprises a phosphorylated serine at position 210, threonine at position 212, serine at position 214, or threonine at position 220 of tau protein 2N4R, or any combination thereof. In another embodiment, the epitope comprises or consists of SRTPSLPpTPPTR (sequence of SEQ ID NO: 12) or that stretch of amino acid residues with one or more additional phophorylated positions in it. In another embodiment, the epitope comprises or consists of SRpTPSLPpTPPTR (sequence of SEQ ID NO: 31). The disclosure is based in part on the surprising finding that the amount of phosphorylated tau species in these samples can be used to distinguish AD from other tauopathies and from subjects with other forms of dementia, and thereby diagnose, monitor, and/or guide treatment decisions for AD or for another tauopathy or for another cause of dementia based on the results of the assay. Without being bound by theory, the discovery that the level of these phosphorylated epitopes (e.g., at position 217) can be used to distinguish AD, other tauopathies, and other forms of dementia in certain samples is particularly unexpected given the fact that the phosphorylated epitopes can often be detected in the brain across different tauopathies.

The disclosure provided herein also focuses, in part, on antibodies (e.g., any antibodies comprising one or more sequences shown in Table 1, e.g., all six CDRs and/or a heavy and/or light chain variable domain selected from those shown in Table 1) that are capable of binding to these particular phosphorylated tau epitopes in certain types of biological samples (e.g., CSF or blood) and are particularly useful for identifying and distinguishing AD, other tauopathies, and other forms of dementia based on the level of binding in the samples. The disclosure is based in part on the surprising finding that the amount of phosphorylated tau species bound by the disclosed antibodies in these samples can be used to distinguish AD from other tauopathies and from subjects with other forms of dementia, and thereby diagnose, monitor, and/or guide treatment decisions for AD or for another tauopathy or for another cause of dementia based on the results of the assay.

In addition to AD, other tauopathies are known in the art, including frontotemporal dementia (FTD), corticobasal disease (CBD), and progressive supranuclear palsy (PSP). Murray et al., Clinicopathologic assessment and imaging of tauopathies in neurodegenerative dementias, Alzheimer's Research & Therapy, 6:1, 2014. Numerous other causes of dementia are also known. Without being bound by theory, the elevated amounts of the phosphorylated tau species in certain biological samples from AD patients and patients with other tauopathies (e.g., CSF or blood) that are bound by the antibodies or antigen binding fragments disclosed herein may contribute to their usefulness in non-invasively detecting and distinguishing AD from other causes of dementia or other tauopathies in these bodily fluids.

Numerous tau species are present in certain types of samples (e.g., blood and/or CSF) from both healthy subjects and those with dementia. Despite these many potential targets, antibodies that can bind to particular tau species in these samples such that they can provide diagnostic power (e.g., antibodies that bind to tau species only present in samples from subject with AD and/or other tauopathies or present in samples from those subjects at elevated and/or distinct levels) have not previously been identified. Without being bound by theory, in some embodiments an antibody or antigen binding fragment disclosed herein provides an improvement over the art because of its ability to bind a phosphorylated epitope on tau that is present in certain types of samples (e.g., blood or CSF) from a patient with AD at a greater level than in a comparable sample from a patient with another tauopathy, other neurologic disease, or in a healthy subject. This difference can be used, in some embodiments, to detect whether a subject presenting with dementia has AD, another tauopathy, or another cause of dementia, and/or to monitor the course of AD treatment. For example, distinct thresholds or fold differences in the amount of tau bound by the antibody (e.g., as compared to levels in healthy subjects or as compared to subjects with known causes of dementia) can be detected and correlated with AD, another tauopathy, or another cause of dementia in a subject. In some embodiments, this difference can be used to detect whether a subject presenting with dementia has AD or another tauopathy. In some embodiments, this difference can be used to detect whether a subject presenting with dementia has AD or some other cause of dementia. In some embodiments, this difference can be used to distinguish whether a subject presenting with dementia has AD or another tauopathy. In some embodiments, this difference can be used to distinguish whether a subject presenting with dementia has AD or some other cause of dementia.

In various embodiment, a method of detecting a tauopathy in a subject is disclosed, comprising: obtaining a biological sample from the subject; contacting the sample from the subject with an effective amount of a molecule that is capable of forming a tau-molecule complex (e.g., at least one receptor, antibody, or antigen binding fragment disclosed hererin that is capable of binding tau to form a tau-antibody complex); detecting the presence and/or amount of the tau-molecule complex using an antibody or antigen binding fragment disclosed herein; wherein the presence and/or amount of tau-molecule complex indicates a tauopathy in the subject. In various embodiments a method of detecting a tauopathy in a subject comprises: contacting a biological sample from the subject with an effective amount of at least one antibody or antigen binding fragment disclosed herein that is capable of binding tau to form a tau-antibody complex, wherein the presence and/or amount of tau-antibody complex indicates a tauopathy in the subject. In some embodiments, detection is by an immunomagnetic reduction bio-assay, e.g., using a bioassay device from MagQu Co. Ltd.

In some embodiments, the tau detected in the sample is a phosphorylated tau. In various embodiments, the at least one antibody or antigen binding fragment can bind an epitope on tau comprising one or more of residues 188-227 of tau protein 2N4R (SEQ ID NO:10). In various embodiments, the at least one antibody or antigen binding fragment can bind an epitope on tau comprising one or more of residues 210-221 of tau protein 2N4R (SEQ ID NO:11). In some embodiments, the antibody or antigen binding fragments used in the methods comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6.

In certain embodiments, the antibody or antigen binding fragment used in the methods comprise a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1 with a substitution at position 2, HCDR2 comprises the amino acid sequence of SEQ ID NO: 2 with a substitution at one or more of position 2 and 12, and/or HCDR3 comprises the amino acid sequence of SEQ ID NO: 3, and/or wherein LCDR1 comprises the amino acid sequence of SEQ ID NO: 4 with a substitution at position 2, LCDR2 comprises the amino acid sequence of SEQ ID NO: 5, and/or LCDR3 comprises the amino acid sequence of SEQ ID NO: 6. In various embodiments, the the substitution at position 2 in HCDR1 is glycine, the substitution at position 2 in HCDR2 is isoleucine, the substitution at position 12 in HCDR2 is valine, and/or the substitution at position 2 in LCDR1 is asparagine.

In various embodiments, the antibody or antigen binding fragment comprises any of the sequences shown in table 1, e.g., a set of six CDRs and or paired heavy and light chain variable domain sequences listed in Table 1.

In certain embodiments, the antibody or antigen binding fragment used in the methods comprise a heavy chain variable region and a light chain variable region, wherein the heavy chain variable region comprises an amino acid sequence of SEQ ID NO: 7 and the light chain variable region comprises an amino acid sequence of SEQ ID NO: 8. The antibody or antigen binding fragment used in the disclosed methods may, in various embodiments, comprise DC2E7 or an antigen binding fragment thereof or any of the variants disclosed herein that are capable of binding to tau. In various embodiments, the antigen or antigen binding fragment further comprises a detectable label.

In some embodiments, the heavy chain variable region comprises the amino acid sequence of SEQ ID NO: 7 or SEQ ID NO: 7 with a substitution at one or more of position 1, 2, 3, 9, 12, 19, 30, 31, 35, 37, 42, 43, 48, 49, 51, 54, 55, 56, 58, 62, 63, 64, 65, 66, 68, 69, 70, 73, 76, 77, 78, 79, 80, 83, 84, 88, 94, 96, 107, 108, and 112, and/or the light chain variable region comprises the amino acid sequence of SEQ ID NO: 8 or SEQ ID NO: 8 with a substitution at one or more of position 3, 7, 11, 14, 17, 19, 20, 21, 24, 25, 28, 39, 42, 49, 52, 56, 69, 71, 75, 92, 94, 99, 105, and 106. In some embodiments, the substitution in the heavy chain variable region at position 1 is glycine, at position 2 is alanine, at position 3 is arginine, at position 9 is arginine, at position 12 is alanine, at position 19 is arginine, at position 30 is glycine, at position 31 is glycine, at position 35 is arginine, at position 37 is alanine, at position 42 is glycine, at position 43 is methionine, at position 48 is isoleucine, at position 49 is threonine, at position 51 is valine, at position 54 alanine, at position 55 is glycine, at position 56 is serine, at position 58 is valine, at position 62 is glycine, at position 63 is alanine, at position 64 is selected from alanine and glutamic acid, at position 65 is glutamic acid, at position 66 is aspartic acid, at position 68 is leucine, at position 69 is alanine, at position 70 is threonine, at position 73 is asparagine, at position 76 is glutamic acid, at position 77 is serine, at position 78 is alanine, at position 79 is methionine, at position 80 is selected from serine, leucine, and histidine, at position 83 is threonine, at position 84 is alanine, at position 88 is proline, at position 94 is cysteine, at position 95 is glycine, at position 107 is alanine, at position 108 is proline, and/or at position 112 is proline, and/or the substitution in the light chain variable region at position 3 is arginine, at position 7 is proline, at position 11 is selected from serine and leucine, at position 14 is proline, at position 17 is valine, at position 19 is alanine, at position 20 is alanine, at position 21 is valine, at position 24 is glutamic acid, at position 25 is threonine, at position 28 asparagine, at position 39 isoleucine, at position 42 is selected from serine and aspartic acid, at position 49 is proline, at position 52 is glycine, at position 56 is proline, at position 69 is glycine, at position 71 is histidine, at position 75 is valine, at position 92 is arginine, at position 94 is threonine, at position 99 is serine, at position 105 is glycine, and/or at position 106 is valine.

In various embodiments, the substitution in the heavy chain variable region at position 68 is leucine, and the substitution in the light chain variable region at position 39 is isoleucine. In various embodiments, the substitution in the heavy chain variable region at position 48 is isoleucine. In various embodiments, the substitution in the heavy chain variable region at position 30 is glycine and at position 58 is valine, and the substitution in the light chain variable region at position 7 is proline and at position 69 is glycine. In various embodiments, the substitution in the light chain variable region at position 11 is serine. In various embodiments, the substitution in the heavy chain variable region at position 77 is serine, and the substitution in the light chain variable region at position 11 is leucine, at position 20 is alanine, and at position 28 is asparagine. In various embodiments, the substitution in the heavy chain variable region at position 37 is alanine, at position 63 is alanine, and position 80 is serine, and the substitution in the light chain variable region at position 11 is leucine. In various embodiments, the substitution in the heavy chain variable region at position 1 is glycine, at position 3 is arginine, at position 76 is glutamic acid, and at position 77 is serine. In various embodiments, the substitution in the light chain variable region at position 11 is leucine.

In various embodiments, the antibody or antigen binding fragment comprises any of the sequences shown in table 1, e.g., a set of six CDRs and or paired heavy and light chain variable domain sequences listed in Table 1.

In some embodiments, the method comprises contacting a biological sample from a subject (e.g., blood or CSF) with at least one capture antibody and at least one detection antibody. In some embodiments, more than one capture antibody and/or more than one detection antibody is used. In some embodiments, the capture and detection antibodies are the same (e.g., when detecting tau oligomers in a sample). In some embodiments, a first antibody or antigen binding fragment (e.g., a detection antibody) capable of forming a tau-antibody complex, and wherein the presence of the tau-antibody complex is detecting using a second anti-tau antibody or antigen binding fragment (e.g., a detection antibody) disclosed herein. In some embodiments, the second antibody or antigen binding fragment binds a different epitope on tau than the first antibody.

In some embodiments, the first antibody or antigen binding fragment can bind an epitope on tau comprising one or more of residues 188-227 of tau protein 2N4R (SEQ ID NO: 10) or one or more of residues 210-221 of tau protein 2N4R (SEQ ID NO: 11). The epitope may comprise at least one phosphorylated residue at position 217 of tau protein 2N4R (SEQ ID NO: 9) and optionally also may comprise a phosphorylated serine at position 210, threonine at position 212, serine at position 214, or threonine at position 220 of tau protein 2N4R, or any combination thereof. In certain embodiments, the epitope on tau comprises or consists of SRTPSLPpTPPTR (SEQ ID NO: 12). In certain embodiments, the epitope on tau comprises or consists of SRpTPSLPpTPPTR (SEQ ID NO 31). In some embodiments, the first antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the first antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the first antibody or antigen binding fragment comprises antibody DC2E7 or an antigen binding fragment thereof.

In another embodiment, the first antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 23, HCDR2 comprises the amino acid sequence of SEQ ID NO: 24, and HCDR3 comprises the amino acid sequence of SEQ ID NO: 25; and wherein LCDR1 comprises the amino acid sequence of SEQ ID NO: 26, LCDR2 comprises the amino acid sequence of SEQ ID NO: 27, and LCDR3 comprises the amino acid sequence of SEQ ID NO: 28. In some embodiments, the heavy chain variable region comprises an amino acid sequence of SEQ ID NO: 29 and the light chain variable region comprises an amino acid sequence of SEQ ID NO: 30.

In various embodiments, the second antibody or antigen binding fragment may bind to an epitope on tau comprising one or more of residues 151-188 of tau protein 2N4R (SEQ ID NO: 13). In some embodiments, the epitope comprises one or more of residues 163-172 of tau protein 2N4R (SEQ ID NO: 14). In certain embodiments, one or more of the residues on the epitope are phosphorylated, e.g., a phosphorylated threonine at position 169 of tau protein 2N4R. In some embodiments, the second antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20. In certain embodiments, the second antibody or antigen binding fragment comprises a heavy chain variable region and a light chain variable region, wherein the heavy chain variable region comprises an amino acid sequence of SEQ ID NO: 21 and the light chain variable region comprises an amino acid sequence of SEQ ID NO: 22. In an embodiment, the second antibody or antigen binding fragment comprises antibody DC2E2 or an antigen binding fragment thereof.

In other embodiments, the first and second antibodies are reversed, and/or more than one first and second antibody are used (which may be the same or different antibodies).

In some of the reversed embodiments, the first antibody or antigen binding fragment may bind to an epitope on tau comprising one or more of residues 151-188 of tau protein 2N4R (SEQ ID NO: 13). In some embodiments, the epitope comprises one or more of residues 163-172 of tau protein 2N4R (SEQ ID NO: 14). In certain embodiments, one or more of the residues on the epitope are phosphorylated, e.g., a phosphorylated threonine at position 169 of tau protein 2N4R. In some embodiments, the first antibody or antigen binding fragment comprises any of the sequences shown in table 1, e.g., a set of six CDRs and or paired heavy and light chain variable domain sequences listed in Table 1.

In some embodiments, the first antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20. In an embodiment, the first antibody or antigen binding fragment comprises a heavy chain variable region comprising an amino acid sequence of SEQ ID NO: 21 and a light chain variable region comprising an amino acid sequence of SEQ ID NO: 22. In an embodiment, the first antibody or antigen binding fragment comprises antibody DC2E2 or an antigen binding fragment thereof.

In some of the reversed embodiments, the second antibody or antigen binding fragment can bind an epitope on tau comprising one or more of residues 188-227 of tau protein 2N4R (SEQ ID NO: 10) or one or more of residues 210-221 of tau protein 2N4R (SEQ ID NO: 11). The epitope bound by the second antibody or antigen binding fragment may comprise at least one phosphorylated residue at position 217 of tau protein 2N4R (SEQ ID NO: 9). In some embodiments, the epitope also comprises a phosphorylated serine at position 210, threonine at position 212, serine at position 214, or threonine at position 220 of tau protein 2N4R, or any combination thereof. In certain embodiments, the second antibody may bind an epitope on tau comprising SRTPSLPpTPPTR (SEQ ID NO: 12). In some embodiments, the second antibody or antigen binding fragment comprises any of the sequences shown in table 1, e.g., a set of six CDRs and/or paired heavy and light chain variable domain sequences listed in Table 1.

In some embodiments, the second antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the second antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the second antibody or antigen binding fragment comprises antibody DC2E7 or an antigen binding fragment thereof.

In various embodiments, the first or second antibody or antigen binding fragment is linked and/or coated onto a solid surface or particle. The solid surface may be the surface of a microtiter plate. A microtiter plate or multiwell plate typically has, e.g., 6, 12, 24, 48, 96, 384, or 1536 or more sample wells arranged in a 2:3 rectangular matrix. Each well typically holds somewhere between tens of nanoliters to several milliliters of liquid. If a solid particle is used in place of or in addition to a solid surface, it may be a bead. The particle may be a magnetic bead. The bead may be a plastic or synthetic polymer bed comprising: polyethylene, polypropylene, polystyrene, polyamide, polyurethane, phenolic polymer, nitrocellulose, naturally derived polymer, latex rubber, polysaccharide, polypeptide, composite material, ceramic, silica or silica-based material, carbon, metal or metal compound, gold, silver, steel, aluminum, copper, inorganic glass, or silica material, or a combination thereof. The bead may have a spherical, disk, ring, or cube-like shape.

In certain embodiments, the first and/or second antibody is conjugated to a detectable label. The label may comprise an enzyme, a radioisotope, biotin, a nuclear magnetic resonance marker, a heavy metal, or a combination thereof. The method may further comprise detecting a signal from the detectable label. In one embodiment, detecting the label is achieved by detection of a fluorescent signal or the intensity of that signal from a labeled antibody following binding to tau. In some embodiments, the detectable label is biotin and it is detected by contacting the sample with streptavidin conjugated to an enzyme, preferably horse radish peroxidase, alkaline phosphatase, or β-galactosidase.

In certain embodiments, the tau detection methods disclosed herein comprises a classic/conventional ELISA assay (i.e., analog readout systems). Additionally or alternatively, the methods may comprise a digital ELISA (i.e., digital readout systems which enable concentrations to be determined digitally rather than by measurement of the total analog signal down to a single immunocomplex, although, at some concentrations, these methods can also be used to read analog signals). See Rissin, D. M., et al., Single-molecule enzyme-linked immunosorbent assay detects serum proteins at subfemtomolar concentrations. Nat. Biotechnol., 2010. 28(6), 555-559. For example, a single-molecule array (simoa) may be used, which is a digital ELISA in which, after the formation of a sandwich complex on magnetic microbeads, the beads are transferred, in substrate solution, to an array of micro wells, e.g., femtoliter-sized micro wells. In some embodiments, these wells accommodate only one bead each. In some embodiments, after the addition of a fluorogenic substrate for the enzyme with which the detection antibody is labeled, an oil film is then applied to seal the wells to a small volume, e.g., confining the reaction volume to 50 fL. In some embodiments, this small volume allows for a readable signal to be detected even if only one sandwich complex is present on the bead. As a reporter, the enzyme β-galactosidase and the substrate resorufin-β-D-galactopyranoside may be used and the wells having a detectable signal are counted as are all the wells containing a bead. The ratio between these counts can provide an average enzyme output per bead (AEB). When the AEB is low (<0.1), Poisson statistics may be used to show that either a bead has only one or less complex on its surface. When the AEB signal gets higher, increasing the probability of more than one complex per bead, a transition to light intensity calculations may be used, which allows for a usable AEB even at signals >0.1. The algorithm for the transition may be implemented using software for a Simoa instrument, e.g., software which samples and calibrates, e.g., from a 96-well microtiter plate or separate vials.

Other immunoassays are known in the art and may be used with the disclosed antibodies and labeled antibodies to detect phosphorylated tau in a sample. For instance, a single-molecule counting (SMC) platform may be used, e.g., one in which antibodies with and without fluorescent labels form sandwich complexes with tau, either on beads or plates, the complexes are then broken up and the molecules of fluorescently labeled (e.g., Alexa Fluor) detection antibody are drawn into a capillary tube and counted as they pass a laser beam that excites the fluorophore. A digital event can be counted if the fluorescence reaches above a background threshold. At higher concentrations, the total sum of all emitted photons may be used as readout for the signal, allowing for a high dynamic range. In some embodiments, detection is by single-molecule counting, e.g., using a single-molecule counting device from Merck Millipore (developed by Singulex). Another high sensitive immunoassay that may be used involves attaching magnetic nanoparticles to an antibody disclosed herein, and detecting an alteration in the oscillation of the nanoparticles in an alternating magnetic field in a concentration-dependent manner after binding to the analyte, e.g., detecting immunomagnetic reduction (IMR). In some embodiments, detection is by an immunomagnetic reduction bio-assay, e.g., using bio-assay from MagQu Co. Ltd.

In various embodiments, a sample may be diluted prior to being contacted with an antibody or antigen binding fragment thereof. In certain situations, circulating antigens can be masked by naturally existing antibodies against those antigens that also circulate in the blood. The HIV protein p24 is a well-known example. In addition, naturally existing tau/anti-tau antibody complexes have been reported in the literature. Wu J, Li L. Autoantibodies in Alzheimer's disease: potential biomarkers, pathogenic roles, and therapeutic implications. Journal of Biomedical Research. 2016; 30(5):361-372. The presence of those complexes may interfere with the detection assays disclosed herein. Accordingly, in various embodiments, the sample may be subjected to immune complex dissociation (ICD) (e.g., dissociating naturally existing tau-antibody/tau-molecule complex in the sample such that tau is no longer bound to the naturally existing antibody/molecule and is thus free to be detected by the methods of the disclosure) prior to being contacted with an antibody or antigen binding fragment of the disclosure. In some embodiments, immune complex dissociation is achieved by applying heat and/or acid to the sample or any other known method of achieving ICD.

In any of the preceding embodiments, the biological sample can comprise cerebrospinal fluid (CSF). CSF can be obtained from a patient, e.g., by a lumbar puncture performed in a medical setting. Alternatively, the biological sample can comprise blood plasma and/or or serum.

In various embodiments, the methods disclosed herein detect the presence and/or amount of a phosphorylated tau in a sample, which is compared to the amount of phosphorylated tau in the sample to the level in a control sample from a healthy individual, and wherein an increase in the level of phosphorylated tau in the sample over the control indicates a tauopathy. In another embodiment, the detected tauopathy is Alzheimer's disease. In certain embodiments, an increase in the level of phosphorylated tau in a sample from a subject over a sample from a healthy control and/or a patient with a known non-AD tauopathy sample indicates the subject has Alzheimer's disease rather than another tauopathy or another cause of dementia.

For instance, a 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 75, 100, or more fold increase in the level of tau detected by an antibody or antigen binding fragment (or any fold increase in between) in a sample from a subject with dementia as compared to the level in a control sample from a healthy subject may indicate the presence of AD or another tauopathy. In some embodiments, the fold increase is between about 1-100 fold, or about 2-3 fold. In some embodiments, the fold increase is between about 1-50, 1-25, 1-10, or 1-5 fold. In some embodiments, a level of bound tau in a sample from a subject with dementia greater than a threshold (e.g., 0.1, 0.2, 0.3, 0.4, 0.5, 0.6, 0.7, 0.8, 0.9, 1, 1.5, 2, 2.5, 3, 4, 5, 6, 7, 8, 9, 9.3 10, 11, 12, 13, 14, 15, 16, 17, 18, 19, 20, 25, 30, 35, 40, 45, 50, 55, 60, 65, 70, 75, 80, 85, 90, 95, 100, 125, 150, 200, 250, 300, 350, 400, 450, 500, 550, 600, 650, 700, 750, 800, 850, 900, 950, or 1000 pg/ml, or any value in between) observed in a control sample from a healthy subject may indicate the presence of AD or another tauopathy. In some embodiments, the threshold is about 9.3 pg/ml of tau. In some embodiments, the threshold is any value between 0.93 and 93 pg/ml of tau. In some embodiments, the threshold is about 5.37 pg/ml. In some embodiments, the threshold is about 305 pg/ml. In some embodiments, the threshold is about 100-600 pg/ml. In some embodiments, a 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, or more fold increase in the level of tau detected by an antibody or antigen binding fragment (or any fold increase in between) in a sample from a subject with dementia as compared to the level in a control sample from a patient with a known tauopathy other than AD (e.g., FTD, CBD, or PSP), or a control sample from a patient with another form of dementia, may indicate the presence of AD in the subject with dementia. In some embodiments, the fold increase is between about 1-100 fold, or about 1-50 fold, or about 1-25 fold, or about 1-5 fold, or about 2-3 fold. In some embodiments, a level of bound tau in a sample from a subject with dementia greater than a threshold (e.g., 0.1, 0.2, 0.3, 0.4, 0.5, 0.6, 0.7, 0.8, 0.9, 1, 1.5, 2, 2.5, 3, 4, 5, 6, 7, 8, 9, 9.3, 10, 11, 12, 13, 14, 15, 16, 17, 18, 19, 20, 25, 30, 35, 40, 45, 50, 55, 60, 65, 70, 75, 80, 85, 90, 95, 100, 125, 150, 200, 205, 210, 215, 220, 225, 230, 235, 240, 245, 250, 255, 260, 265, 270, 275, 280, 285, 290, 300, 305, 310, 315, 320, 325, 330, 335, 340, 345, 350, 355, 360, 365, 370, 375, 380, 385, 390, 395, 400, 410, 420, 430, 440, 450, 460, 470, 480, 490, 500, 510, 520, 530, 540, 550, 560, 570, 580, 590, 600, 650, 700, 750, 800, 850, 900, 950, or 1000 pg/ml, or any value in between) observed in a control sample from a patient with a known tauopathy other than AD (e.g., FTD, CBD, or PSP), or a control sample from a patient with another form of dementia, may indicate the presence of AD in the subject with dementia. In various embodiments, the level of tau detected by antibody binding is quantified using routine methods, e.g., by measuring fluorescence intensity, by Western blot, mass spectrometry, classic ELISA, digital ELISA, or other methods known to the skilled artisan.

In some embodiments, the threshold is about 0.1-10 pg/ml when the detection assay is calibrated using a phosphorylated tau protein, i.e., a full-length tau protein comprising one or more phosphorylated positions. In some embodiments, the threshold is about 100-600 pg/ml when the detection assay is calibrated using a 2E7 synthetic peptide (2E7pep) with the following sequence: GQKGQANATRIPAKGGGSGGGSGGGSSRTPSLPpTPPTREPK.

In various embodiments, a method for distinguishing Alzheimer's disease from another tauopathy or another cause of dementia in a subject is disclosed, comprising: obtaining a biological sample from the subject; contacting the sample from the subject with an effective amount of a molecule that is capable of forming a tau-molecule complex (e.g., at least one receptor, or antibody, or antigen binding fragment disclosed hererin that is capable of binding tau to form a tau-antibody complex); detecting the presence and/or amount of the tau-molecule complex using an antibody or antigen binding fragment disclosed herein; wherein the presence and/or an elevated level of phosphorylated tau bound to the molecule in the sample relative to the level in a sample from a healthy control subject and/or relative to the level in a sample from a subject with a known tauopathy other than AD (e.g., FTD, CBD, or PSP) indicates the subject has Alzheimer's disease rather than another tauopathy or an alternative cause of dementia. In various embodiments, the subject with Frontotemporal dementia (FTD) may have Nonfluent/Agrammatic Primary Progressive Aphasia (nfPPA), Semantic Variant Primary Progressive Aphasia (svPPA), Behavioral Variant Frontotemporal Dementia (bvFTD), or Amyotrophic Lateral Sclerosis/Frontotemporal Dementia (ALS/FTD).

In various embodiments, a method for distinguishing Alzheimer's disease from another tauopathy or another cause of dementia in a subject is disclosed, comprising: obtaining a cerebrospinal fluid or blood sample from a subject; contacting the sample with an anti-tau antibody or antigen binding fragment thereof disclosed herein; and detecting the presence and/or amount of the tau-antibody complex using an antibody or antigen binding fragment disclosed herein, wherein the presence and/or an elevated level of phosphorylated tau bound to the antibody in the sample relative to the level in a sample from a healthy control subject and/or relative to the level in a sample from a subject with a known tauopathy other than AD (e.g., FTD, CBD, or PSP) indicates the subject has Alzheimer's disease rather than another tauopathy or another form of dementia. In some embodiments, the method uses an antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6, wherein the antibody or antigen binding fragment is capable of binding to phosphorylated tau to form a phosphorylated tau-antibody complex; and detecting the presence and/or amount of phosphorylated tau complexed with the antibody or antigen binding fragment in the sample, wherein the presence and/or an elevated level of phosphorylated tau in the sample relative to the level in a sample from a healthy control subject indicates the subject has Alzheimer's disease rather than another tauopathy or an alternative cause of dementia. In certain embodiments, the antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the antibody or binding fragment comprises antibody DC2E7 or an antigen binding fragment thereof.

In various embodiments, the antibody-tau complex is detected with a second antibody or antigen binding fragment thereof capable of binding tau. In some embodiments, the second antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20. In certain embodiments, the second antibody or antigen binding fragment comprises a heavy chain variable region comprising an amino acid sequence of SEQ ID NO: 21 and a light chain variable region comprising an amino acid sequence of SEQ ID NO: 22. In various embodiments, second antibody or antigen binding fragment comprises antibody DC2E2 or an antigen binding fragment thereof. In some embodiments, the detection and capture antibodies are reversed.

In various embodiments, a method of treatment is disclosed, comprising administering a therapeutic agent for Alzheimer's disease to a subject suffering from Alzheimer's disease, wherein the subject has been identified as having Alzheimer's disease according to any of the preceding methods. Treatment may comprise administering an antibody, therapeutic peptide, or small molecule that treats AD, e.g., any of the treatments mentioned herein. In various embodiments, therapeutic agents may include one or more of the antibodies or therapeutic peptides disclosed in U.S. Pat. No. 9,518,101 and/or WO 2016/079597, which are hereby incorporated by reference in their entirety. In certain embodiments, the therapeutic agent may be antibody DC2E7 or an antigen binding fragment thereof and/or DC2E2, or an antigen binding fragment thereof. In an embodiment, the therapeutic agent may be an antibody or an antigen binding fragment thereof that binds to the same epitope as DC2E7 and/or DC2E2.

Also disclosed herein, in certain embodiments, are uses of the disclosed antibodies and antigen binding fragments conjugated to detectable labels (e.g., radiolabels) that can be administered (e.g., intravenously) to a subject to detect the pattern of phosphorylated tau in the brain and thereby diagnose AD or another tauopathy.

In various embodiments, a method of detecting Alzheimer's disease or another tauopathy in a human subject is disclosed, comprising administering to the subject the antibody or antigen binding fragment disclosed herein conjugated to a detectable label such as a radioisotope and detecting a signal from the radioisotope or other detectable label in the brain of the patient, wherein the detection of a signal indicates the subject has Alzheimer's disease or another tauopathy. In some embodiments, the brain distribution of tau species bound by the disclosed antibodies and antigen binding fragments conjugated to detectable labels may be used to deteremine whether a subject has AD or another tauopathy. For instance, antibody binding may differ in AD (where the tau species may be more prominent in hippocampus CA1) as compared to other tauopathies such as FTD-Pick's disease (where the tau species may be more prominent in dentate gyrus, and, to some extent, hippocampus), CBD (where the tau species may be more prominent in nucleus caudatus) and PSP (where the tau species may be more prominent in putamen/nucleus caudatus). In some embodiments, a subject is administered an antibody and antigen binding fragment conjugated to a detectable label (e.g., via intravenous administration) and then their brain is imaged (e.g., via PET) to generate a map of tau bound by the labeled antibody and antigen binding fragment in the brain. The map may be analyzed against known maps for AD and other tauopathies to determine whether the patient has AD or another tauopathy.

In certain embodiments, the detection is done by positron emission tomography. The distribution of the signal in the brain may indicate whether the subject has Alzheimer's disease or another tauopathy. See Murray et al., Clinicopathologic assessment and imaging of tauopathies in neurodegenerative dementias, Alzheimer's Research & Therapy, 6:1, 2014. In certain embodiments, the antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In an embodiment, the heavy chain variable region comprises an amino acid sequence of SEQ ID NO: 7 and the light chain variable region comprises an amino acid sequence of SEQ ID NO: 8. In an embodiment, the antibody or binding fragment comprises antibody DC2E7 or an antigen binding fragment thereof. In an embodiment, the antibody or binding fragment comprises an antibody or antigen binding fragment that competes for binding or binds the same epitope as antibody DC2E7 or an antigen binding fragment thereof.

In various embodiments, an antibody or antigen binding fragment thereof conjugated to a radioisotope may be administered to a subject as described herein. The radiosignal may be detected in the brain by positron emission tomography. Three dimensional images of the antibody conjugate concentration within the body may then be constructed by computer analysis. The concentrations of signal throughout the brain can be used to correlate with distinct patterns associated with AD or other tauopathies, or to determine whether a patient presenting with dementia has or does not have AD, another tauopathy, or another cause of their dementia. These signal patterns may be interpreted by one skilled in the art to determine if a subject has AD or another tauopathy. See Murray et al., Clinicopathologic assessment and imaging of tauopathies in neurodegenerative dementias, Alzheimer's Research & Therapy, 6:1, 2014.

In various embodiments, a human subject presenting with symptoms of dementia is first subjected to any one or more of the methods of detecting Alzheimer's disease or another tauopathy disclosed herein before a treatment decision is made. In certain embodiments, a sample (e.g., CSF or blood) is taken from a human subject presenting with symptoms of dementia and analyzed according to the methods described above, and/or the human subject is administered antibody DC2E7 conjugated with a radioisotope and a signal is detected in the subject's brain to determine if the subject has Alzheimer's disease or another tauopathy. In an embodiment, a human subject who has been determined to have Alzheimer's disease or another tauopathy may be administered a pharmaceutical composition that treats AD or another tauopathy. In an embodiment, the pharmaceutical composition comprises an antibody or antigen binding fragment disclosed herein. In an embodiment, the pharmaceutical composition comprises one or more of the antibodies or therapeutic peptides disclosed in U.S. Pat. No. 9,518,101 and/or WO 2016/079597. Alternatively, if the subject is determined not to have AD or another tauopathy, an alternative treatment may be administered.

In various embodiments, a method of determining the stage of Alzheimer's disease in a human subject is disclosed, comprising: obtaining a cerebrospinal fluid or blood sample from the subject, contacting the sample with an effective amount of a molecule that is capable of forming a tau-molecule complex (e.g., at least one receptor, antibody, or antigen binding fragment disclosed hererin that is capable of binding tau to form a tau-antibody complex); detecting the amount of the tau-molecule complex using an antibody or antigen binding fragment disclosed herein; and comparing the amount of tau complexed with the molecule to the amount in a sample of known AD stage or a threshold, thereby identifying the stage of Alzheimer's disease. In some embodiments, a higher amount of tau in the sample indicates a more advanced stage of AD. In various embodiments, levels and/or thresholds are evaluated as described above. In some embodiments, an advanced stage is determined by comparison to the amount of phosphorylated tau detected in a sample of known AD stage, e.g., Braak Stages I-VI. Braak et al., “Neuropathological stageing of Alzheimer-related changes”. Acta Neuropathologica, 82(4): 239-59 (1991).

In various embodiments, a method of determining the stage of Alzheimer's disease in a human subject is disclosed, comprising: obtaining a cerebrospinal fluid or blood sample from the subject, contacting the sample with an antibody or antigen binding fragment disclosed herein, wherein the antibody or antigen binding fragment is capable of binding to phosphorylated tau to form a phosphorylated tau-antibody complex, detecting the amount of phosphorylated tau complexed with the antibody or antigen binding fragment in the sample, and comparing the amount of tau complexed with the antibody to the amount in a sample of known AD stage or a threshold, thereby identifying the stage of Alzheimer's disease. In some embodiments, a higher amount of tau in the sample indicates a more advanced stage of AD. In various embodiments, levels and/or thresholds are evaluated as described above. In some embodiments, an advanced stage is determined by comparison to the amount of phosphorylated tau detected in a sample of known AD stage, e.g., Braak Stages I-VI. Braak et al., “Neuropathological stageing of Alzheimer-related changes”, Acta Neuropathologica, 82(4): 239-59 (1991). In some embodiments, the antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the antibody or binding fragment comprises antibody DC2E7 or an antigen binding fragment thereof. In some embodiments, the level of tau determined in the sample is compared to the level in a sample from a patient of known AD stage and/or to a control sample from a healthy individual to determine whether the test subject has an elevated amount of tau in their sample.

In various embodiments, a method of determining the effectiveness of an anti-tau therapy for Alzheimer's disease is disclosed, comprising: obtaining a cerebrospinal fluid or blood sample from a human subject; contacting the sample with an antibody or antigen binding fragment disclosed herein, wherein the antibody or antigen binding fragment is capable of binding to phosphorylated tau to form a phosphorylated tau-antibody complex; and detecting the presence and/or amount of phosphorylated tau complexed with the antibody or antigen binding fragment in the sample, wherein an elevated level of phosphorylated tau in the sample relative to the level in a sample from a healthy control subject or a threshold indicates the subject is more likely to respond to an anti-tau therapy for Alzheimer's disease. In various embodiments, an elevated level over a threshold or a fold increase in tau is determined as described above.

In some embodiments, the anti-tau antibody or antigen binding fragment used in determining the effectiveness of an anti-tau therapy for Alzheimer's disease comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the antibody or binding fragment comprises antibody DC2E7 or an antigen binding fragment thereof.

In certain embodiments, an anti-tau therapy is administered to a subject identified as being more likely to respond to the therapy. In an embodiment, the anti-tau therapy comprises administering an antibody that binds to tau and promotes its clearance from the brain or inhibit the spreading of tau pathology. In an embodiment, the antibody or antigen binding fragment comprises comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In an embodiment, the anti-tau therapy comprises any of those disclosed herein, and/or any disclosed in U.S. Pat. No. 9,518,101 and WO 2016/079597. In various embodiments, the anti-tau therapy comprises a small molecule or peptide vaccine therapy or antibody therapy. See U.S. Pat. No. 9,518,101 and WO 2016/079597, which are incorporated by reference in their entirety.

In various embodiments, a method of monitoring the effectiveness of an anti-tau therapy for Alzheimer's disease is disclosed, comprising: (a) obtaining cerebrospinal fluid or blood sample from a human subject prior to treatment; (b) contacting the sample with an antibody or antigen binding fragment disclosed herein, wherein the antibody or antigen binding fragment is capable of binding to phosphorylated tau to form a phosphorylated tau-antibody complex; (c) detecting the presence and/or amount of phosphorylated tau complexed with the antibody or antigen binding fragment; (d) administering an anti-tau therapy to the subject; (e) repeating steps (a)-(c) after administering the anti-tau therapy, whereby a reduction in the level of phosphorylated tau in the sample after treatment as compared to the level in the sample before treatment indicates an effective therapy. In some embodiments, the anti-tau antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the method further comprises administering the anti-tau therapy again to a subject who has a lower level of phosphorylated tau a the sample obtained after treatment as compared to the level in the sample obtained before treatment. In an embodiment, the antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the antibody or binding fragment comprises antibody DC2E7 or an antigen binding fragment thereof. In various embodiments, the anti-tau therapy comprises a small molecule or peptide vaccine or antibody therapy. See WO 2016/079597 and U.S. Pat. No. 9,518,101, which are incorporated by reference in their entirety. In an embodiment, the anti-tau therapy comprises administering an antibody that binds to tau and promotes its clearance from the brain. In certain embodiments, the anti-tau therapy comprises administering an anti-tau antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In an embodiment, the anti-tau therapy comprises any of those disclosed herein, and/or any disclosed in U.S. Pat. No. 9,518,101 and WO 2016/079597.

Other Methods of Monitoring and/or Confirming AD

A number of additional measures may be used to determine, confirm, and/or monitor AD status, e.g., by behavioral assessment, for use in confirming an AD diagnosis or to evaluate the effects of treatment and/or disease progression. In some embodiments, the measures include mean change in Alzheimer's Disease Activity Scale-Cognitive subscale 13 (ADAS-Cog 13) scores and Mean change in Alzheimer's Disease Cooperative Study-Activities of Daily Living (ADCS-ADL) scores. Other measures may include change in MRI volumetry, change in Clinical Dementia Rating (CDR-SB/CDR-GS), change in neuropsychiatric behavior: neuropsychiatric inventory (NPI) total and domain scores, and/or change in cognition: MMSE total score. In another embodiment, the measures can include any of the following: time to the occurrence of death, institutionalization, loss of ability to perform activities of daily living, time to severe dementia, ADCS-ADL, ADAS-cog score, MMSE scores, cognitive performance, plasma CSF biomarkers, ADAS-total score, Quality of life assessed by Quality of Life Alzheimer's disease scale, behavioral test scores, and the US FDA's Clinical Dementia Rating-sum of boxes.

In various embodiments, a method of detecting Alzheimer's diseases or another tauopathy in a human subject comprises administering to a subject an antibody or antigen bringing fragment disclosed herein that has been conjugated to a radioisotope and detecting a signal in the brain of the patient, wherein the detected signal pattern in the brain indicates whether the subject has Alzheimer's disease or another tauopathy. Detection of the signal may be done by positron emission topography. The distribution of the signal in the brain may be used to indicate whether the subject has Alzheimer's disease or another tauopathy. In an embodiment, the administered antibody or antigen binding fragment conjugated to a radioisotope comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In an embodiment, the antibody or antigen binding fragment comprises a heavy chain variable region comprising an amino acid sequence of SEQ ID NO: 7 and a light chain variable region comprising an amino acid sequence of SEQ ID NO: 8. In an embodiment, the antibody or antigen binding fragment comprises DC2E7 or an antigen binding fragment thereof.

The antibodies and antigen binding fragments disclosed herein for therapeutic or diagnostic uses can be administered by any suitable administration route, e.g, parenteral, topical, intradermal, intravenous, oral, subcutaneous, intraperitoneal, intranasal or intramuscular routes. A typical route of administration may be subcutaneous although others may be equally effective. Another typical route may be intramuscular injection. This type of injection is typically performed in the arm or leg muscles. Intravenous injections as well as intraperitoneal injections, intra-arterial, intracranial, or intradermal injections may also be used. The antibodies or antigen binding fragments disclosed herein may be administered as injectable dosages of a solution or suspension of the substance in a physiologically acceptable carrier and/or diluent, e.g., a sterile liquid such as water, oil, saline, glycerol, or ethanol. The antibodies or antigen binding fragments disclosed herein may also be administered by drilling a small hole in the skull for administration, which may allow crossing of the blood brain barrier.

### Kits

In various embodiments, kits are disclosed herein, comprising one or more of the antibodies or antigen binding fragments described herein. In certain embodiments, the kit comprises an antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6. In certain embodiments, the antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8. In an embodiment, the antibody or binding fragment comprises antibody DC2E7 or an antigen binding fragment thereof.

In certain embodiments, the kit comprises an antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3) selected from those in Table 1, e.g., a set of six CDRs from those in table 1. In some embodiments, the antibody or antigen binding fragment comprises a light chain variable domain and a heavy chain variable domain and a light chain variable domain selected from those in Table 1, e.g., paired heavy and light chain variable domain sequences selected from those in Table 1.

In certain embodiments, the kit comprises an antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1 with a substitution at position 2, HCDR2 comprises the amino acid sequence of SEQ ID NO: 2 with a substitution at one or more of position 2 and 12, and/or HCDR3 comprises the amino acid sequence of SEQ ID NO: 3, and/or wherein LCDR1 comprises the amino acid sequence of SEQ ID NO: 4 with a substitution at position 2, LCDR2 comprises the amino acid sequence of SEQ ID NO: 5, and/or LCDR3 comprises the amino acid sequence of SEQ ID NO: 6. In various embodiments, the substitution at position 2 in HCDR1 is glycine, the substitution at position 2 in HCDR2 is isoleucine, and/or the substitution at position 12 in HCDR2 is valine, and/or the substitution at position 2 in LCDR1 is asparagine. In various embodiments, the heavy chain variable region comprises the amino acid sequence of SEQ ID NO: 7 with a substitution at one or more of position 1, 3, 30, 37, 48, 58, 63, 68, 76, 77, and 80, and/or the light chain variable region comprises the amino acid sequence of SEQ ID SEQ ID NO: 8 with a substitution at one or more of position 7, 11, 20, 28, 39, and 69. In various embodiments, the substitution in the heavy chain variable region at position 68 is leucine, and the substitution in the light chain variable region at position 39 is isoleucine. In various embodiments, the substitution in the heavy chain variable region at position 48 is isoleucine. In various embodiments, the substitution in the heavy chain variable region at position 30 is glycine and at position 58 is valine, and the substitution in the light chain variable region at position 7 is proline and at position 69 is glycine. In various embodiments, the substitution in the light chain variable region at position 11 is serine. In various embodiments, the substitution in the heavy chain variable region at position 77 is serine, and the substitution in the light chain variable region at position 11 is leucine, at position 20 is alanine, and at position 28 is asparagine. In various embodiments, the substitution in the heavy chain variable region at position 37 is alanine, at position 63 is alanine, and position 80 is serine, and the substitution in the light chain variable region at position 11 is leucine. In various embodiments, the substitution in the heavy chain variable region at position 1 is glycine, at position 3 is arginine, at position 76 is glutamic acid, and at position 77 is serine. In various embodiments, the substitution in the light chain variable region at position 11 is leucine.

In various embodiments, a kit comprises two or more antibody or antigen binding fragments. In certain embodiments, a kit comprises:

a. an antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 1, HCDR2 comprises the amino acid sequence of SEQ ID NO: 2, and HCDR3 comprises the amino acid sequence of SEQ ID NO: 3; and wherein LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; and
b. another antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20.

In certain embodiments, the kit comprises two or more antibodies or antigen binding fragments, wherein an antibody or antigen binding fragment comprises a heavy chain variable region and a light chain variable region, wherein the heavy chain variable region comprises an amino acid sequence of SEQ ID NO: 7 and the light chain variable region comprises an amino acid sequence of SEQ ID NO: 8, and the other antibody or antigen binding fragment comprises a heavy chain variable region and a light chain variable region, wherein the heavy chain variable region comprises an amino acid sequence of SEQ ID NO: 21 and the light chain variable region comprises an amino acid sequence of SEQ ID NO: 22. In certain embodiments, the kit comprises antibody or DC2E7 or an antigen binding fragment thereof and/or antibody or DC2E2 or an antigen binding fragment thereof.

In various embodiments, a kit comprises two or more antibody or antigen binding fragments. In certain embodiments, a kit comprises:
a. an antibody or antigen binding fragment comprising any of the sequences shown in table 1, e.g., a set of six CDRs and or paired heavy and light chain variable domain sequences listed in Table 1;
b. another antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20.

In various embodiments, a kit comprises two or more antibody or antigen binding fragments. In certain embodiments, a kit comprises:
a. an antibody or antigen binding fragment comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 23, HCDR2 comprises the amino acid sequence of SEQ ID NO: 24, and HCDR3 comprises the amino acid sequence of SEQ ID NO: 25; and wherein LCDR1 comprises the amino acid sequence of SEQ ID NO: 26, LCDR2 comprises the amino acid sequence of SEQ ID NO: 27, and LCDR3 comprises the amino acid sequence of SEQ ID NO: 28;
b. and another antibody or antigen binding fragment comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, and HCDR3 comprises the amino acid sequence of SEQ ID NO: 17; and wherein LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20.

In another embodiment, any of the preceding kits may further comprise instructions for using the one or more antibodies or antigen binding fragments to detect phosphorylated tau in a sample from a subject according to the methods disclosed herein and thereby detect Alzheimer's disease in the subject. The instructions may call for the sample to be cerebrospinal fluid or blood. The kits may further comprise additional components for use with a classic ELISA or a digital ELISA.

### EXAMPLES

Several of the foregoing embodiments are illustrated in the non-limiting examples set forth below. However, other embodiments of the disclosure will be apparent to those skilled in the art from consideration of the specification as a whole. It is intended that the specification and examples be considered as exemplary only. In addition, all references cited herein are to be considered incorporated by reference in their entirety.

### Example 1

### Preparation of Recombinant Human Tau Proteins

Human full-length tau 2N4R and tau deletion mutants: Recombinant tau proteins were generated from clone τ40 (Goedert et al., Multiple isoforms of human microtubule-associated protein tau: sequences and localization in neurofibrillary tangles of Alzheimer's disease, Neuron 3, 519-526 (1989), which was subcloned into the expression plasmid pET-17b (Novagen) and expressed in bacteria. Each tau deletion mutant was verified by DNA-sequencing. All tau deletion mutants and tau peptides were numbered according to the longest human tau isoform 2N4R, which is 441 amino acids in length and thus is also called tau441 (D'Souza, I., and Schellenberg, G. D. (2005). Regulation of tau isoform expression and dementia. Biochimica et biophysica acta 1739, 104-115). Production of tau proteins involved the following steps: a) expression of tau in bacteria; b) tau purification by ion exchange chromatography; c) tau purification by gel-filtration; d) concentration and storage of isolated tau.

a) Bacterial expression of human full-length tau (2N4R) and recombinant tau deletion mutants: human tau (above) expression plasmids were transformed into Escherichia coli (E. coli), production strain BL21(DE3). Bacterial cells containing the appropriate expression plasmid were cultivated and induced as described in “Molecular Cloning: A Laboratory Manual” by Sambrook and Russell (2001). A single colony of BL21(DE3) bacteria, transformed with pET-17b plasmid driving expression of a tau protein or its fragment, were grown at 37° C. in 500 mL of Luria broth medium with 100 pg/ml ampicillin at 300 rpm and induced by the addition of isopropyl-β-D-1-thiogalactopyranoside (IPTG) to a final concentration of 0.4 mM. After further incubation at 37° C. for 3 hours, bacteria were collected by centrifugation at 3,000×g for 15 min at 4° C.

b) Cation-exchange chromatography purifications of the basic and neutral tau proteins (six tau isoforms, point mutants of tau isoform 2N4R: Ser198Ala, Ser199Ala, Ser202Ala, Thr205Ala, Ser208Ala, Ser210Ala, Thr212Ala, Ser214Ala, Thr217Ala, Ser231Ala, tau221-441, tau99-441, tau188-44, tau31-441, tau151-391, tau127-441, tau2N4RΔ(134-168), tau2N4RΔ(49-243) were done essentially as previously described (Krajciova et al., Preserving free thios of intrinsically disordered tau protein without use of a reducing agent, Analytical Biochemistry, 383:343-345, 2008). After expression, the bacterial pellets were resuspended in 10 ml of lysis buffer (50 mM 1,4-piperazinediethanesulfonic acid (PIPES) pH 6.9, 50 mM sodium chloride (NaCl), 1 mM ethylenediaminetetraacetic acid (EDTA), 5 mM dithiothreitol (DTT), 0.1 mM phenylmethylsulfonyl fluoride (PMSF), 5% (v/v) glycerol), quickly frozen in liquid nitrogen, and stored at −80° C. until used for purification of tau proteins. For tau protein purification, the frozen bacterial suspensions were quickly thawed and placed on ice. Bacterial cell walls were broken by sonication on ice by using Sonopuls HD 2200, tip TT-13 (Bandelin, Germany) set to 50% duty cycle, 50 W power output, 6 times for 30 s with 30 s pauses. The lysates were clarified by centrifugation (21,000×g for 15 min at 4° C.) and the supernatants were filtered through a 0.45 μm membrane filter. Large-scale purification of the recombinant tau proteins was done at 6° C. using an AKTA-FPLC workstation (Amersham Biosciences, Sweden). The filtered lysates were loaded at a 3 ml/min flow rate onto a 5-ml HiTrap SP HP column (GE Healthcare, Uppsala, Sweden) equilibrated with the lysis buffer, and washed extensively with 60 ml of the lysis buffer until the baseline at 280 nm became stable. Bound tau proteins were eluted by a gradient (0-30% within 15 ml) of Buffer B (lysis buffer supplemented with 1 M NaCl). Individual 1 mL fractions were collected and analyzed by sodium dodecyl sulfate-polyacrylamide gel electrophoresis (SDS-PAGE). To remove nucleic acids, which copurify with positively charged tau proteins, the fractions containing tau protein were pooled and purified by a second cation-exchange chromatography step, using a 5-ml HiTrap SP HP column (GE Healthcare, Uppsala, Sweden) with a less steep gradient of Buffer B (0-30% in 45 ml). Anion-exchange chromatography purification of the acidic tau proteins (taul-226, taul-136, taul-242) was done as previously described (Csokova et. al, Rapid purification of truncated tau proteins: model approach to purification of functionally active fragments of disordered proteins, implication for neurodegenerative diseases, Protein Expression and Purification, 35:366-372, 2004). After expression, bacterial pellets were resuspended in 10 ml of histidine lysis buffer (20 mM histidine, pH 6.0, 50 mM NaCl, 1 mM EDTA, 5 mM DTT, 0.1 mM PMSF, and 5% (v/v) glycerol). Bacterial cell walls were broken by sonication on ice by using Sonopuls HD 2200, tip TT-13 (Bandelin, Germany) set to 50% duty cycle, 50 W power output, 6 times for 30 s with 30 s pauses. The lysates were clarified by centrifugation (21,000×g for 15 min at 4° C.). Bacterial lysates were precipitated by 1% streptomycin sulfate (Medexport, Russia), incubated on ice for 5 min, clarified by centrifugation (21,000×g for 15 min at 4° C.), and filtered through a 0.45 μm membrane filter. The filtered streptomycin precipitated lysates were loaded at 3 ml/min flow rate onto a 5 ml HiTrap QSepharose HP column (Amersham Biosciences, Sweden) and washed extensively with 30-50 ml histidine lysis buffer until the A280 baseline became stable. Tau proteins were eluted with a two-step salt gradient (0.05-0.5M NaCl in 40 ml followed by 0.5-1 M NaCl in 20 ml) in histidine lysis buffer.

c) In the final gel-filtration step of purification (the same for all tau proteins), pooled tau protein fractions obtained by ion exchange chromatography, were injected onto a gel-filtration column (HiLoad 26/60 Superdex 200 prep grade column, GE Healthcare) at 3 ml/min in either PIPES or Histidine lysis buffer for basic/neutral or acidic tau proteins, respectively, supplemented with 100 mM NaCl. Eluted tau proteins were pooled.

d) For tau protein concentration after gel-filtration purification, pooled fractions were diluted with 1.5 volumes of 2.5% glycerol, and loaded again on a HiTrap SP HP column (basic and neutral tau proteins) or on a HiTrap Q HP column (acidic tau proteins). The concentrated recombinant tau protein was then eluted from the column with a 1 M NaCl step gradient. Finally, the buffer was exchanged to phosphate-buffered saline (PBS, 8.09 mM disodium phosphate (Na2HPO4), 1.47 mM potassium dihydrogen phosphate (KH2PO4), 136.89 mM NaCl, 2.7 mM potassium chloride (KCl)) saturated with argon, using a 5 mL HiTrap Desalting column (GE Healthcare). Protein quantitation of purified samples was done using bicinchoninic acid (BCA) quantitation kits (Pierce, USA), with bovine serum albumin (BSA) as a standard. Tau proteins were aliquoted into working aliquots, snap-frozen in liquid nitrogen, and stored at −70° C.

### Example 2

### Preparation of Hybridoma Cell Lines Producing Monoclonal Antibodies Against Human Tau1-242, Screening of Monoclonal Antibodies by ELISA, and Initial Characterization of Monoclonal Antibody DC2E2

Six-week-old Balb/c mice were primed subcutaneously with 50 μg of recombinant taul-242-(prepared as described in Example 1) in complete Freund's adjuvant (SIGMA) and boosted three times at four-week intervals with 50 μg of the same antigen in incomplete Freund's adjuvant. Three days before the fusion, mice were injected intravenously with 50 μg of the same antigen in PBS. Spleen cells from immunized mice were fused with NS/0 myeloma cells according to the method of Kontsekova et al., The effect of postfusion cell density on establishment of hybridomas, Folia Biol. 34, 18-22 (1988). Splenocytes were mixed with NS/0 myeloma cells (ratio 5:1) and fused for 1 minute in 1 ml of 50% polyethylene glycol (PEG) 1550 (Serve) in serum free Dulbecco's modified Eagle's medium (DMEM) supplemented with 10% dimethyl sulphoxide. The fused cells were resuspended in DMEM containing 20% horse serum, L-glutamine (2 mM), hypoxanthine (0.1 mM), aminopterin (0.04 mM), thymidine (0.016 mM), and gentamycin (40 U/ml), at a density of 2.5×10⁵ spleen cells per well on 96-well plates.

The cells were incubated for 10 days at 37° C. and growing hybridomas were screened for the production of anti-tau1-242-specific monoclonal antibodies by an enzyme-linked immunosorbent assay (ELISA). Microtiter plates were coated overnight with taul-242 (5 pg/ml, 50 μl/well) at 37° C. in PBS. The plates were blocked with 1% nonfat dried milk to reduce nonspecific binding, washed with PBS-0.05% Tween 20, and incubated with 50 μl/well of hybridoma culture supernatant for 1 hr at 37° C. Bound monoclonal antibodies were detected with sheep anti-mouse immunoglobulin (Ig) conjugated with horse radish peroxidase (HRP, DAKO). The reaction was developed with TMB one (Kem-En-Tec Diagnostics) as a peroxidase substrate and stopped with 50 μl of 2 M H₂SO₄. Absorbance at 450 nm was measured using a Powerwave HT (Bio-Tek). Readouts with an absorbance value of at least twice the value of the negative controls (PBS) were considered positive. Positive hybridoma cultures were further subcloned in soft agar according to the procedure described in Kontsekova et al., One-step method for establishing 8-azaguanine-resistant hybridomas suitable for preparation of triomas, J. Immunol. Methods, 145, 247-250 (1991).

The monoclonal antibody DC2E2 (produced by the mouse hybridoma cell line deposited with the American Type Culture Collection, with the ATCC Patent Deposit Designation PTA-124991.) was identified among the positive hybridoma cultures so produced and selected. DC2E2 was further characterized as described below. The antibody isotype was determined to be murine IgG1 by ELISA (FIG. 1) using a mouse Ig isotyping kit (ISO-2, SIGMA).

### Example 3

### Mapping of the DC2E2 Epitope Using Recombinant Tau Deletion Mutants, Tau-Derived Peptides and Mass Spectrometry

Deletion mutants of human tau protein 2N4R were used for epitope mapping of DC2E2 using ELISA (FIG. 2A). Recombinant human tau isoforms 2N4R, 1N4R, 2N3R, 0N4R, 1N3R, 0N3R and tau deletion mutants were prepared as described in Example 1. Microtiter plates were coated overnight at 37° C. with recombinant tau proteins (5 pg/ml in PBS, 50 μl/well). The plates were blocked with PBS-Tween20 (0.1% v/v) to reduce nonspecific binding and were incubated with 50 μl/well of DC2E2 hybridoma culture supernatant, for 1 hr at 37° C. Bound monoclonal antibody was detected with sheep anti-mouse Ig HRP-conjugated (DAKO). The reaction was developed with TMB one solution (Kem-En-Tec Diagnostics) as a peroxidase substrate and stopped with 50 μl of 2M H₂SO₄. Absorbance was measured at 450 nm using a Powerwave HT (Bio-Tek). Readouts with an absorbance value of at least twice the value of the negative controls (PBS) were considered positive. DC2E2 recognized the following human tau proteins: six human tau isoforms (FIG. 2B), tau 1-242, tau 31-441, tau 99-441, tau 1-226, tau151-391, tau 127-441, but failed to recognize the deletion mutants tau 1-136, tau 221-441 and tau 2N4R(A134-168) (FIG. 2C). Together, these findings suggest that DC2E2 recognizes binding sites or epitopes on tau, located in the proline reach region of tau between amino acids 151-188.

To further define the epitope, competition ELISA was performed. For competition experiments, the DC2E2 monoclonal antibody (mAb) was purified from serum-free hybridoma supernatant on a Protein G affinity column, as follows. The hybridoma supernatant was adjusted to pH 7.5, the solution was precleared by centrifugation, filtered through a 0.45 μm membrane filter, and loaded onto a 5 ml Protein G Sepharose column. DC2E2 mAb was eluted from the column with 0.1 M Glycine-HCl, pH 2.7. Eluted fractions were immediately neutralized with 1M Tris-HCl pH 9.0. Pooled fractions were dialyzed against PBS, concentrated by ultrafiltration, and stored at −70° C. The concentration of the antibody was determined by measuring absorbance at 280 nm, using the formula c (mg/ml)=A280 nm/1.43.

For competition ELISA, peptides (tau 141-170, 161-190, 181-210, 171-200) were synthesized by EZBiolabs with purity higher than 90%. ELISA plates (Nunc Medisorp, Thermo Scientific, Denmark) were coated overnight at 4° C. with 50 μl/well of 0.4 pg/ml of recombinant purified tau 1-242 in PBS. The plates were washed 4 times with PBS/Tween 20 (0.1% v/v), and blocked with PBS/Tween 20 for 2 h at 25° C. Peptides were separately dissolved in PBS at a final concentration of 5 mM. Serial dilutions (2,5-fold) of the peptides in PBS/Tween 20 were prepared in polypropylene plates with conical well bottom (Greiner, #651201) (concentration range 200 μM, 32 μM, 12.8 μM, 5.1 μM, 2 μM, 0.8 μM, and 0.3 μM). 60 μl of each dilution were added per well. Purified DC2E2 was diluted to a concentration of 0.6 μg/ml in PBS/Tween 20 and 60 μl of this diluted antibody was mixed with each serial dilution of peptides resulting in 120 μl mixtures with 0.36 ng of antibody/60μl containing each respective test peptide at a concentration of 200 μM, 32 μM, 12.8 μM, 5.1 μM, 2 μM, 0.8 μM, and 0.3 μM. The antibody/peptide mixtures were incubated for 1 hr at 25° C. on a rotating platform set to 250 rpm. Fifty microliters (50 μl) of antibody/peptide mixtures were transferred from the polypropylene plates into taul-242-coated and PBS/Tween 20-blocked plates (in duplicates) and incubated for 1 hr at 25° C. on a rotating platform set to 250 rpm. The plates were washed 4× times with PBS/Tween 20 and incubated for 1 hr at 25° C. on a rotating platform (set to 250 rpm) with 50 μl of Polyclonal Goat Anti-Mouse Immunoglobulins/HRP (Dako, #P0447) diluted 1:1000 in PBS/Tween 20. The plates were washed 4× times with PBS/Tween and then incubated with 50 μl/well of TMB one solution (Kem-En-Tec Diagnostics) as a peroxidase substrate. The reaction was stopped by adding 50 μl of 2 M H₂SO₄ (Merck). Absorbance was measured at 450 nm using a Powerwave HT (Bio-Tek).

Competition ELISA was performed with the following peptides: tau 141-170, 161-190, 171-200, 181-210. (FIG. 2D) Only peptide tau 161-190 competed with tau 1-242 for the binding to DC2E2 (FIG. 2D). However, shifting of peptides to the C-terminus of tau (171-200, 181-210) or to the N-terminus of tau (141-170) led to loss of competing activity with tau 1-242 for binding to DC2E2. These results suggest that the epitope is located between amino acids 161-181.

An LC/MALDI mass spectrometry approach was used with the aim to define the epitope more precisely. Sequence of binding site was identified by binding of proteolytically digested tau proteins to DC2E2 monoclonal antibody immobilized on magnetic beads and subsequent identification of eluted peptides by LC/MALDI mass spectrometry. Tau proteins (tau 2N4R and tau151-391) were digested with the mixture of trypsin, Glu-C, chymotrypsin at 37° C. overnight or formic acid for 2 hours at 108° C. Binding reaction was performed in 1% CHAPS in PBS for 2 hours. Non-bound peptides were removed by three washes with 1% CHAPS in PBS. Bound peptides were eluted by three washes with formic acid and lyophilized. Peptides were separated by UHPLC (Dionex, Ultimate 3000 nano-LC system), fractions were mixed with HCCA matrix solution, and dispensed onto MTP Anchor Chip 384 MALDI sample plate (Bruker Daltonics). The fractionated samples were analyzed with a MALDI TOF/TOF (Ultraflextreme, Bruker Daltonics) instrument operated in the positive ion mode. The collected MS and MS/MS spectra were searched against database of tau proteins using Mascot search engine (Matrix Science).

Database analysis revealed several peptide sequences, all of them containing KGQANATRIP. This peptide is located on tau 2N4R in the region of 163-172, a proline rich region.

### Example 4

### DC2E2 Recognizes an A68 Triplet Specific For Insoluble Tau In Alzheimer's Disease, Phosphorylated Tau, and Unphosphorylated Tau

In Alzheimer's disease, tau is hyperphosphorylated. Therefore, for detail characterization of binding properties of DC2E2, hyperphosphorylated PHF-tau and in vitro phosphorylated tau were examined.

Sarcosyl insoluble tau complexes (PHF-Tau) were isolated from human AD brains using the sarkosyl method (Greenberg and Davies, A preparation of Alzheimer's paried helical filaments that displays distint tau proteins by polyacrylamide gel electrophoresis, PNAS, 87:5827-31, 1990). For protein extraction, frozen human AD brain tissue (frontal cortex, samples of Braak stage VI obtained from the Netherlands brain bank) was homogenized in 10 volumes of cold extraction buffer (10 mM Tris pH 7.4, 0.8 M NaCl, 1 mM EGTA, and 10% sucrose) and homogenate was centrifuged for 20 min at 20,000×g. To prepare sarcosyl-insoluble tau, supernatant was supplemented with N-lauroylsarcosine (SIGMA) to a final concentration of 1% and incubated for 1 h at room temperature, while shaking. After centrifugation at 100,000×g for 1 h, the resulting supernatant was discarded, and a pellet comprising the sarkosyl-insoluble tau fraction was resuspended in 1/50 volume of the supernatant used for the preparation of the insoluble tau.

For in vitro phosphorylation of tau 2N4R and tau151-391, kinase extract was used. Adult rat brain (1 g/2.5 ml) was homogenized in kinase buffer (10 mM TRIS-HCl, pH 7.4; 5 mM EGTA, 2 mM DTT; 1 mM PMSF; 2 mM MgCl₂, leupeptin (20 pg/ml), pepstatin (20 pg/ml), aprotinin (20 pg/ml)) and after centrifugation at 100,000×g for 30 min at 4° C. the supernatant (kinase extract) was used for the phosphorylation of tau. The phosphorylation reaction was carried out at 37° C. in 10 mM TRIS-HCl, pH 7.4; 5 mM EGTA, 2 mM DTT; 1 mM PMSF; leupeptin (20 μg/ml); pepstatin (20 μg/ml); aprotinin (20μg/ml); 2 mM ATP; 2 mM MgCl₂ and 10 μM okadaic acid. The brain kinase extract (5μl) was added to 50 μl tau solution (1 μM) and reaction was incubated at 37° C. for 24 h. The phosphorylation of tau was analysed by SDS-PAGE and immunoblotting using rabbit anti-serum raised against six recombinant human tau isoforms (Csokova et al., Rapid purification of truncated tau proteins: model approach to purification of functionally active fragments of disordered proteins, implication for neurodegenerative diseases, Protein Expression and Purification, 35:366-372, 2004).

Phosphorylated and unphosphorylated tau proteins 2N4R and tau151-391 and sarcosyl insoluble PHF-tau were analyzed by immunoblotting using DC2E2. Soluble tau proteins were diluted with an equal volume of 2×SDS (sodium dodecylsulfate) sample loading buffer (with β-mercaptoethanol) (Laemmli, 1970) and 250 ng of proteins were loaded per lane. For insoluble PHF-tau, the pellets were dissolved in 1×SDS-sample loading buffer, in 1/50 volume of the soluble fraction used for the preparation of the insoluble tau fraction. Then, equal volumes of soluble tau and sarkosyl-insoluble tau fractions were used for immunoblotting, which corresponded to 15 μg of total protein in the soluble fraction (see Filipcik et al. 2010). Samples were heated at 95° C. for 5 min, loaded onto 5-20% gradient SDS polyacrylamide gels, and electrophoresed in a Tris-glycine-SDS buffer system for 40 minutes at 25 mA. Proteins were transferred to a polyvinylidene fluoride (PVDF) membrane (1 h at 150 mA in 10 mM CAPS, pH 12). After the transfer, the membranes were blocked in 5% non-fat dry milk in phosphate-buffered-saline (PBS; 136.89 mM NaCl, 2.7 mM KCl, 8.09 mM Na2HPO4, 1.47 mM KH2PO4) for 1 h at RT, and then incubated for 12 h with DC2E2 hybridoma culture supernatant, followed by three washes with large volumes of PBS-T (1% Tween 20). The membranes were incubated (1 h at room temperature) with HRP conjugated goat anti-mouse Ig (DAKO, Denmark), diluted 1:3,000 with 1% non-fat dry milk in PBS, as a secondary antibody. This incubation was followed by washing (three times) with 0.1% Tween 20 in PBS. The blots were developed with SuperSignal West Pico Chemiluminescent Substrate (Pierce, U.S.A), and the protein signals detected using a LAS3000 imaging system (FUJI Photo Film Co., Japan). The chemiluminescence signal intensities were quantified using AIDA (Advanced Image Data Analyzer, Raytest, Straubenhardt, Germany) software.

DC2E2 recognized both forms of tau proteins, in vivo phosphorylated tau 2N4R, 151-391 and wild type (unphoshorylated) forms of tau 2N4R and tau151-391 (FIG. 3).

Furthermore, it is noteworthy that DC2E2 recognized the A68 triplet, a characteristic feature of pathological tau (PHF-tau) in AD neurofibrillary degeneration. These results demonstrate that DC2E2 can recognize and bind different type of tau proteins, pathological tau isolated from AD brain, physiological tau (2N4R), truncated tau151-391 and its phosphorylated forms. Thus, DC2E2 recognized epitopes independent of epitope phosphorylation. Disclosed binding properties of DC2E2 suggest the possibility of using the antibody as diagnostic tool for AD.

### Example 5

### Sequencing of DC2E2 Variable Regions

Determination of the nucleotide and amino acid sequences of the light and heavy chain variable regions of DC2E2 (FIG. 5). The nucleotide sequence of DC2E2 variable regions (FIGS. 5A and 5C) was determined by DNA sequencing of cDNA synthesized using total RNA extracted from the mouse hybridoma cell line DC2E2 (ATCC), which expresses the DC2E2 monoclonal antibody. Total RNA was extracted using RNeasy Mini Kit (Qiagen, Germany). Synthesis of the first strand cDNA was carried out using the “High capacity cDNA reverse transcription” kit according to the manufacturer's protocol (Applied Biosystems, USA). The composition of the reagents for the 2× reverse transcription master-mix was as follows (quantities per 20 μL reaction): 2 μL of 10×RT buffer; 0.8 μL of 25×dNTP Mix (100 mM); 2 μl of 10×RT Random Primers (50 μM); 1 μL of MultiScribe™ Reverse Transcriptase (50 U/μL); 4.2 μL of nuclease-free H₂O. For reverse transcription, 10 μL of the 2× reverse transcription master-mix was mixed with RNA sample (1 μg/10 μL) and cDNA was synthesized under the following conditions: 10 min at 25° C., 120 min at 37° C., 5 min at 85° C., and final cooling to 4° C. The genes encoding the variable regions of the light and heavy chains were apmplified by polymerase chain reaction (PCR) using Phusion® High-Fidelity DNA Polymerase (Thermo Fisher Scientific, USA). The forward primers (M13-L6 5′-TGTAAAACGACGGCCAGTATGAGGTKCYYTGYTSAGYTYCTGRGG-3′ and M13-H1 5′-TGTAAAACGACGGCCAGTATGAAATGCAGCTGGGTCATSTTCTTC-3′) for the light and heavy chain, respectively, were selected after screening a library of mouse immunoglobulin signal sequence forward primers. Using signal sequence forward primers has the advantage of amplifying the whole V-gene of both light and heavy antibody chains, without bias introduced when the beginning of the variable region is being used for primer design. The reverse primers for the light and heavy chains (M13-KC 5′-CAGGAAACAGCTATGACCACTGGATGGTGGGAAGATGG-3′ and M13-CG1 5′-CAGGAAACAGCTATGACCCAGTGGATAGACAGATGGGGG-3′) were derived from kappa and IgG1 chains constant regions, respectively.

The PCR products were sequenced and the resulting DNA sequences of the variable regions of the light and heavy chains of DC2E2 are shown in FIGS. 5A and 5C, respectively. Complementarity determining regions (CDRs) are underlined in the DC2E2 light and heavy chains protein sequences (FIGS. 5B and 5D, respectively). CDRs and framework regions (FR) were identified according to the ImMunoGeneTics (IMGT) numbering system (see, e.g., Lefranc M. P. The IMGT unique numbering for immunoglobulins, T-cell receptors, and Ig-like domains. The Immunologist, 7, 132-1 36, 1999 (1999)).

### Example 6

### Preparation of Hybridoma Cell Lines Producing Monoclonal Antibodies Against Insoluble Tau Species In Human Alzheimer's Disease, Screening Of Monoclonal Antibodies By ELISA, And Initial Characterization Of Monoclonal Antibody DC2E7

Sarcosyl-insoluble tau (PHF-tau) from AD brain was used as an immunogen for immunization of Balb/c mice. Sarcosyl insoluble tau complexes were isolated from human AD brain (frontal cortex, Braak stage VI, Netherlands brain bank) using the sarkosyl method (Greenberg and Davies, A preparation of Alzheimer paired helical filaments that diplays distinct tau proteins by polyacrylamide gel electrophoresis, PNAS, 87:5827-31, 1990) as described in Example 4.

Six-week-old Balb/c mice were primed subcutaneously with approximately 20-30 μg of insoluble tau protein isolated from human Alzheimer's brain tissue in complete Freund's adjuvant (SIGMA) and boosted five times at four-week intervals with 20-30 μg of the same antigen in incomplete Freund's adjuvant. Three days before the fusion, mice were injected intravenously with 20-30 μg of the same antigen in PBS. Spleen cells from immunized mice were fused with NS/0 myeloma cells according to the method of Kontsekova et al., The effect of postfusion cell density on establishment of hybridomas, Folia Biol. 34, 18-22 (1988). Splenocytes were mixed with NS/0 myeloma cells (ratio 5:1) and fused for 1 minute in 1 ml of 50% polyethylene glycol (PEG) 1550 (Serve) in serum free Dulbecco's modified Eagle's medium (DMEM) supplemented with 10% dimethyl sulphoxide. The fused cells were resuspended in DMEM containing 20% horse serum, L-glutamine (2 mM), hypoxanthine (0.1 mM), aminopterin (0.04 mM), thymidine (0.016 mM), and gentamycin (40 U/ml), at a density of 2.5×10⁵ spleen cells per well on 96-well plates.

The cells were incubated for 10-14 days at 37° C. and growing hybridomas were screened for the production of anti PHF-tau-specific monoclonal antibodies by an enzyme-linked immunosorbent assay (ELISA). Microtiter plates were coated overnight with PHF-tau (5 μg/ml, 50 μl/well) at 37° C. in PBS. The plates were blocked with 1% nonfat dried milk to reduce nonspecific binding, washed with PBS-0.05% Tween 20, and incubated with 50 μl/well of hybridoma culture supernatant for 1 hr at 37° C. Bound monoclonal antibodies were detected with sheep anti-mouse immunoglobulin (Ig) conjugated with horse radish peroxidase (HRP, DAKO). The reaction was developed with TMB one (Kem-En-Tec Diagnostics) as a peroxidase substrate and stopped with 50 μl of 2 M H₂SO₄. Absorbance at 450 nm was measured using a Powerwave HT (Bio-Tek). Readouts with an absorbance value of at least twice the value of the negative controls (PBS) were considered positive. Positive hybridoma cultures were further subcloned in soft agar according to the procedure described in Kontsekova et al., One-step method for establishing 8-azaguanine-resistant hybridomas suitable for preparation of triomas, J. Immunol. Methods, 145, 247-250, (1991).

The monoclonal antibody DC2E7 (produced by the mouse hybridoma cell line deposited with the American Type Culture Collection, with the ATCC Patent Deposit Designation PTA-124992) was identified among the positive hybridoma cultures. DC2E7 was further characterized as described below. The antibody isotype was determined to be murine IgG2a by ELISA (FIG. 4) using a mouse Ig isotyping kit (ISO-2, SIGMA).

### Example 7

### Sequencing of DC2E7 Variable Regions

Determination of the nucleotide and amino acid sequences of the light and heavy chain variable regions of DC2E7 (FIG. 6). The nucleotide sequence of DC2E7 variable regions (FIGS. 6A and 6C) was determined by DNA sequencing of cDNA synthesized using total RNA extracted from the mouse hybridoma cell line DC2E7 (ATCC), which expresses the DC2E7 monoclonal antibody. Total RNA was extracted using RNeasy Mini Kit (Qiagen, Germany). Synthesis of the first strand cDNA was carried out using the “High capacity cDNA reverse transcription” kit according to the manufacturer's protocol (Applied Biosystems, USA). The composition of the reagents for the 2× reverse transcription master-mix was as follows (quantities per 20 μL reaction): 2 μL of 10×RT buffer; 0.8 μL of 25×dNTP Mix (100 mM); 2 μl of 10×RT Random Primers (50 μM); 1 μL of MultiScribe™ Reverse Transcriptase (50 U/μL); 4.2 μL of nuclease-free H2O. For reverse transcription, 10 μL of the 2× reverse transcription master-mix was mixed with RNA sample (1 μg/10 μL) and cDNA was synthesized under the following conditions: 10 min at 25° C., 120 min at 37° C., 5 min at 85° C., and final cooling to 4° C. Amplification of the genes encoding the variable regions of the light and heavy chains was done by polymerase chain reaction (PCR) using Phusion® High-Fidelity DNA Polymerase (Thermo Fisher Scientific, USA). The forward primers (M13-L12 5′-TGTAAAACGACGGCCAGTATGAAGTTTCCTTCTCAACTTCTGCTC-3′ and M13-H5 5′-TGTAAAACGACGGCCAGTATGGACTCCAGGCTCAAMAGTTTTCCTT-3′ were selected after screening a library of mouse immunoglobulin signal sequence forward primers. The usage of signal sequence forward primers has the advantage to amplify the whole V-gene of both light and heavy antibody chains, without biases introduced when the beginning of variable region is being used for primer design. The reverse primers for the light and heavy chains (M13-KC 5′-CAGGAAACAGCTATGACCACTGGATGGTGGGAAGATGG-3′ and M13-CG2a 5′-CAGGAAACAGCTATGACCCAGTGGATAGACCGATGGGGC-3′ were derived from kappa and IgG2a chains constant regions, respectively.

The PCR products were sequenced and the resulting DNA sequences of variable regions of light and heavy chains of DC2E7 are shown in FIGS. 6A and 6C, respectively. Complementarity determining regions (CDRs) are underlined in the DC2E7 light and heavy chains protein sequences (FIGS. 6B and 6D, respectively). CDRs and framework regions (FR) were identified according to the ImMunoGeneTics (IMGT) numbering system (see, e.g., Lefranc M. P. The IMGT unique numbering for immunoglobulins, T-cell receptors, and Ig-like domains. The Immunologist 7, 132-1 36, 1999 (1999)).

### Example 8

### Monoclonal Antibody DC2E7 Is Specific For Phosphorylated Forms Of Tau Protein

Recombinant full length tau 2N4R, phosphorylated 2N4R, PHF-tau and fetal tau were used for further characterization of binding activity of monoclonal antibody DC2E7 by immunoblotting. Recombinant human tau isoform 2N4R was prepared as described in Example 1. PHF-tau was prepared as described in Example 4.

Fetal rat tau extraction and purification was done essentially as described in Ivanovova et al., High-yield purification of fetal tau preserving its structure and phosphorylation, J. Immunol. Methods, 339:17-22, 2008, using 1% perchloric acid. Brain tissue obtained from 1-7 day old rat pups was homogenized in ice-cold 1% perchloric acid (1.5 g tissue per 5 ml of perchloric acid, Applichem) and allowed to stand on ice for 20 min. The homogenate was spun at 15,000×g for 20 min, and the clear supernatant was concentrated and simultaneously the buffer was changed to washing buffer (20 mM Tris, pH 7.4, 150 mM NaCl, 0.1% Tween 20) using an Amicon Ultra Centrifugal Filter device (Millipore). The filtered extract were loaded at a flow rate of 0.2 ml/min onto a Poly-Prep column 010/10 (GE Healthcare) packed with Sepharose carrying immobilized pan-tau mAb DC25. Unbound proteins were washed off with 10-15 ml washing buffer until the absorbance of the eluting fractions (at 280 nm) became stable. Fetal tau, bound to mAb DC25, was eluted with 0.1 M glycine, pH 2.6. Eluted 0.5 ml fractions were immediately neutralized with 50 μl of 1M Tris-HCl, pH 9, and assayed by SDS-PAGE. Fractions containing fetal tau were concentrated using Amicon Ultra Centrifugal Filter devices (Millipore) with simultaneous buffer exchange to PBS. Fetal tau purified by affinity chromatography was precipitated according to Chen et al., (2005) by addition of four volumes of ice cold acetone containing 10% trichloroacetic acid. The mixture was incubated at −20° C. for 2 hours and centrifuged at 15,000×g for 20 min at 2° C. The supernatant was discarded and the precipitate was resuspended in 1 ml of ice-cold acetone, allowed to stand on ice for 20 min and again centrifuged as above. The resulting pellet was dried at room temperature and dissolved in a volume of PBS equal to that volume before precipitation.

Full length tau isoform 2N4R, sarcosyl insoluble PHF-tau and fetal tau were analyzed by immunoblotting using DC2E7 as described in Example 4. In immunoblotting DC2E7 antibody recognized PHF-tau and fetal tau (FIG. 7). Both types of tested tau were phosphorylated, but at different levels of phosphorylation. No immunoreactivity was observed on recombinant human tau 2N4R. On the other hand, the antibody bound in vitro phosphorylated versions of human tau isoform 2N4R. The results indicate that DC2E7 recognizes an epitope which is phosphorylated, thus the antibody is specific for phosphorylated tau species. The epitope recognized by the antibody is present in phosphorylated PHF-tau, fetal tau and in vitro phosphorylated tau. Accordingly, DC2E7 is capable of distinguishing phosphorylated tau derived from Alzheimer's disease brain tissue (PHF-tau) from physiological tau (2N4R).

### Example 9

### Mapping Of The DC2E7 Phosphoepitope Using Recombinant Tau Deletion Mutants, Tau Point Mutants, and Tau-Derived Peptides

The immunoreactivity of DC2E7 suggested that antibody recognizes epitope on phosphorylated tau (fetal tau, PHF-tau, in vitro phosphorylated tau, see supra). Therefore, to determine the epitope, phosphorylated deletion mutants of tau protein were used (FIG. 8). Phosphorylation of tau deletion mutants was done using kinase extract as described in Example 4.

Phosphorylated deletion mutants of human tau protein 2N4R (tau 1-296, tau 188-441, tau 221-441, tau151-391, tau 122-227) were used for the localization of the epitope of DC2E7 in immunoblotting (see Example 4). Immunoblotting analysis demonstrated that DC2E7 recognized all of the following deletion mutants: taul-296, tau188-441, tau151-391, tau122-227, except tau protein 221-441 (FIG. 9). These results indicate that the DC2E7 phospho-epitope lies in proline reach region, between amino acid residues 188-227.

The abovementioned mapping of the DC2E7 epitope with tau deletion mutants suggested an epitope between Pro188-Ala227. In this region of tau there are 11 phosphorylation sites (Seri 91, Tyr197, Ser198, Ser199, Ser202, Thr205, Ser208, Ser210, Thr212, Ser214, Thr217) detected on PHF-tau (Hanger et al., 2009, FIG. 10). Four of them (Ser198, Ser199, Ser202, Thr217) were found also on fetal tau (Morishima-Kawashima et al., 1995, FIG. 10). Because DC2E7 recognizes also fetal tau, the first focus was on phospho-sites that are present and confirmed in fetal tau (Ser198; Ser199; Ser202; Thr217).

To define the exact phosphosite(s) for DC2E7, mutated forms of tau 2N4R with single point mutations in which Serine and Threonine residues were replaced by Alanine were generated. Insertion of the desired point mutations (Ser198Ala; Ser199Ala; Ser202Ala; Thr217A1a) into human 2N4R tau protein was done using QuickChange site directed mutagenesis kit (Agilent Technologies, USA, CA) according the manufacturer's instructions. 5-50 μg of plasmid containing coding sequence for wild-type 2N4R tau protein and 125 ng of each primer were used in one 50 μl reaction. Cycling parameters were as follows: 95° C. for 30 seconds, then PCR continued with cycle which was repeated 16 times: denaturation 95° C. for 30 seconds, annealing 55° C. for 60 seconds, elongation 68° C. for 6 min. The whole reaction was digested with Dpnl, which is specific for methylated DNA, to eliminate parental plasmid. 50 μl of supercompetent Escherichia coli XL-1 Blue (provided with the mutagenesis kit) were transformed with 1 μl of the reaction and spread onto LB-agar plates supplemented with ampicillin. Plasmids were isolated from grown colonies and verification of the mutagenesis was done using DNA sequencing. Plasmids with desired mutations were used for production of recombinant proteins as described in Example 1.

Prepared tau mutants were purified as described in Example 1, phosphorylated by kinase extract from the rat brain and stained with DC2E7 in immunoblotting. Phosphorylation of deletion mutants of tau induced shift in molecular weight, thus all proteins were phosphorylated, as shown staining with pan tau antibody DC25 (epitope 347-353 aa, AXON Neuroscience, SE) (FIG. 11). Analysis of phosphorylated tau point mutant demonstrated that DC2E7 detected all single mutations (Ser198Ala, Ser199Ala, Ser202A1a) but Thr217Ala (FIG. 11). These results suggested, that phospho-threonine at position 217 creates key part of the epitope recognized by antibody DC2E7.

However, other phospho-sites which are located in proximity the phospho-site Thr217 could be a part of the epitope, or may be involved into creation of the epitope. Therefore, the possible impact of phospho-sites Thr205, Ser208, Ser210, Thr212, Ser214, Ser231 on epitope recognized by DC2E7 was examined. Prepared mutant tau proteins with single point mutations (Thr205Ala, Ser208Ala, Ser210Ala, Thr212Ala, Ser214Ala, Ser231A) were phosphorylated by kinase extract and tested in immunoblotting with DC2E7 (FIG. 11). The immunoblotting analysis demonstrated that the antibody recognized all tau proteins carrying the point mutations (FIG. 11). Thus, phospho-sites located in the proximity of Thr217 did not affect the epitope recognized by DC2E7.

These mapping experiments suggested the presence of phospho-threonine 217 within the epitope of DC2E7. In order to examine the effect of residues in the region of DC2E7 epitope on antibody binding, synthetic peptides of different lengths carrying different phospho-sites were analyzed in competitive ELISA. Peptides (tau 210-224/pT212/pT217, 210-222/pT212/pT217, 210-221/pT212/pT217, 210-220/pT212/pT217, 210-219/pT212/pT217, 210-218/pT212/pT217, tau201-230/pT212, tau 193-222/p5208, 193-222/p5214, tau 193-231/pS208/pS210/pT212/pS214/pT217, tau 193-231/pS202/pS205/pT212/pT220, tau 201-229), were synthesized by EZBiolabs (USA) with purity higher than 95%. Each synthetized peptide contained phospho-site(s) located around the residue Thr217, except of unphosphorylated peptide tau 201-229 (used as negative control). As positive control, in vitro phosphorylated tau151-391 was used.

Next, all peptides were analyzed for their ability to compete with in vitro phosphorylated tau151-391 for binding to DC2E7 by competition ELISA. ELISA plates (Nunc Medisorp, Thermo Scientific, Denmark) were coated overnight at 37° C. with 50 μl/well of PHF-tau, diluted 200× in PBS. The coated plates were washed 5 times with PBS/Tween 20 (0.05% v/v), and blocked with PBS/Tween 20 for 1 h at 25° C. Each of the peptides was separately dissolved in PBS at a final concentration of 1 mM. Serial dilutions (2.5×) of peptides in PBS/Tween 20 were prepared in polypropylene microtiter plates with conical well bottom (Greiner) within the concentration range of 200 μM; 80 μM; 32 μM; 12.8 μM; 5.12 μM; 2.048 μM; 0.8192 μM; 0.32768 μM). The monoclonal antibody DC2E7 was diluted to a concentration of 0.6 μg/ml in PBS and 60 μl of this diluted antibody was added into each well to serial dilution of peptides resulting in 120 μl/well of mixture. The antibody/peptide mixtures were incubated for 1 hr at 25° C. on a rotating platform set to 230 rpm. 50 μl/well of antibody/peptide mixtures were transferred from polypropylene plates into PHF-tau coated and PBS/Tween 20 blocked ELISA plates (in duplicates) and incubated for 1 hr at 25° C. The plates were washed 5× times with PBS/Tween 20 and incubated with 50 μl/well of Polyclonal goat anti-mouse immunoglobulins/HRP (Dako) diluted 1:1000 in PBS/Tween 20 for 1 hr at 25° C. After washing, the plates were then incubated with 50 μl/well of 1 mg/2 mL o-PDA (o-phenylenediamine, Sigma) in 0.1 M Na-Acetate pH=6.0 (Roth) supplemented with 1.5 μl/2 ml of 30% H₂O₂ (Sigma) for 20 minutes at 25° C. in dark. The reaction was stopped by adding 50 μl/well of 2M H₂SO₄ (Merck) followed by reading the plates at 492 nm (Powerwave HT, Bio-Tek).

All analyzed peptides that encompassed phosphorylated threonine 217 competed with phosphorylated tau151-391 for binding to DC2E7 (FIG. 12). In summary, the DC2E7 binds a tau epitope that includes a phosphorylated threonine 217. No other phospho-sites appeared to impact antibody immunoreactivity. But, notably, shortening of the C terminal part of the tested peptides from 224 to 220 amino acids (210-220/pT212/pT217, 210-219/pT212/pT217, 210-218/pT212/pT217) led to the loss of competing activity despite the presence of phosphorylated threonine 217. The data thus suggest that DC2E7 binds a phosphoepitope on human phosphorylated tau of 12 amino acids which comprise the residues 210-SRTPSLPTPPTR-221, where at least the threonine 217 is phosphorylated.

### Example 10

### Monoclonal Antibodies DC2E2 and DC2E7 Recognize Pathology In Human Alzheimer's Disease Brain and Tauopathies

This example shows that DC2E7 and DC2E2 recognise neurofibrillary lesions in Alzhemer's disease. The following brain areas were used for immunohistochemical study: hippocampus and entorhinal cortex from Alzheimer's disease (Braak stage 6), FTD (Pick's disease), and control brains (Braak stage 1 and 3), caudate nucleus from corticobasal degeneration (CBD) and putamen/caudate nucleus from progressive supranuclear palsy (PSP). The brain tissue paraffin blocks were obtained from Amsterdam brain bank.

The brains blocks embedded in paraffin were cut on a microtome (Leica RM2255) to get 8 μm thick sections. The sections were placed on HistoBond slides (Marienfeld, Germany). For immunohistochemistry sections were pre-treated with formic acid (98% for 1 min at 4° C. or 80% for 1 hour) and heat (autoclave, 121° C., 20 min.), followed by overnight incubation with primary antibodies (AT8 1:1000, DC2E7 1:10 000, DC2E2 1:200). All sections were incubated with anti-mouse biotinylated secondary antibody at room temperature for 1 hour and with avidin-biotin peroxidase-complex for 1 hour. The immunoreaction was visualised with VIP (Vectastain Elite ABC Kit, Vector Laboratories, CA, USA) and counterstained with methyl green (Vector Laboratories).

Immunohistochemical analyses revealed that both DC2E7 and DC2E2 recognized tau pathology in Alzheimer's disease and other tauopathies (FIG. 13) in a similar way as monoclonal antibody AT8 which is considered to be golden standard for histopathological staining. DC2E7 and DC2E2 stained neurofibrillary pathology in the hippocampus of AD patient, Pick's bodies in the dentate gyrus of FTD patient, glial tau pathology in caudate nucleus of patients suffering either from CBD and PSP. DC2E7 and DC2E2 do not recognize tau pathology in normal brain fulfilling criteria for Braak stage 1, in prodromal stage (Braak stage 3) both antibodies identified in the hippocampus neurofibrillary pathology, in the full blown AD antibodies visualized extensive tau pathology in the form of neurofibrillary tangles, neuropil threads and neuritic plaques (FIG. 14). By using higher magnification, it was demonstrated that the antibodies bound neurofibrillary tangles in AD, Pick's bodies in FTD and glial tau pathology in PSP and CBD (FIG. 15).

### Example 11

### DC2E7 ELISA

### Preparation of Standard for DC2E7 ELISA

In vitro phosphorylated tau151-391 was purified by affinity chromatography. Two affinity columns were prepared: DC2E7 and DC190 (epitope 368-376, AXON Neuroscience, SE). Antibodies were purified according method described in Example 3. Antibodies were coupled to CnBr-activated Sepharose 4B (GE Healthcare, #17-0430-01) according to the manufacturer recommendation. In vitro phosphorylation reaction (see Example 4) was dialyzed against PBS at 4° C. (3×100-fold excess). All purification steps were performed at 4° C. DC2E7 column was washed with 3×5 ml of WBNP0.1 buffer (50 mM Tris pH 7.4, 150 mM NaCl and 0.1% NP40). In vitro phosphorylation reaction was diluted 4-fold with ice cold WBNP1 buffer (50 mM Tris pH 7.4, 150 mM NaCl and 1% NP40) and filtered through 0.2 μm filter. Sample was applied onto DC2E7 column. After sample entered resin column was washed as follows: 2×5 mL with WBNP1 buffer, 2×5 mL with WBNP0.1 buffer, 1×5 mL with 50 mM Tris.HCl pH 7.4, 150 mM NaCl. Bound proteins were eluted with 3×2 ml of elution buffer (100 mM glycin/HCl, pH 2.8). Eluted proteins were immediately adjusted to pH 7-8 with 1 M Tris.HCl, pH 9, and diluted 1:1 with WBNP0.1 buffer. Sample was applied on DC190 affinity column and purified as described for DC2E7 affinity purification. Eluted proteins were dialyzed against PBS and concentration was spectrophotometrically estimated. Purified protein was designated “DC2E7 calibrator”.

DC2E7 ELISA was set up in high sensitive format, digital ELISA, using Simoa-HD1 analyzer (Quanterix). Reagents for digital ELISA were prepared according to the Quanterix Homebrew Assay Development Guide with following details. DC2E7 antibody was used as a capture antibody and DC2E2 antibody was used as a detector antibody. DC2E7 was coupled to magnetic beads (Quanterix) at concentration of 0.5 mg/mL. Detector antibody was prepared by biotinylation of DC2E2, whereby 120-fold excess of Biotin, EZ-Link™ NHS-PEG₄-Biotin (Thermo Scientific, #21329) over antibody concentration was used. DC2E7 calibrator was diluted in calibrator diluent (20 mM sodium phosphate pH 7.4, 137 mM NaCl, 2.7 mM KCl, 2% BSA and 0.01% casein) in serial two-fold dilutions starting from 100 μg/mL, following by 50, 25, 12.5, 6.25, 3.13, 1.56 and 0 μg/ml. Prepared calibrator concentrations were mixed in a 3:1 ratio with sample diluent (80 mM sodium phosphate pH 7.4, 548 mM NaCl, 10.8 mM KCl, 0.04% casein and 0.4% Tween 20). CSF samples from control individuals were also diluted with sample diluent, as previously described. Spike recovery of CSF sample was performed using spikes of CSF with 10, 5, 2 and 0 μg/ml of DC2E7 calibrator. Diluted calibrators and samples were pipetted into 96 well plate, inserted in Simoa HD1 analyzer and also capture antibody DC2E7 beads diluted in bead diluent, detector DC2E2 diluted in detector diluent to 1.2 μg/ml, SBG diluted in SBG diluent to 200 μM and substrate RGP were inserted in analyzer (all buffers, SBG and RGP were obtained from Quanterix). Assay was programmed in Simoa 1.5 software and analysis was performed. After analysis evaluation was done by Graphpad Prism and/or by software included in Simoa 1.5 software. An example of a calibration curve is shown on FIG. 16. An example of a spike recovery experiment is shown on FIG. 17.

### Example 12

### DC2E7 Digital ELISA Significantly Distinguishes Between Alzheimer's Disease Patients And Control Individuals

DC2E7 digital ELISA was used for analysis of CSF samples from Alzheimer's disease patients (n=20) and healthy individuals (n=20) (FIG. 18). Analysis demonstrated that DC2E7 digital ELISA distinguished Alzheimer's disease patients from control individuals with very high significance. Area of ROC curve was very close to 1. At a concentration >4.43 μg/mL the assay gives 95% sensitivity and 89.5% specificity and at concentration >6.19 μg/mL, sensitivity is 95% and specificity 100%.

### Example 13

### DC2E7 Digital Elisa Significantly Distinguishes Between Alzheimer's Disease Patients And Patients Suffering From Other Tauopathies

DC2E7 digital ELISA was used for analysis of CSF samples from Alzheimer's disease patients (n=6) and frontotemporal dementia (n=16) (FIG. 19). Analysis demonstrated that DC2E7 digital ELISA distinguished Alzheimer's disease patients from patients having other tauopathies with very high significance. Area of ROC curve is 0.97. At concentration >3.89 μg/ml the assay gives 93.8% sensitivity and 100% specificity.

### Example 14

### DC2E7 Recognizes Insoluble Tau Species In Alzheimer's Disease And Human Tauopathies

Previous results showed that DC2E7 recognized an epitope which occurs in phosphorylated tau. Therefore, the immunoreactivity of DC2E7 to phosphorylated tau protein which occurs in other tauopathies was analyzed.

Insoluble tau complexes (abnormal tau forms) were isolated from human AD brain (Braak stage V, frontal cortex, Amsterdam Brain bank), corticobasal degeneration (London brain bank, frontal cortex) and FTD (Amsterdam Brain bank) as described in Example 4. Extracted insoluble tau proteins were analyzed in immunoblotting using DC2E7 as described in Example 4.

Immunoblot analyses of brains from patients with AD and aforementioned tauopathies revealed that insoluble tau fractions were detectable using DC2E7. The results suggest that the pathological tau aggregates in tauopathies are composed of hyperphosphorylated tau similar to PHF-tau in AD (FIG. 20). Moreover, DC2E7 recognized three predominant PHF-tau-like bands of 60, 64, and 68 kDa (A68 triplet) in CBD and FTD similar to that of AD.

### Example 15

### pT217 Tau Digital ELISA Assay Distinguished AD Patients From FTD and Healthy Subjects With High Sensitivity And Specificity

A pT217 tau digital assay using antibody DC2E7 was used to analyze CSF from AD patients (n=30), FTD patients (nfPPA, n=14; svPPA, n=10; bvFTD, n=10, PSP, n=19; and CBD, n=15), and healthy individuals (n=30). The assay was compared to an INNOTEST Phospho-Tau(181P) assay.

Analysis demonstrated that the pT217 tau digital ELISA assay distinguished AD patients from FTD and healthy subjects. The assay better separated AD from FTD/controls (>5.37 μg/ml, 94.1% sensitivity, 91.7% specificity, FIG. 21), as compared to the Innotest Phospho-Tau(181P) assay (>52 μg/ml, 78.0% sensitivity, 100% specificity. (FIG. 21). The assay was also slightly better at distinguishing AD patients from healthy subjects (>3.855 μg/ml, AUC 0.96, 95% CI, 0.89-0.99, 95% sensitivity, 89.7% specificity) than the p-tau 181 assay (>52 μg/ml, AUC 0.93, 95% CI, 0.85-0.97, 92% sensitivity, 87.5% specificity) (FIG. 22). Comparing the high correlation between pT217 and pT181 assays in the AD group (P<0.0001, Spearman coefficient r=0.8226) to the low correlations observed in healthy controls (P<0.5, Spearman coefficient r=0.4032) and FTD (P=0.387, Spearman coefficient r=0.232) suggests that the pT217 tau species in CSF may provide an AD specific biomarker in CSF. No benefit was observed for using of ratio INNOTEST Aβ42/pT217 tau when compared to pT217 tau itself.

High correlation was observed between pT217 tau and pT181 tau assays in the AD group (P<0.0001, Spearman coefficient r=0.8226) and the low correlations was observed in non-demented controls (P<0.5, Spearman coefficient r=0.4032).

No correlation was observed between pT217 tau and pT181 tau assays in patients from FTD (P=0.7517, Spearman coefficient r=0.08929); whereby strong correlation was observed between pT181 and HTAU assays in the same FTD patients (P=0.0019, Spearman coefficient r=0.7321)

All these results suggest that the pT217 tau species in CSF may provide an AD specific biomarker in CSF.

### Example 16

### pT217 Tau Digital ELISA Assay Distinguished AD Patients From FTD And Healthy Subjects With High Sensitivity And Specificty Using Different Calibrators

The pT217 tau digital ELISA assay can distinguish between subjects with mild cognitive impairment (MCI) and healthy subjects. The pT217 tau concentrations in cerebrospinal fluid (CSF) are similar for subjects with MCI and AD (FIG. 23A). Three subjects with MCI later developed AD.

The pT217 tau assay was used to test CSF samples from subjects with other neurological disorders including multiple sclerosis (MS), Parkinson's disease (PD), amotrophic lateral sclerosis (ALS), and frontotemporal dementia (FTD), where tau pathology may be present (FIG. 23B). The FTD patients represent quite heterogeneous populations, some of those patients suffered from progressive supranuclear palsy (PSP) or corticobasal degeneration (CBD), while the others developed symptoms typical for primary progressive aphasia (PPA). The pT217 tau digital ELISA assay distinguishes between AD and the other neurological disorders (cut-off 9.3 μg/ml, specificity, 94%, sensitivity 100%).

The pT217 tau digital ELISA assay can distinguish AD patients from FTD patients and healthy subjects with similar sensitivity and specificity regardless of the chosen calibrator. Distribution of pT217 tau in CSF samples from AD patients, FTD patients and healthy subjects using either in vitro phosphorylated tau protein or synthetic peptide showed similar sensitivity and specificity. Using the phosphorylated tau protein calibrator, the assay exhibited 94.1% sensitivity and 91.7% specificity (>5.37 μg/ml). Using the 2E7 peptide (2E7pep), the assay exhibited 93.9% sensitivity and 90.0% specificity (>305 μg/ml) (FIG. 29). 2E7pep possessed the following amino acid sequence: GQKGQANATRIPAKGGGSGGGSGGGSSRTPSLPpTPPTREPK. The first underlined sequence represents an epitope of the DC2E2 antibody, the second underlined sequence represents an epitope of the DC2E7 antibody (first 3 amino acids and last amino acids are derived from tau protein, while the sequence (GGGS)₃ is a linker connecting the epitopes of DC2E2 and DC2E7).

### Example 17

### Optimization of Antibody DC2E7 By Recombinant Technologies

The affinity of the DC2E7 antibody was optimized by ribosome display and phage display technologies. First, cDNA encoding an scFv format of antibody DC2E7 was generated by amplification and cloning from a DC2E7 hybridoma cell line. The gene for the scFv DC2E7 was then mutated by error-prone PCR and a library of mutated scFv DC2E7 fragments was affinity selected against 2E7pep by ribosome display (Hanes et al., 1998) and phage display technologies (Harrison at al., 1996)

After four rounds of selection, isolated scFv's fragments were cloned into an expression vector such that scFvs were fused to a myc-tag and expressed in E. coli JM109 (Sanmark et. al., 2015). Bacterial lysates of 30 colonies containing mutated DC2E7 scFv were tested using a peptide competition immunoassay. The immunoassays were performed in 96 well microtiter plates coated with 100 μl/well of 0.01 μg/ml of 2E7pep in PBS at 4° C. overnight. Plates were washed 4× and blocked in PBS-T for 30 min at room temperature. Binding was performed with 100 μl of 10× diluted bacterial lysate in PBS-T, containing anti-myc antibody diluted 10,000×, on a shaking platform for 1 hour at RT. The wells were washed four times with PBS-T and competition with a 2E7pep at a concentration of 10 μg/ml per lysate was performed for 2 hours at RT on a shaking platform. Then, plates were washed 4× with PBS-T and incubated with streptavidin-HRP conjugate diluted 1:10000 in PBS-T for 1 hour at RT. The plates were washed 4×PBS-T and developed using a TMB substrate for 5 min before the colorimetric reaction was stopped using 1 M H₂SO₄. The absorbance at 450 nm was read using a microtiter plate absorbance reader.

Absorbance for the scFvs generated by ribosome display and phage display are shown in FIG. 24. scFv K+ represents unmutated DC217. All scFv showing signal above the line corresponding to scFv K+ are expected to have higher affinity (labeled with an asterisk), as compared to the original DC217 scFv.

Alignments of the heavy and light chain amino acid sequences of the scFvs isolated by ribosome display and phage display (“PD” at the end of a clone name indicates it was obtained by phage display) are shown in FIG. 25. VL (FIG. 25A) and VH (FIG. 25B) sequences are shown. Antibodies with improved binding relative to DC2E7 are shown in FIG. 25C (light chain) and FIG. 25D (heavy chain). The first line represents the original DC2E7 antibody. Residues identical to the sequence in DC2E7 are represented by dots. Numbering of amino acid residues in VL and VH and the labeling of CDRs is according to the IMGT numbering system.

### Example 18

### Comparison of Antibodies DC2E7, DC149, and DC807

Antibody DC149 was generated in a similar manner to what was described in Example 2, with the following difference: Mice were immunized with a double phosphorylated peptide at positions Thr212 and Thr217, i.e., CSRpTPSLPpTPPTREPK (210-224 of 2N4R tau) conjugated via the first cysteine to KLH. Screening was performed by ELISA as described in Example 2, whereby DC149 was identified by specific binding to peptide comprising SRTPSLPpTPPTREPK (i.e., the same peptide mono-phosphorylated at Thr217 that DC2E7 bound) and DC807 by specific binding to the double-phosphorylated peptide. The DC149 and DC807 antibodies did not bind to an unphosphorylated peptide or to unphosphorylated tau protein, while DC807 also did not bind to mono-phosphorylated peptides.

DC2E7, and DC149 an DC807 were analyzed in a classical ELISA assay using a similar setup to what was described in example 10, whereby 2E7pep calibrator was used. All three antibodies bound to tau peptide phosphorylated at least on Thr217 with very similar affinities (FIG. 26).

Amino acid sequences for antibodies DC217, DC149 and DC807 were determined by cloning and by mass spectrometry. Alignment of the heavy and light chain sequences in DC2E7 and DC149 are shown in FIG. 27A-B. Alignments of the heavy and light chain sequences in DC2E7 and DC807 are shown in FIG. 28A-B. Residues identical to the sequence of DC2E7 are represented by dots. Complementarity-determining regions (CDRs) of VL and VH are boxed. Numbering of amino acid residues in VL and VH and the labeling of CDRs is according to the IMGT numbering system.

### LITERATURE REFERENCES

Hanes et al., (1998). Ribosome display efficiently selects and evolves high-affinity antibodies in vitro from immune libraries. PNAS, Vol. 95, pp. 14130-14135. doi.org/10.1073/pnas.95.24.14130.
Harrison et al., (1996). Screening of phage antibody libraries. vol. 26783-109, pp. 83-109. doi.org/10.1016/S0076-6879(96)67007-4.
Kabat, E. A., Wu, T. T., Perry, H. M., Gottesmann, K. S. & Foeller, C. (1991) in Sequences of Proteins of Immunological Interest (U.S. Department of Health and Human Services, Bethesda, Md.) Vol. I, pp. 151 and 464, 5th Ed.

### Example 19

### Independent Validation of pT217 Assay

The protocol for pT217 detection and quantitation discussed previously was applied to SIMOA using an HD-1 Analyzer. The protocol was found suitable for the measurement of pT217 in human CSF samples. The assay's sensitivity, linearity, parallelism, and recovery were analyzed. Standard curves were measured by spiking 2E7 pep calibrator peptide into CSF samples and into PBS. DC2E7 was used as the capture antibody and DC2E2 was used as the detection antibody. The observed limit of detection was 184.4 μg/mL. Repeatability was assessed: Intra-assay (<15% for values within the linear range of the standard curve), Inter-assay (<15% for values within the linear range of the standard curve), and Intra-plate (assay (<15% for values within the linear range of the standard curve). Parallelism and recovery were determined to be <15%.

1. An antibody or antigen binding fragment thereof capable of binding tau, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein
HCDR1 comprises the amino acid sequence of SEQ ID NO: 1, or SEQ ID NO: 1 with a substitution at one or more of position 5 and 6,
HCDR2 comprises the amino acid sequence of SEQ ID NO: 2, or SEQ ID NO: 2 with a substitution at one or more of position 1, 4, 5, 6, and 8,
HCDR3 comprises the amino acid sequence of SEQ ID NO: 3,
LCDR1 comprises the amino acid sequence of SEQ ID NO: 4, or SEQ ID NO: 4 with a substitution at position 2,
LCDR2 comprises the amino acid sequence of SEQ ID NO: 5, or SEQ ID NO: 5 with a substitution at position 3, and
LCDR3 comprises the amino acid sequence of SEQ ID NO: 6, or SEQ ID NO: 6 with a substitution at one or more of position 4 and 6.

2-4. (canceled)

5. The antibody or antigen binding fragment of claim 1, wherein
HCDR1 comprises the amino acid sequence of SEQ ID NO: 1, HCDR2 comprises the amino acid sequence of SEQ ID NO: 2, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 2, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 41; or
HCDR1 comprises the amino acid sequence of SEQ ID NO: 1, HCDR2 comprises the amino acid sequence of SEQ ID NO 34, HCDR3 comprises the amino acid sequence of SEQ ID NO: 3, LCDR1 comprises the amino acid sequence of SEQ ID NO: 4, LCDR2 comprise the amino acid sequence of SEQ ID NO: 5, and LCDR3 comprise the amino acid sequence of SEQ ID NO 6; or
HCDR1 comprises the amino acid sequence of SEQ ID NO: 32, HCDR2 comprises the amino acid sequence of SEQ ID NO: 2, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 35, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 2, HCDR3 comprises the amino acid sequence of SEQ ID NO: 3, LCDR1 comprises the amino acid sequence of SEQ ID NO: 4, LCDR2 comprises the amino acid sequence of SEQ ID NO: 40, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 36, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 33, HCDR2 comprises the amino acid sequence of SEQ ID NO: 37, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 2, HCDR3 comprises the amino acid sequence of SEQ ID NO: 3, LCDR1 comprises the amino acid sequence of SEQ ID NO: 39, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 38, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 42; or
HCDR1 comprises the amino acid sequence of SEQ ID NO: 33, HCDR2 comprises the amino acid sequence of SEQ ID NO: 37, HCDR3 comprises the amino acid sequence of SEQ ID NO: 3, LCDR1 comprises the amino acid sequence of SEQ ID NO: 4, LCDR2 comprises the amino acid sequence of SEQ ID NO: 5, and LCDR3 comprises the amino acid sequence of SEQ ID NO: 6; or
HCDR1 comprises the amino acid sequence of SEQ ID NO: 1, HCDR2 comprises the amino acid sequence of SEQ ID NO: 2, HCDR3 comprises the amino acid sequence of SEQ ID NO: 3, LCDR1 comprises the amino acid sequence of SEQ ID NO: 39, LCDR2 comprises the amino acid sequence of SEQ ID NO: 5, and LCDR3 comprises the amino acid sequence of SEQ ID NO: 6.

6-7. (canceled)

8. The antibody or antigen binding fragment of claim 1, wherein
the heavy chain variable region comprises the amino acid sequence of SEQ ID NO: 7 and the light chain variable region comprises the amino acid sequence of SEQ ID NO: 8; or
the heavy chain variable region comprises the amino acid sequence of SEQ ID NO: 43 and the light chain variable region comprises the amino acid sequence of SEQ ID NO: 44; or
the heavy chain variable region comprises the amino acid sequence of SEQ ID NO: 45 and the light chain variable region comprises the amino acid sequence of SEQ ID NO: 46; or
the heavy chain variable region comprises the amino acid sequence of SEQ ID NO: 47 and the light chain variable region comprises the amino acid sequence of SEQ ID NO: 48; or
the heavy chain variable region comprises the amino acid sequence of SEQ ID NO: 49 and the light chain variable region comprises the amino acid sequence of SEQ ID NO: 50; or
the heavy chain variable region comprises the amino acid sequence of SEQ ID NO: 51 and the light chain variable region comprises the amino acid sequence of SEQ ID NO: 52; or
the heavy chain variable region comprises the amino acid sequence of SEQ ID NO: 53 and the light chain variable region comprises the amino acid sequence of SEQ ID NO: 54; or
the heavy chain variable region comprises the amino acid sequence of SEQ ID NO: 55 and the light chain variable region comprises the amino acid sequence of SEQ ID NO: 56; or
the heavy chain variable region comprises the amino acid sequence of SEQ ID NO: 57 and the light chain variable region comprises the amino acid sequence of SEQ ID NO: 58; or
the heavy chain variable region comprises the amino acid sequence of SEQ ID NO: 59 and the light chain variable region comprises the amino acid sequence of SEQ ID NO: 60; or
the heavy chain variable region comprises the amino acid sequence of SEQ ID NO: 61 and the light chain variable region comprises the amino acid sequence of SEQ ID NO: 62; or
the heavy chain variable region comprises the amino acid sequence of SEQ ID NO: 63 and the light chain variable region comprises the amino acid sequence of SEQ ID NO: 64; or
the heavy chain variable region comprises the amino acid sequence of SEQ ID NO: 65 and the light chain variable region comprises the amino acid sequence of SEQ ID NO: 66; or
the heavy chain variable region comprises the amino acid sequence of SEQ ID NO: 67 and the light chain variable region comprises the amino acid sequence of SEQ ID NO: 68; or
the heavy chain variable region comprises the amino acid sequence of SEQ ID NO: 69 and the light chain variable region comprises the amino acid sequence of SEQ ID NO: 70; or
the heavy chain variable region comprises the amino acid sequence of SEQ ID NO: 71 and the light chain variable region comprises the amino acid sequence of SEQ ID NO: 72; or
the heavy chain variable region comprises the amino acid sequence of SEQ ID NO: 73 and the light chain variable region comprises the amino acid sequence of SEQ ID NO: 74; or
the heavy chain variable region comprises the amino acid sequence of SEQ ID NO: 75 and the light chain variable region comprises the amino acid sequence of SEQ ID NO: 76; or
the heavy chain variable region comprises the amino acid sequence of SEQ ID NO: 77 and the light chain variable region comprises the amino acid sequence of SEQ ID NO: 78; or
the heavy chain variable region comprises the amino acid sequence of SEQ ID NO: 79 and the light chain variable region comprises the amino acid sequence of SEQ ID NO: 80; or
the heavy chain variable region comprises the amino acid sequence of SEQ ID NO: 81 and the light chain variable region comprises the amino acid sequence of SEQ ID NO: 82; or
the heavy chain variable region comprises the amino acid sequence of SEQ ID NO: 83 and the light chain variable region comprises the amino acid sequence of SEQ ID NO: 84; or
the heavy chain variable region comprises the amino acid sequence of SEQ ID NO: 85 and the light chain variable region comprises the amino acid sequence of SEQ ID NO: 86; or
the heavy chain variable region comprises the amino acid sequence of SEQ ID NO: 87 and the light chain variable region comprises the amino acid sequence of SEQ ID NO: 88; or
the heavy chain variable region comprises the amino acid sequence of SEQ ID NO: 89 and the light chain variable region comprises the amino acid sequence of SEQ ID NO: 90; or
the heavy chain variable region comprises the amino acid sequence of SEQ ID NO: 91 and the light chain variable region comprises the amino acid sequence of SEQ ID NO: 92; or
the heavy chain variable region comprises the amino acid sequence of SEQ ID NO: 93 and the light chain variable region comprises the amino acid sequence of SEQ ID NO: 94; or
the heavy chain variable region comprises the amino acid sequence of SEQ ID NO: 95 and the light chain variable region comprises the amino acid sequence of SEQ ID NO: 96; or
the heavy chain variable region comprises the amino acid sequence of SEQ ID NO: 97 and the light chain variable region comprises the amino acid sequence of SEQ ID NO: 98.

9-10. (canceled)

11. An antibody or antigen binding fragment thereof capable of binding tau, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein:
HCDR1 comprises the amino acid sequence of SEQ ID NO: 15, HCDR2 comprises the amino acid sequence of SEQ ID NO: 16, HCDR3 comprises the amino acid sequence of SEQ ID NO: 17, LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20; or
HCDR1 comprises amino acid sequence of SEQ ID NO: 23, HCDR2 comprises the amino acid sequence of SEQ ID NO: 24, HCDR3 comprises the amino acid sequence of SEQ ID NO: 25, LCDR1 comprises the amino acid sequence of SEQ ID NO: 26, LCDR2 comprises the amino acid sequence of SEQ ID NO: 27, and LCDR3 comprises the amino acid sequence of SEQ ID NO: 28; or
HCDR1 comprises the amino acid sequence of SEQ ID NO: 101, HCDR2 comprises the amino acid sequence of SEQ ID NO: 102, HCDR3 comprises the amino acid sequence of SEQ ID NO: 103, LCDR1 comprises the amino acid sequence of SEQ ID NO: 104, LCDR2 comprises the amino acid sequence of SEQ ID NO: 105, and LCDR3 comprises the amino acid sequence of SEQ ID NO: 106.

12-29. (canceled)

30. The antibody or antigen binding fragment of claim 1, further comprising a heavy chain constant region and a light chain constant region, wherein the heavy chain constant region is a human IgG isotype heavy chain constant region and the light chain constant region is a human kappa light chain constant region.

31. (canceled)

32. The antibody or antigen binding fragment of claim 31 wherein the human IgG isotype is a human IgG1 isotype or a human IgG4 isotype.

33. (canceled)

34. The antibody or antigen binding fragment of claim 1, wherein the antibody or antigen binding fragment is selected from a rodent antibody or antigen binding fragment thereof, a chimeric antibody or an antigen binding fragment thereof, a CDR-grafted antibody or an antigen binding fragment thereof, and a humanized antibody or an antigen binding fragment thereof.

35. The antibody or antigen binding fragment of claim 1, wherein the antibody or antigen binding fragment is a Fab, Fab′, F(ab′)₂, Fd, scFv, (scFv)₂, scFv-Fc, or Fv fragment.

36. The antibody or antigen binding fragment of claim 1, wherein the antibody or antigen binding fragment is conjugated to a second agent.

37. The antibody or antigen binding fragment of claim 36, wherein the second agent is at least one detectable label, wherein the at least one detectable label comprises an enzyme, a radioisotope, a fluorophore, a biotin, a nuclear magnetic resonance marker, or a heavy metal.

38-40. (canceled)

41. An isolated nucleic acid encoding at least one variable region of an immunoglobulin chain of the antibody or antigen binding fragment of claim 1.

42. An isolated vector comprising the nucleic acid of claim 41.

43. An isolated host cell comprising the the vector of claim 42.

44. A method of producing an antibody or fragment thereof capable of binding tau, comprising culturing the host cell of claim 43 under conditions sufficient to produce the antibody or fragment thereof.

45. A pharmaceutical composition comprising one or more the antibody or antigen binding fragment of claim 1 and a pharmaceutically acceptable carrier and/or diluent.

46-47. (canceled)

48. A method of treating, delaying progression, or preventing the progression of Alzheimer's disease or another tauopathy in a subject, comprising administering to the subject an effective amount of the antibody or antigen binding fragment of claim 1.

49. (canceled)

50. A method of detecting a tauopathy in a subject comprising:
obtaining a biological sample from the subject;
contacting the biological sample with an effective amount of the antibody or antigen binding fragment of claim 1, and
detecting binding of the antibody or antigen binding fragment to tau in the biological sample, thereby detecting a tauopathy in the subject.

51-52. (canceled)

53. A method of detecting a tauopathy in a subject comprising:
obtaining a biological sample from the subject;
contacting the biological sample with an effective amount of the antibody or antigen binding fragment of claim 1 (“first antibody”), and
contacting the biological sample with a second antibody or antigen binding fragment capable of binding tau (“second antibody”),
detecting binding of the first and/or second antibody to tau in the biological sample,
thereby detecting a tauopathy in the subject.

54-65. (canceled)

66. The method of claim 53, wherein the heavy chain variable region of the first antibody comprises the amino acid sequence of SEQ ID NO: 7 and the light chain variable region of the first antibody comprises the amino acid sequence of SEQ ID NO: 8.

67-70. (canceled)

71. The method of claim 53, wherein the second antibody comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3), wherein HCDR1 comprises the amino acid sequence of SEQ ID NO: 15, HCDR2 comprises the amino acid sequence of SEQ ID NO: 16, HCDR3 comprises the amino acid sequence of SEQ ID NO: 17, LCDR1 comprises the amino acid sequence of SEQ ID NO: 18, LCDR2 comprises the amino acid sequence of SEQ ID NO: 19, and LCDR3 comprises the amino acid sequence of SEQ ID NO: 20.

72. The method of claim 71, wherein the heavy chain variable region of the second antibody comprises the amino acid sequence of SEQ ID NO: 21 and the light chain variable region of the second antibody comprises the amino acid sequence of SEQ ID NO: 22.

73-81. (canceled)

82. The method of claim 53, wherein the method comprises a classic ELISA, a digital ELISA, or a single molecule array.

83-88. (canceled)

89. The method of claim 53, wherein the method detects the amount of a phosphorylated tau in the biological sample, and the phosphorylated tau detected in the biological sample is (a) 1, 2, 3, 4, 5, 6, 7, 8, 9, 10, 15, 20, 25, or more fold higher than in a control sample, and/or (b) greater than a threshold of 0.1, 0.2, 0.3, 0.4, 0.5, 0.6, 0.7, 0.8, 0.9, 1, 1.5, 2, 2.5, 3, 4, 5, 6, 7, 8, 9, or 10 μg/ml.

90. The method of claim 53, wherein the method detects the amount of the phosphorylated tau in the biological sample to be greater than a threshold of about 100-600 μg/ml.

91. The method of claim 90, wherein the threshold is about 110, 120, 130, 140, 150, 160, 170, 180, 190, 200, 210, 220, 230, 240, 250, 260, 270, 280, 290, 300, 310, 320, 330, 340, 350, 360, 370, 380, 400, 410, 420, 430, 440, 450, 460, 470, 480, 490, 500, 510, 520, 530, 540, 550, 560, 570, 580, or 590 μg/ml.

92-97. (canceled)

98. A method of distinguishing Alzheimer's disease from another tauopathy or another cause of dementia in a subject, comprising:
obtaining a cerebrospinal fluid or blood sample from the subject;
performing the method of claim 53 to determine the amount of phosphorylated tau in the cerebrospinal fluid or blood sample; and
comparing the level of phosphorylated tau to the level in a control sample or to a threshold,
wherein an elevated level of phosphorylated tau in the cerebrospinal fluid or blood sample relative to the level in the control sample or threshold indicates the subject has Alzheimer's disease rather than another tauopathy or another cause of dementia.

99-106. (canceled)

107. A method of treatment, comprising administering a therapeutic agent for Alzheimer's disease to a subject suffering from Alzheimer's disease, wherein the subject has been identified as having Alzheimer's disease according to the method of claim 50.

108. A kit comprising the antibody or antigen binding fragment of claim 1 and instructions for using the antibody or antigen binding fragment to identify a subject having Alzheimer's disease or another tauopathy.

109. (canceled)

110. A method of detecting Alzheimer's disease or another tauopathy in a human subject, comprising administering to the subject the antibody or antigen binding fragment of claim 1 conjugated to a radioisotope and detecting a signal from the radioisotope in the brain of the subject, wherein detection of the signal indicates the subject has Alzheimer's disease or another tauopathy.

111-124. (canceled)

125. A method of determining the stage of Alzheimer's disease in a human subject, comprising:
obtaining a cerebrospinal fluid or blood sample from the subject;
performing the method of claim 53 to determine the amount of phosphorylated tau in the cerebrospinal fluid or blood sample; and
comparing the level of phosphorylated tau to the level in a cerebrospinal fluid or blood sample from a patient of known AD stage or a threshold level,
thereby identifying the stage of Alzheimer's disease.

126. (canceled)

127. A method of determining the effectiveness of an anti-tau therapy for Alzheimer's disease, comprising:
obtaining a cerebrospinal fluid or blood sample from a human subject;
performing the method of claim 53 to determine the amount of phosphorylated tau in the cerebrospinal fluid or blood sample; and
wherein an elevated level of phosphorylated tau in the cerebrospinal fluid or blood sample relative to the level in a cerebrospinal fluid or blood sample from a healthy control subject and/or relative to a threshold level indicates the subject is more likely to respond to an anti-tau therapy for Alzheimer's disease.

128-132. (canceled)

133. A method of monitoring the effectiveness of an anti-tau therapy for Alzheimer's disease, comprising:
a) obtaining a cerebrospinal fluid or blood sample from a human subject prior to treatment;
b) performing the method of claim 53 to determine the amount of phosphorylated tau in the cerebrospinal fluid or blood sample;
c) administering an anti-tau therapy to the subject;
d) repeating steps a)-b) after administering the anti-tau therapy, whereby a reduction in the level of phosphorylated tau in the cerebrospinal fluid or blood sample after treatment as compared to the level in the cerebrospinal fluid or blood sample before treatment indicates an effective therapy.

134-137. (canceled)

138. A hybridoma producing antibody DC2E7, wherein the hybridoma is deposited under American Type Culture Collection Patent Deposit No. PTA-124992.

139. A hybridoma producing antibody DC2E2, wherein the hybridoma is deposited under American Type Culture Collection Patent Deposit No. PTA-124991.

140. A method of detecting Alzheimer's disease (AD) or mild cognitive impairment (MCI) in a subject, comprising:
contacting a biological sample from the subject with an effective amount of the antibody or antigen binding fragment of claim 1 that is capable of binding tau to form a tau-antibody complex;
detecting the presence and/or amount of the tau-antibody complex; and
comparing the presence/amount of tau bound to the antibody in the biological sample to the amount in a control sample or a threshold,
wherein the presence and/or an increased amount of tau complexed with the antibody relative to the control sample or threshold indicates AD or MCI in the subject.

141. (canceled)

142. The method of claim 140, wherein the amount of the tau-antibody complex distinguishes AD and/or MCI from Parkinson's disease, Multiple sclerosis, Amyotrophic lateral sclerosis, and/or frontotemporal dementia in the subject.

143-167. (canceled)

168. A method of predicting the likelihood that a subject with mild cognitive impairment will develop Alzheimer's disease, comprising:
contacting a biological sample from the subject with an effective amount of the antibody or antigen binding fragment of claim 1 that is capable of binding tau to form a tau-antibody complex;
detecting the presence and/or amount of the tau-antibody complex; and
comparing the presence/amount of tau bound to the antibody in the biological sample to the amount in a control sample or a threshold,
wherein the presence and/or an increased amount of tau complexed with the antibody relative to the control sample or threshold indicates an increased likelihood that the subject will develop Alzheimer's disease.

169. A method of diagnosing or predicting Alzheimer's disease or a precursor thereof in a subject, comprising:
obtaining a biological sample from the subject;
detecting the presence and/or amount of tau protein 2N4R phosphorylated at least at position threonine 217 in the biological sample; and
comparing the presence/amount of tau protein 2N4R phosphorylated at threonine 217 in the biological sample to the amount in a control sample or a threshold,
wherein the presence and/or increased amount of tau protein 2N4R phosphorylated at least at threonine 217 in the biological sample relative to the control sample or threshold (a) indicates Alzheimer's disease or mild cognitive impairment in the subject and/or (b) wherein the subject has mild cognitive impairment, indicates an increased likelihood that the subject will develop Alzheimer's disease.

170-176. (canceled)
